SEQUENCE LISTING (184 sequences)

SEQ ID NO: 1 - protein, 25 aa, Artificial Sequence
  PR000184 HFWR1 Chothia
EVQLVESGGGLIQPGGSLRLSCAVS

SEQ ID NO: 2 - protein, 25 aa, Artificial Sequence [sequence omitted]

SEQ ID NO: 3 - protein, 25 aa, Artificial Sequence [sequence omitted]

SEQ ID NO: 4 - protein, 25 aa, Artificial Sequence
  PR000892 HFWR1 Chothia
QVQLVESGGGVVQPGRSLRLSCAAT

SEQ ID NO: 5 - protein, 25 aa, Artificial Sequence
  PR001760 HFWR1 Chothia
EVQLVESGGGVVQPGGSLRLSCAAS

SEQ ID NO: 6 - protein, 25 aa, Artificial Sequence
  PR002067 HFWR1 Chothia
EVQLLESGGGLVQPGGSLRLSCAAS

SEQ ID NO: 7 - protein, 25 aa, Artificial Sequence
  PR002408 HFWR1 Chothia
QVQLVESGGGVVQPGRSLRLSCAAS

SEQ ID NO: 8 - protein, 25 aa, Artificial Sequence
  PR003475 HFWR1 Chothia
EVQLVQSGAEVKKPGASVKVSCKAS

SEQ ID NO: 9 - protein, 25 aa, Artificial Sequence
  PR004433 HFWR1 Chothia
EVQLVETGGGLIQPGGSLRLSCAAS

SEQ ID NO: 10 - protein, 7 aa, Artificial Sequence
  PR000184 HCDR1 Chothia
GFTVSKN

SEQ ID NO: 11 - protein, 7 aa, Artificial Sequence
  PR000197 HCDR1 Chothia, PR000628 HCDR1 Chothia
GGSFSGY

SEQ ID NO: 12 - protein, 7 aa, Artificial Sequence
  PR000210 HCDR1 Chothia
GFNIKDT

SEQ ID NO: 13 - protein, 7 aa, Artificial Sequence
  PR000265 HCDR1 Chothia, PR000892 HCDR1 Chothia, PR002067 HCDR1 Chothia
GFTFSSY

SEQ ID NO: 14 - protein, 7 aa, Artificial Sequence
  PR001760 HCDR1 Chothia
GFTFSNY

SEQ ID NO: 15 - protein, 7 aa, Artificial Sequence
  PR002408 HCDR1 Chothia
GFTFRSF

SEQ ID NO: 16 - protein, 7 aa, Artificial Sequence
  PR003475 HCDR1 Chothia
GYTFTDS

SEQ ID NO: 17 - protein, 7 aa, Artificial Sequence
  PR004433 HCDR1 Chothia
GFTVSDS

SEQ ID NO: 18 - protein, 19 aa, Artificial Sequence
  PR000184 HFWR2 Chothia
YMSWVRQAPGKGLEWVSVV

SEQ ID NO: 19 - protein, 19 aa, Artificial Sequence
  PR000197 HFWR2 Chothia
YWSWIRQPPGKGLEWIGEI

SEQ ID NO: 20 - protein, 19 aa, Artificial Sequence
  PR000210 HFWR2 Chothia
YIHWVRQAPGKGLEWVARI

SEQ ID NO: 21 - protein, 19 aa, Artificial Sequence
  PR000265 HFWR2 Chothia
WMSWVRQAPGKGLEWVANI

SEQ ID NO: 22 - protein, 19 aa, Artificial Sequence
  PR000628 HFWR2 Chothia
YWSWIRQSPEKGLEWIGEI

SEQ ID NO: 23 - protein, 19 aa, Artificial Sequence
  PR000892 HFWR2 Chothia
GMYWVRQAPGKGLEWVAAI

SEQ ID NO: 24 - protein, 19 aa, Artificial Sequence
  PR001760 HFWR2 Chothia
AMTWVRQAPEKGLEWVSSI

SEQ ID NO: 25 - protein, 19 aa, Artificial Sequence
  PR002067 HFWR2 Chothia
AMSWVRQAPGKGLEWVSAI

SEQ ID NO: 26 - protein, 19 aa, Artificial Sequence
  PR002408 HFWR2 Chothia
GMHWVRQAPGKGLEWVAVI

SEQ ID NO: 27 - protein, 19 aa, Artificial Sequence
  PR003475 HFWR2 Chothia
YMSWVRQAPGQGLEWIGDM

SEQ ID NO: 28 - protein, 19 aa, Artificial Sequence
  PR004433 HFWR2 Chothia
YMTWVRQAPGKGLEWVSVI

SEQ ID NO: 29 - protein, 5 aa, Artificial Sequence
  PR000184 HCDR2 Chothia
YSGGS

SEQ ID NO: 30 - protein, 5 aa, Artificial Sequence
  PR000197 HCDR2 Chothia
NHSGS

SEQ ID NO: 31 - protein, 6 aa, Artificial Sequence
  PR000210 HCDR2 Chothia
YPTNGY

SEQ ID NO: 32 - protein, 6 aa, Artificial Sequence
  PR000265 HCDR2 Chothia
KQEGSE

SEQ ID NO: 33 - protein, 5 aa, Artificial Sequence
  PR000628 HCDR2 Chothia
NHGGY

SEQ ID NO: 34 - protein, 6 aa, Artificial Sequence
  PR000892 HCDR2 Chothia
WNDGSN

SEQ ID NO: 35 - protein, 6 aa, Artificial Sequence
  PR001760 HCDR2 Chothia
SGSGVS

SEQ ID NO: 36 - protein, 6 aa, Artificial Sequence
  PR002067 HCDR2 Chothia
SGRGGS

SEQ ID NO: 37 - protein, 6 aa, Artificial Sequence
  PR002408 HCDR2 Chothia
SYDASN

SEQ ID NO: 38 - protein, 6 aa, Artificial Sequence
  PR003475 HCDR2 Chothia
YPDNGD

SEQ ID NO: 39 - protein, 5 aa, Artificial Sequence
  PR004433 HCDR2 Chothia
FSGGR

SEQ ID NO: 40 - protein, 41 aa, Artificial Sequence
  PR000184 HFWR3 Chothia
KTYADSVKGRFTISRDNSKNTLYLQMNSLRAEDTAVYYCAR

SEQ ID NO: 41 - protein, 41 aa, Artificial Sequence
  PR000197 HFWR3 Chothia
TDSNPSLKGRVTFSVDTSKNQFSLKLNSVTAADTAVYYCAR

SEQ ID NO: 42 - protein, 41 aa, Artificial Sequence
  PR000210 HFWR3 Chothia
TRYADSVKGRFTISADTSKNTAYLQMNSLRAEDTAVYYCSR

SEQ ID NO: 43 - protein, 41 aa, Artificial Sequence
  PR000265 HFWR3 Chothia
KYYVDSVKGRFTISRDNAKNSLYLQMNSLRAEDTAVYYCAR

SEQ ID NO: 44 - protein, 41 aa, Artificial Sequence
  PR000628 HFWR3 Chothia
VTYNPSLESRVTISVDTSKNQFSLKLSSVTAADTAVYYCAR

SEQ ID NO: 45 - protein, 41 aa, Artificial Sequence
  PR000892 HFWR3 Chothia
NYYADSVKGRFTISRDDSKNTLNLQMNSLRAEDTAMYYCAR

SEQ ID NO: 46 - protein, 41 aa, Artificial Sequence
  PR001760 HFWR3 Chothia
TYYADSVKGRFTISRDNSKNTLYLQMTRLTAEDTAVYFCAK

SEQ ID NO: 47 - protein, 41 aa, Artificial Sequence
  PR002067 HFWR3 Chothia
TFYADSVRGRFTISRDNSKNTLYLQMNSLRAEDTAMYYCVD

SEQ ID NO: 48 - protein, 41 aa, Artificial Sequence
  PR002408 HFWR3 Chothia
EYYADSVKGRFIISRDNSKDTLYLQMNSLRAGDTAVYYCAK

SEQ ID NO: 49 - protein, 41 aa, Artificial Sequence
  PR003475 HFWR3 Chothia
SSYNQKFRERVTITRDTSTSTAYLELSSLRSEDTAVYYCVL

SEQ ID NO: 50 - protein, 41 aa, Artificial Sequence
  PR004433 HFWR3 Chothia
TYYSDSVKGRFTISRDNAKNTLYLQMNSLRAEDTALYYCAR

SEQ ID NO: 51 - protein, 11 aa, Artificial Sequence
  PR000184 HCDR3 Chothia
AVPHSPSSFDI

SEQ ID NO: 52 - protein, 7 aa, Artificial Sequence
  PR000197 HCDR3 Chothia
LTGPFDY

SEQ ID NO: 53 - protein, 11 aa, Artificial Sequence
  PR000210 HCDR3 Chothia
WGGDGFYAMDY

SEQ ID NO: 54 - protein, 10 aa, Artificial Sequence
  PR000265 HCDR3 Chothia
DRAVAGAFDI

SEQ ID NO: 55 - protein, 13 aa, Artificial Sequence
  PR000628 HCDR3 Chothia
DYGPGNYDWYFDL

SEQ ID NO: 56 - protein, 19 aa, Artificial Sequence
  PR000892 HCDR3 Chothia
DRLPMASLRYFDWLGVMDA

SEQ ID NO: 57 - protein, 15 aa, Artificial Sequence
  PR001760 HCDR3 Chothia
EGSSETDDHYYNVDV

SEQ ID NO: 58 - protein, 10 aa, Artificial Sequence
  PR002067 HCDR3 Chothia
GTTGTTDVDY

SEQ ID NO: 59 - protein, 10 aa, Artificial Sequence
  PR002408 HCDR3 Chothia
GGGLRWYFAY

SEQ ID NO: 60 - protein, 8 aa, Artificial Sequence
  PR003475 HCDR3 Chothia
APRWYFSV

SEQ ID NO: 61 - protein, 14 aa, Artificial Sequence
  PR004433 HCDR3 Chothia
RNYDDTRGTDVFDI

SEQ ID NO: 62 - protein, 11 aa, Artificial Sequence
  PR000184 HFWR4 Chothia, PR000265 HFWR4 Chothia, PR004433 HFWR4 Chothia
WGQGTMVTVSS

SEQ ID NO: 63 - protein, 11 aa, Artificial Sequence
  PR000197 HFWR4 Chothia, PR000210 HFWR4 Chothia, PR002067 HFWR4 Chothia, PR002408 HFWR4 Chothia, PR003475 HFWR4 Chothia
WGQGTLVTVSS

SEQ ID NO: 64 - protein, 11 aa, Artificial Sequence
  PR000628 HFWR4 Chothia
WGRGTLVTVSS

SEQ ID NO: 65 - protein, 11 aa, Artificial Sequence
  PR000892 HFWR4 Chothia
WGQGTSVTVSS

SEQ ID NO: 66 - protein, 11 aa, Artificial Sequence
  PR001760 HFWR4 Chothia
WGQGTTVTVSS

SEQ ID NO: 67 - protein, 23 aa, Artificial Sequence
  PR000197 LFWR1 Chothia
EFVMTQSPATLSVSPGERATLSC

SEQ ID NO: 68 - protein, 23 aa, Artificial Sequence
  PR000210 LFWR1 Chothia, PR003475 LFWR1 Chothia
DIQMTQSPSSLSASVGDRVTITC

SEQ ID NO: 69 - protein, 23 aa, Artificial Sequence
  PR000265 LFWR1 Chothia
DIQMTQSPSTLSASVGDRVTVTC

SEQ ID NO: 70 - protein, 23 aa, Artificial Sequence
  PR000628 LFWR1 Chothia
EIVLTQSPATLSLSPGERATLSC

SEQ ID NO: 71 - protein, 23 aa, Artificial Sequence
  PR000892 LFWR1 Chothia
DIQMTQSPSTLSASVGDRVTITC

SEQ ID NO: 72 - protein, 23 aa, Artificial Sequence
  PR002408 LFWR1 Chothia
EIVMTQSPATLSVSPGERATLSC

SEQ ID NO: 73 - protein, 11 aa, Artificial Sequence
  PR000197 LCDR1 Chothia
RASQSISSILA

SEQ ID NO: 74 - protein, 11 aa, Artificial Sequence
  PR000210 LCDR1 Chothia
RASQDVNTAVA

SEQ ID NO: 75 - protein, 11 aa, Artificial Sequence
  PR000265 LCDR1 Chothia
RASQSIYIWLA

SEQ ID NO: 76 - protein, 11 aa, Artificial Sequence
  PR000628 LCDR1 Chothia
RASQSVSSYLA

SEQ ID NO: 77 - protein, 11 aa, Artificial Sequence
  PR000892 LCDR1 Chothia
RASQSISSWLA

SEQ ID NO: 78 - protein, 11 aa, Artificial Sequence
  PR002408 LCDR1 Chothia
RASQSISSNLG

SEQ ID NO: 79 - protein, 11 aa, Artificial Sequence
  PR003475 LCDR1 Chothia
RASQDISNYLN

SEQ ID NO: 80 - protein, 15 aa, Artificial Sequence
  PR000197 LFWR2 Chothia, PR000628 LFWR2 Chothia, PR002408 LFWR2 Chothia
WYQQKPGQAPRLLIY

SEQ ID NO: 81 - protein, 15 aa, Artificial Sequence
  PR000210 LFWR2 Chothia, PR000892 LFWR2 Chothia, PR003475 LFWR2 Chothia
WYQQKPGKAPKLLIY

SEQ ID NO: 82 - protein, 15 aa, Artificial Sequence
  PR000265 LFWR2 Chothia
WYQQKPGKAPNLLIY

SEQ ID NO: 83 - protein, 7 aa, Artificial Sequence
  PR000197 LCDR2 Chothia, PR002408 LCDR2 Chothia
GASTRAT

SEQ ID NO: 84 - protein, 7 aa, Artificial Sequence
  PR000210 LCDR2 Chothia
SASFLYS

SEQ ID NO: 85 - protein, 7 aa, Artificial Sequence
  PR000265 LCDR2 Chothia
KASSLET

SEQ ID NO: 86 - protein, 7 aa, Artificial Sequence
  PR000628 LCDR2 Chothia
DASNRAT

SEQ ID NO: 87 - protein, 7 aa, Artificial Sequence
  PR000892 LCDR2 Chothia
KASSLES

SEQ ID NO: 88 - protein, 7 aa, Artificial Sequence
  PR003475 LCDR2 Chothia
YTSRLRS

SEQ ID NO: 89 - protein, 32 aa, Artificial Sequence
  PR000197 LFWR3 Chothia, PR002408 LFWR3 Chothia
GIPARFSGSGSGTEFTLTISSLQSEDFAVYYC

SEQ ID NO: 90 - protein, 32 aa, Artificial Sequence
  PR000210 LFWR3 Chothia
GVPSRFSGSRSGTDFTLTISSLQPEDFATYYC

SEQ ID NO: 91 - protein, 32 aa, Artificial Sequence
  PR000265 LFWR3 Chothia
GVPSRFSGSGSGTEFTLTISSLQPDDFATYYC

SEQ ID NO: 92 - protein, 32 aa, Artificial Sequence
  PR000628 LFWR3 Chothia
GIPARFSGSGSGTDFTLTISSLEPEDFAVYYC

SEQ ID NO: 93 - protein, 32 aa, Artificial Sequence
  PR000892 LFWR3 Chothia
GVPSRFSGSGSGTEFTLTISSLQTDDFATYYC

SEQ ID NO: 94 - protein, 32 aa, Artificial Sequence
  PR003475 LFWR3 Chothia
GVPSRFSGSGSGTDFTLTISSLQPEDFATYYC

SEQ ID NO: 95 - protein, 9 aa, Artificial Sequence
  PR000197 LCDR3 Chothia
QQYYNWPLT

SEQ ID NO: 96 - protein, 9 aa, Artificial Sequence
  PR000210 LCDR3 Chothia
QQHYTTPPT

SEQ ID NO: 97 - protein, 9 aa, Artificial Sequence
  PR000265 LCDR3 Chothia
QQYYGSSRT

SEQ ID NO: 98 - protein, 11 aa, Artificial Sequence
  PR000628 LCDR3 Chothia
QQRSNWPPALT

SEQ ID NO: 99 - protein, 9 aa, Artificial Sequence
  PR000892 LCDR3 Chothia
QQYNSYLFT

SEQ ID NO: 100 - protein, 10 aa, Artificial Sequence
  PR002408 LCDR3 Chothia
QQYQSWPPLT

SEQ ID NO: 101 - protein, 9 aa, Artificial Sequence
  PR003475 LCDR3 Chothia
QQGHTLPPT

SEQ ID NO: 102 - protein, 10 aa, Artificial Sequence
  PR000197 LFWR4 Chothia, PR000628 LFWR4 Chothia, PR002408 LFWR4 Chothia
FGGGTKVEIK

SEQ ID NO: 103 - protein, 10 aa, Artificial Sequence
  PR000210 LFWR4 Chothia, PR000265 LFWR4 Chothia, PR003475 LFWR4 Chothia
FGQGTKVEIK

SEQ ID NO: 104 - protein, 10 aa, Artificial Sequence
  PR000892 LFWR4 Chothia
FGQGTKLEIK

SEQ ID NO: 105 - protein, 119 aa, Artificial Sequence
  PR000184 VH
EVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSS

SEQ ID NO: 106 - protein, 115 aa, Artificial Sequence
  PR000197 VH
QVQLQQWGAGLLKPSETLSLTCAVYGGSFSGYYWSWIRQPPGKGLEWIGEINHSGSTDSNPSLKGRVTFSVDTSKNQFSLKLNSVTAADTAVYYCARLTGPFDYWGQGTLVTVSS

SEQ ID NO: 107 - protein, 120 aa, Artificial Sequence [sequence omitted]

SEQ ID NO: 108 - protein, 119 aa, Artificial Sequence
  PR000265 VH
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSS

SEQ ID NO: 109 - protein, 121 aa, Artificial Sequence [sequence omitted]

SEQ ID NO: 110 - protein, 128 aa, Artificial Sequence
  PR000892 VH
QVQLVESGGGVVQPGRSLRLSCAATGFTFSSYGMYWVRQAPGKGLEWVAAIWNDGSNNYYADSVKGRFTISRDDSKNTLNLQMNSLRAEDTAMYYCARDRLPMASLRYFDWLGVMDAWGQGTSVTVSS

SEQ ID NO: 111 - protein, 124 aa, Artificial Sequence
  PR001760 VH
EVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSS

SEQ ID NO: 112 - protein, 119 aa, Artificial Sequence
  PR002067 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 113 - protein, 119 aa, Artificial Sequence
  PR002408 VH
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSS

SEQ ID NO: 114 - protein, 117 aa, Artificial Sequence
  PR003475 VH
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 115 - protein, 122 aa, Artificial Sequence
  PR004433 VH
EVQLVETGGGLIQPGGSLRLSCAASGFTVSDSYMTWVRQAPGKGLEWVSVIFSGGRTYYSDSVKGRFTISRDNAKNTLYLQMNSLRAEDTALYYCARRNYDDTRGTDVFDIWGQGTMVTVSS

SEQ ID NO: 116 - protein, 107 aa, Artificial Sequence
  PR000197 VL
EFVMTQSPATLSVSPGERATLSCRASQSISSILAWYQQKPGQAPRLLIYGASTRATGIPARFSGSGSGTEFTLTISSLQSEDFAVYYCQQYYNWPLTFGGGTKVEIK

SEQ ID NO: 117 - protein, 107 aa, Artificial Sequence [sequence omitted]

SEQ ID NO: 118 - protein, 107 aa, Artificial Sequence
  PR000265 VL
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIK

SEQ ID NO: 119 - protein, 109 aa, Artificial Sequence [sequence omitted]

SEQ ID NO: 120 - protein, 107 aa, Artificial Sequence
  PR000892 VL
DIQMTQSPSTLSASVGDRVTITCRASQSISSWLAWYQQKPGKAPKLLIYKASSLESGVPSRFSGSGSGTEFTLTISSLQTDDFATYYCQQYNSYLFTFGQGTKLEIK

SEQ ID NO: 121 - protein, 108 aa, Artificial Sequence
  PR002408 VL
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIK

SEQ ID NO: 122 - protein, 107 aa, Artificial Sequence
  PR003475 VL
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 123 - protein, 351 aa, Artificial Sequence
  PR000184 HC
EVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 442 aa, Artificial Sequence
  PR000197 HC
QVQLQQWGAGLLKPSETLSLTCAVYGGSFSGYYWSWIRQPPGKGLEWIGEINHSGSTDSNPSLKGRVTFSVDTSKNQFSLKLNSVTAADTAVYYCARLTGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 125 - protein, 450 aa, Artificial Sequence
  PR000210 HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, Artificial Sequence
  PR000265 HC
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 448 aa, Artificial Sequence
  PR000628 HC
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 458 aa, Artificial Sequence
  PR000892 HC
QVQLVESGGGVVQPGRSLRLSCAATGFTFSSYGMYWVRQAPGKGLEWVAAIWNDGSNNYYADSVKGRFTISRDDSKNTLNLQMNSLRAEDTAMYYCARDRLPMASLRYFDWLGVMDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 356 aa, Artificial Sequence
  PR001760 HC
EVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 351 aa, Artificial Sequence
  PR002067 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, Artificial Sequence
  PR002408 HC
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 447 aa, Artificial Sequence
  PR003475 HC
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 354 aa, Artificial Sequence
  PR004433 HC
EVQLVETGGGLIQPGGSLRLSCAASGFTVSDSYMTWVRQAPGKGLEWVSVIFSGGRTYYSDSVKGRFTISRDNAKNTLYLQMNSLRAEDTALYYCARRNYDDTRGTDVFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 214 aa, Artificial Sequence
  PR000197 LC
EFVMTQSPATLSVSPGERATLSCRASQSISSILAWYQQKPGQAPRLLIYGASTRATGIPARFSGSGSGTEFTLTISSLQSEDFAVYYCQQYYNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 214 aa, Artificial Sequence
  PR000210 LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 214 aa, Artificial Sequence
  PR000265 LC, PR003550 Chain, PR004268 Chain, PR007163 Chain
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 216 aa, Artificial Sequence
  PR000628 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 214 aa, Artificial Sequence
  PR000892 LC
DIQMTQSPSTLSASVGDRVTITCRASQSISSWLAWYQQKPGKAPKLLIYKASSLESGVPSRFSGSGSGTEFTLTISSLQTDDFATYYCQQYNSYLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 215 aa, Artificial Sequence
  PR002408 LC, PR003335 Chain, PR004276 Chain, PR004278 Chain
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 214 aa, Artificial Sequence
  PR003475 LC
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 223 aa, Artificial Sequence
  PR000305 Chain, PR000653 Chain, PR000654 Chain, PR000655 Chain, PR000706 Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 142 - protein, 565 aa, Artificial Sequence
  PR000305 Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 572 aa, Artificial Sequence
  PR000653 Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGSEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 580 aa, Artificial Sequence
  PR000654 Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 580 aa, Artificial Sequence
  PR000655 Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTPPPPPEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 214 aa, Artificial Sequence
  PR000701 Chain
EIVMTQSPATLSVSPGERATLSCRASQSISSILAWYQQKPGQAPRLLIYGASTRATGIPARFSGSGSGTEFTLTISSLQSEDFAVYYCQQYYNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 222 aa, Artificial Sequence
  PR000701 Chain, PR004270 Chain, PR007164 Chain
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 148 - protein, 656 aa, Artificial Sequence
  PR000701 Chain
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQQWGAGLLKPSETLSLTCAVYGGSFSGYYWSWIRQPPGKGLEWIGEINHSGSTDSNPSLKGRVTFSVDTSKNQFSLKLNSVTAADTAVYYCARLTGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 572 aa, Artificial Sequence
  PR000706 Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGSEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 589 aa, Artificial Sequence
  PR003335 Chain
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSS

SEQ ID NO: 151 - protein, 589 aa, Artificial Sequence
  PR003550 Chain
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSS

SEQ ID NO: 152 - protein, 588 aa, Artificial Sequence
  PR004268 Chain
EVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSGGGGSGGGGSGGGGSEVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 585 aa, Artificial Sequence
  PR004270 Chain
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTPPPPPEVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 584 aa, Artificial Sequence
  PR004276 Chain
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 155 - protein, 222 aa, Artificial Sequence
  PR004277 Chain, PR004279 Chain
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 156 - protein, 581 aa, Artificial Sequence
  PR004277 Chain
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTPPPPPEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 588 aa, Artificial Sequence
  PR004278 Chain
EVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSGGGGSGGGGSGGGGSQVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 586 aa, Artificial Sequence
  PR004279 Chain
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTPPPPPEVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 231 aa, Artificial Sequence
  PR005744 Chain
QVQLVESGGGVVQPGRSLRLSCAATGFTFSSYGMYWVRQAPGKGLEWVAAIWNDGSNNYYADSVKGRFTISRDDSKNTLNLQMNSLRAEDTAMYYCARDRLPMASLRYFDWLGVMDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 160 - protein, 568 aa, Artificial Sequence
  PR005744 Chain
DIQMTQSPSTLSASVGDRVTITCRASQSISSWLAWYQQKPGKAPKLLIYKASSLESGVPSRFSGSGSGTEFTLTISSLQTDDFATYYCQQYNSYLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVETGGGLIQPGGSLRLSCAASGFTVSDSYMTWVRQAPGKGLEWVSVIFSGGRTYYSDSVKGRFTISRDNAKNTLYLQMNSLRAEDTALYYCARRNYDDTRGTDVFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 4 aa, Artificial Sequence
  GS_4
GSGS

SEQ ID NO: 162 - protein, 5 aa, Artificial Sequence
  GS_5
GGGGS

SEQ ID NO: 163 - protein, 7 aa, Artificial Sequence
  GS_7
GGGGSGS

SEQ ID NO: 164 - protein, 15 aa, Artificial Sequence
  GS_15
GGGGSGGGGSGGGGS

SEQ ID NO: 165 - protein, 20 aa, Artificial Sequence
  GS_20
GGGGSGGGGSGGGGSGGGGS

SEQ ID NO: 166 - protein, 25 aa, Artificial Sequence
  GS_25
GGGGSGGGGSGGGGSGGGGSGGGGS

SEQ ID NO: 167 - protein, 15 aa, Artificial Sequence
  H1_15
EPKSSDKTHTPPPPP

SEQ ID NO: 168 - protein, 10 aa, Artificial Sequence
  LH1
DKTHTCPPCP

SEQ ID NO: 169 - protein, 15 aa, Artificial Sequence
  G5-LH
GGGGGDKTHTCPPCP

SEQ ID NO: 170 - protein, 17 aa, Artificial Sequence
  H1_15-RT
EPKSSDKTHTPPPPPRT

SEQ ID NO: 171 - protein, 18 aa, Artificial Sequence
  L-GS_15-RT
LGGGGSGGGGSGGGGSRT

SEQ ID NO: 172 - protein, 18 aa, Artificial Sequence
  L-H1_15-RT
LEPKSSDKTHTPPPPPRT

SEQ ID NO: 173 - protein, 19 aa, Artificial Sequence
  KL-H1_15-RT
KLEPKSSDKTHTPPPPPRT

SEQ ID NO: 174 - protein, 19 aa, Artificial Sequence
  KL-H1_15-AS
KLEPKSSDKTHTPPPPPAS

SEQ ID NO: 175 - protein, 9 aa, Artificial Sequence
  RT-GS_5-KL
RTGGGGSKL

SEQ ID NO: 176 - protein, 19 aa, Artificial Sequence
  RT-GS_15-KL
RTGGGGSGGGGSGGGGSKL

SEQ ID NO: 177 - protein, 29 aa, Artificial Sequence
  RT-GS_25-KL
RTGGGGSGGGGSGGGGSGGGGSGGGGSKL

SEQ ID NO: 178 - protein, 15 aa, Artificial Sequence
  Human IgG1 hinge
EPKSCDKTHTCPPCP

SEQ ID NO: 179 - protein, 15 aa, Artificial Sequence
  Human IgG1 hinge (C220S)
EPKSSDKTHTCPPCP

SEQ ID NO: 180 - protein, 12 aa, Artificial Sequence
  Human IgG2 hinge
ERKCCVECPPCP

SEQ ID NO: 181 - protein, 12 aa, Artificial Sequence
  Human IgG4 hinge
ESKYGPPCPSCP

SEQ ID NO: 182 - protein, 12 aa, Artificial Sequence
  Human IgG4 hinge (S228P)
ESKYGPPCPPCP

SEQ ID NO: 183 - protein, 578 aa, Artificial Sequence
  PR007163 Chain
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 570 aa, Artificial Sequence
  PR007164 Chain
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGVVQPGGSLRLSCAASGFTFSNYAMTWVRQAPEKGLEWVSSISGSGVSTYYADSVKGRFTISRDNSKNTLYLQMTRLTAEDTAVYFCAKEGSSETDDHYYNVDVWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0287143 A1
(43) Pub. Date: Sep. 14, 2023

(54) BINDING PROTEIN IN FAB-HCAB STRUCTURE

(71) Applicant: HARBOUR BIOMED (SHANGHAI) CO., LTD, Shanghai (CN)

(72) Inventors: Yun HE, Shanghai (CN); Lei SHI, Shanghai (CN); Bing HUANG, Shanghai (CN); Chen ZHONG, Shanghai (CN); Qiang LV, Shanghai (CN); Ming WANG, Shanghai (CN)

(21) Appl. No.: 18/013,530

(22) PCT Filed: Jun. 29, 2021
(86) PCT No.: PCT/CN2021/102935

§ 371 Date: Dec. 28, 2022

(30) Foreign Application Priority Data
Jun. 30, 2020 (CN) 202010618158.0
Jun. 30, 2020 (CN) 202010630471.6
Dec. 8, 2020 (CN) 202011423832.6

(51) Int. Cl. C07K 16/46; C07K 16/28; C07K 16/30; C12N 15/70; C12N 15/85

(52) U.S. Cl. CPC C07K 16/46; C07K 16/2827; C07K 16/3015; C12N 15/70; C12N 15/85; C07K 2317/31; C07K 2317/55; C07K 2317/565

(57) ABSTRACT

Provided is a binding protein, which comprises a protein functional region A and a protein functional region B, both of which target different antigens or different epitopes of antigens. The protein functional region A is Fab; the protein functional region B is VH; and the binding protein also comprises an Fc homodimer. There are two of both the protein functional region A and the protein functional region B. The binding protein has a left-right symmetrical structure. The binding protein consists of the protein functional region A, the protein functional region B and Fc in sequence from the N-terminus to the C-terminus, wherein the protein functional region A and the protein functional region B are connected by means of L1, and the protein functional region B is connected to the Fc by means of L2. The binding protein has a relatively small molecular mass, few polypeptide chains and a simpler structure. The protein has a versatile structure and may be applied to a variety of different target combinations. Compared with bispecific binding proteins in other structures, the binding protein has stronger ability to activate effector cells.

The present application claims priority to Chinese Patent Application No. 202010618158.0 filed on Jun. 30, 2020, priority to Chinese Patent Application No. 202010630471.6 filed on Jun. 30, 2020, and priority to Chinese Patent Application No. 202011423832.6 filed on Dec. 8, 2020, the content of which is incorporated herein by reference in its entirety.

### TECHNICAL FIELD

The present invention relates to the field of biomedicine, in particular to a binding protein with a Fab-HCAb structure, preparation therefor and use thereof.

### BACKGROUND

Antibodies are immunoglobulins (Igs) produced by B cells of the immune system upon stimulation by antigens, which can specifically bind to the corresponding antigens. The basic structure of antibodies of most species is in a “Y”-type tetrameric form, comprising two identical heavy chains (H chains) and two identical light chains (L chains), which are also referred to as “H2L2”. The heavy chain comprises a heavy chain variable region (VH) near the N-terminus and a heavy chain constant region (CH) near the C-terminus; and the light chain comprises a light chain variable region (VL) near the N-terminus and a light chain constant region (CL) near the C-terminus. The heavy chain constant region of IgG antibody has 3 domains, namely CH1, CH2 and CH3; and has a hinge region between CH1 and CH2. The variable region of the antibody is the primary site where it recognizes and binds to the antigen; and the domains VH and VL of variable regions and the domains CH1 and CL of constant regions of the antibody together constitute the antigen-binding fragment (Fab). The CH2 and CH3 constitute the fragment crystallizable (Fc), which is the primary site where the effector functions of antibody are exerted and the serum half-life of antibody is affected.

A heavy-chain antibody (HCAb) lacking light chains is naturally present in the serum of Camelidae species and sharks. The heavy-chain antibody derived from Camelidae species has no CH1 region between the heavy chain variable region and the hinge region thereof, and contains only one heavy chain variable region (VHH) and two heavy chain constant domains (CH2 and CH3), in addition to the lack of light chains, as compared to the conventional antibodies; and the basic structure thereof is a heavy chain dimer. The VHH fragment of the heavy-chain antibody of Camelidae species has different characteristics from the VH of the conventional antibodies, and the VHH structure cloned and expressed separately has structural stability and antigen-binding activity that are comparable to the original heavy-chain antibody. The heavy-chain antibody of Camelidae species has a molecular weight of only about 13 KDa, and is therefore also referred to as a nanobody or a single-domain antibody. The heavy-chain antibody or the nanobody derived therefrom have unique advantages in molecular imaging, diagnostic reagents, etc., but its therapeutic uses are limited by its non-human nature and potential immunogenic risk, which requires further antibody engineering (e.g., antibody humanization) to meet the requirements for clinical treatment.

Since the human antibody has a natural structure of “H2L2”, the association of VH and VL ensures the stability and solubility of the antibody. If there is no VL, the hydrophobic groups on the VH that would otherwise be protected by VL will be exposed to aqueous solvents, which makes VH prone to aggregation, thereby leading to poor antibody solubility. Therefore, it is impossible to obtain functional human heavy-chain antibodies from natural sources. Frank Grosveld et al. proposed a method for obtaining a fully human heavy-chain antibody using a transgenic animal (Patent Application WO2007/096779). Frank Grosveld et al. constructed a transgenic mouse, specifically, the endogenous antibody heavy chain locus and light chain locus of the mouse were both knocked out or inactivated, making it impossible to produce mouse antibodies; then, the heavy chain gene fragments of human antibody (V, D, and J fragments) were transferred into the mouse to produce an antibody with a human antibody gene sequence by the rearrangement and mutation mechanisms of the mouse itself, and the produced antibody was the human heavy-chain antibody due to the absence of the light chain. The VDJ combinations and mutations which are beneficial to the solubility of VH can be selected by introducing gene mutations and performing natural selection after VDJ rearrangement in the transgenic mouse, so as to effectively improve the solubility of VH. Therefore, a non-naturally occurring human heavy-chain dimer structure can be produced in the transgenic mouse. The fully human heavy-chain antibody obtained from the transgenic mouse and the fully human single-domain antibody derived therefrom have wide application prospect.

Bispecific antibodies and multispecific antibodies are a class of artificial antibodies with two or more different specific antigen-binding sites prepared by protein engineering techniques based on natural monoclonal antibodies. The natural monoclonal antibodies are monospecific, i.e., capable of recognizing and binding to only one antigen; the bispecific antibodies can bind to two different antigens or different epitopes on the same antigen; while the multispecific antibodies may recognize more antigens. This allows bispecific antibodies to achieve mechanisms of action and functional effects that cannot be achieved by some monospecific antibodies, thereby greatly expanding the therapeutic application scenarios for bispecific antibodies. With the rise of tumor immunology in recent years, bispecific antibodies have attracted increasing attention and technical and financial support, becoming the fastest growing field in the therapeutic antibody market.

The structural design of the bispecific antibodies is very important. Naturally occurring bivalent IgG antibodies consist of two identical heavy chains and two identical light chains, and contain two identical antigen-binding sites. Bispecific antibodies require the introduction of two different antigen-binding sites by structural design using means such as protein engineering techniques, resulting in molecules whose polypeptide chains are derived from two different heavy chains and two different light chains. Therefore, the most major challenge in the development of bispecific antibodies is the problem of mismatching of chains, that is, how to obtain a functional bispecific antibody with the correct chain combination from more than 10 different combinations of heavy and light chains. In order to solve this problem, scientists have developed a variety of development strategies and technical platforms to improve the homogeneity and yield of desired target products by introducing different design features or functional characteristics. The adoption of a symmetric structure is a strategy to solve the problem of mismatching of chains. Most of the symmetric structures are designed with a “2+2” structure, also referred to as a “tetravalent bispecific” symmetric structure. Those molecules with symmetric structures differ greatly in molecular size and pharmaceutical properties due to the possible different structures, orientations and positions of their antigen-binding domains. The symmetric structure still has the problem of light chain mismatch. Therefore, the DVD-Ig technical platform from AbbVie, the FIT-Ig technical platform from EpimAb, the WuXiBody technical platform from WuXi Biologics and the like solve the problem of light chain mismatch by adopting different strategies; and companies such as Aptevo and MedImmune solve the problem of light chain mismatch by introducing an scFv structure. However, all technical approaches have their limitations. For example, the bispecific antibody molecules produced by FIT-Ig and other techniques are at a relatively large molecular weight of about 250 KDa, which may influence the endocytosis, tissue penetration and other capacities of the bispecific antibody molecules; the introduction of scFv structure may have an impact on stability and solubility; furthermore, many technical platforms produce bispecific antibodies with at least three different polypeptide chains, which increases the complexity of the molecules.

Therefore, there is still an urgent need to develop a novel bispecific antibody molecular structure having a simpler and more stable molecular structure and excellent pharmaceutical properties to meet the requirements of rapid development and low production cost.

Heavy-chain antibodies and single-domain antibodies derived therefrom have unique advantages in the construction of bispecific or even multispecific antibodies. The heavy-chain antibody has an antigen-binding domain that is only one quarter the size of the Fab of a conventional antibody, and does not have light chain, so that the problem of light chain mismatch is avoided. Therefore, bispecific or even multispecific antibodies with smaller molecular weight, less polypeptide chains and simpler structures can be constructed using heavy-chain antibodies and single-domain antibodies derived therefrom. Furthermore, fully human heavy-chain antibodies are more advantageous in terms of immunogenicity and druggability than heavy-chain antibodies of Camelidae species.

### SUMMARY

In order to overcome the defect of the lack of the bispecific binding proteins with simple structure and stable and excellent pharmaceutical properties in the prior art, the present invention provides a bispecific binding protein with a “Fab-HCAb structure”, a preparation method therefor and use thereof. The “Fab-HCAb structure” has the characteristics of a relatively small molecular weight, less polypeptide chains, simple structure and the like, and also has the similar Fc effector function to the IgG antibody, excellent molecular stability and pharmaceutical properties, and the like.

In order to solve the above technical problems, a first technical solution of the invention is as follows: provided is a binding protein comprising at least two protein functional regions, wherein the binding protein comprises a protein functional region A and a protein functional region B; the protein functional region A and the protein functional region B target different antigens or different epitopes on the same antigen, the protein functional region A is of a Fab structure, and the protein functional region B is of a VH structure; the binding protein further comprises an Fc homodimer (comprising at least one Fc);
wherein the number of the protein functional region A is two, and the number of the protein functional region B is two; the binding protein is of a symmetrical structure, and the symmetrical structure is a left-right symmetric structure;
the binding protein comprises a protein functional region A, a protein functional region B and Fc sequentially from the N-terminus to the C-terminus, wherein the protein functional region A is linked to the protein functional region B via a first linker peptide (L1), and the protein functional region B is linked to the Fc via a second linker peptide (L2).

In the binding protein of the present invention, the two protein functional regions B form a symmetric dimeric form of a single chain antibody with the Fc, and the protein functional region A is linked to the N-terminus of the dimer of the single chain antibody, in which case the protein functional region A may be linked to the N-terminus of the protein functional region B with its CH1 (see, for example, FIG. 1, structure (2)) or CL (see, for example, FIG. 1, structure (1)).

In the present invention, the binding protein may be a tetravalent binding protein, for example, with a structure as shown in FIG. 1, structure (1) or structure (2); the binding protein has two different polypeptide chains.

Preferably, the binding protein has four polypeptide chains, including two identical short chains (or referred to as “polypeptide chains 1”) and two identical long chains (or referred to as “polypeptide chains 2”), wherein (1) the short chain (or referred to as “polypeptide chain 1”) comprises VH_A-CH1 sequentially from the N-terminus to the C-terminus, and the long chain (or referred to as “polypeptide chain 2”) comprises VL_A-CL-L1-VH_B-L2-CH2-CH3 sequentially from the N-terminus to the C-terminus; or (2) the short chain (or referred to as “polypeptide chain 1”) comprises VL_A-CL sequentially from the N-terminus to the C-terminus, and the long chain (or referred to as “polypeptide chain 2”) comprises VH_A-CH1-L1-VH_B-L2-CH2-CH3 sequentially from the N-terminus to the C-terminus. In the structure (1), the protein functional region A is linked to the N-terminus of the protein functional region B with the C-terminus of its CL, and VL_A of the protein functional region A and VH_B of the protein functional region B are fused on the same polypeptide chain, so that the mismatched byproducts from the association of VL_A and VH_B is more possibly avoided in this structure than in the structure (2).

The VL, VH, CL and CH herein all have conventional meanings in the art and represent light chain variable region, heavy chain variable region, light chain constant region and heavy chain constant region, respectively, wherein the CH includes CH1, CH2 and CH3, which are the first, second and third domains of the heavy chain constant region, respectively; the CL is a light chain constant region domain; _A and _B represent the functional regions as a protein functional region A and a protein functional region B or compositions thereof, respectively (that is, VH_A represents the heavy chain variable region of the protein functional region A, VH_B represents the heavy chain variable region of the protein functional region B, and VL_A represents the light chain variable region of the protein functional region A); “-” represents a polypeptide bond linking different structural regions or is used to separate different structural regions; the C-terminus is the carboxyl-terminus of the peptide chain (which may also be written as “C”), and the N-terminus is the amino-terminus of the peptide chain (which may also be written as “N′”). The different protein functional regions are fused on the same polypeptide chain, so that mismatched byproducts can be avoided. In some embodiments, L1 and L2 may be identical sequences. In other embodiments, L1 and L2 may be different sequences. When the L1 and/or L2 is “-”, the linker peptide has a length of 0. Preferably, the L1 (first linker peptide) and L2 (second linker peptide) may independently be, for example, “-” or GS or have amino acid sequences as shown in SEQ ID NOs: 161-182, respectively. In some embodiments, the L1 may preferably be 0 in length or have an amino acid sequence as set forth in SEQ ID NO: 163, 164 or 167. In some embodiments, the L2 may preferably have an amino acid sequence as set forth in SEQ ID NO: 169, 178 or 179. In some embodiments, the L1 and L2 have amino acid sequences as set forth in SEQ ID NO: 167 and SEQ ID NO: 179, respectively. In some embodiments, the L1 is 0 in length and the L2 has an amino acid sequence as set forth in SEQ ID NO: 178. In some embodiments, the L1 is 0 in length and the L2 has an amino acid sequence as set forth in SEQ ID NO: 179. In some embodiments, the L1 and L2 have amino acid sequences as set forth in SEQ ID NO: 163 and SEQ ID NO: 178, respectively. In some embodiments, the L1 and L2 have amino acid sequences as set forth in SEQ ID NO: 164 and SEQ ID NO: 178, respectively. In some embodiments, the L1 and L2 have amino acid sequences as set forth in SEQ ID NO: 167 and SEQ ID NO: 178, respectively. In some embodiments, the L1 and L2 have amino acid sequences as set forth in SEQ ID NO: 163 and SEQ ID NO: 169, respectively.

In some specific embodiments, the protein functional region A is also referred to as an antibody A against a first antigen or a first antigen-binding domain; the protein functional region B is also referred to as an antibody B against a second antigen or a second antigen-binding domain.

In some specific embodiments, the bispecific binding protein of the “Fab-HCAb structure” comprises at least one heavy chain variable region domain VH derived from a human heavy-chain antibody, and is capable of binding to two or more antigens, or two or more epitopes on the same antigen, or two or more copies on the same epitope.

In some specific embodiments, the bispecific binding protein of the “Fab-HCAb structure” comprises a heavy chain constant region which is preferably a human IgG1, human IgG2, human IgG3 or human IgG4 heavy chain constant region or a mutation thereof, wherein the mutation is preferably one or more mutations selected from C220S, N297A, L234A, L235A, G237A and P329G, and sites of the mutations are numbered according to the EU numbering scheme. For example, the heavy chain constant region may comprise one, two or three mutations of L234A, L235A, G237A, N297A and P329G, e.g., a combination of mutations comprising L234A and L235A (LALA), a combination of mutations comprising L234A, L235A and P329G (AAG), a combination of mutations comprising L234A, L235A and G237A (AAA), or the like.

In some specific embodiments, the antigen is selected from one or more of PD-L1, HER2, B7H4, CTLA4, OX40, 4-1BB and BCMA. The binding protein comprises at least two protein functional regions, namely a protein functional region A and a protein functional region B, wherein the protein functional region A and the protein functional region B are independently derived from one or more of a PD-L1 antibody or an antigen-binding fragment thereof, an HER2 antibody or an antigen-binding fragment thereof, a B7H4 antibody or an antigen-binding fragment thereof, a CTLA4 antibody or an antigen-binding fragment thereof, an OX40 antibody or an antigen-binding fragment thereof, a 4-1BB antibody or an antigen-binding fragment thereof, and a BCMA antibody or an antigen-binding fragment thereof. Preferably, the protein functional region A is Fab derived from a PD-L1 antibody or an antigen-binding fragment thereof, an HER2 antibody or an antigen-binding fragment thereof, a B7H4 antibody or an antigen-binding fragment thereof, or a BCMA antibody or an antigen-binding fragment thereof, and/or the protein functional region B is VH derived from a CTLA4 antibody or an antigen-binding fragment thereof, a 4-1BB antibody or an antigen-binding fragment thereof, an OX40 antibody or an antigen-binding fragment thereof, or a BCMA antibody or an antigen-binding fragment thereof. More preferably, in the binding protein: the protein functional region A is Fab derived from an HER2 antibody or an antigen-binding fragment thereof, and the protein functional region B is VH derived from a CTLA4 antibody or an antigen-binding fragment thereof; or, the protein functional region A is Fab derived from a PD-L1 antibody or an antigen-binding fragment thereof, and the protein functional region B is VH derived from a 4-1BB antibody or an antigen-binding fragment thereof; or, the protein functional region A is Fab derived from a B7H4 antibody or an antigen-binding fragment thereof, and the protein functional region B is VH derived from a 4-1BB antibody or an antigen-binding fragment thereof; or, the protein functional region A is Fab derived from a B7H4 antibody or an antigen-binding fragment thereof, and the protein functional region B is VH derived from an OX40 antibody or an antigen-binding fragment thereof; or, the protein functional region A is Fab derived from a BCMA antibody or an antigen-binding fragment thereof, and the protein functional region B is VH derived from a BCMA antibody or an antigen-binding fragment thereof.

In some specific embodiments, the PD-L1 antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 75, 85 and 97, respectively; and the VH comprises HCDR1, HCDR2 and HCDR3 with amino acid sequences as set forth in SEQ ID NOs: 13, 32 and 54, respectively. The amino acid sequences of the listed CDRs are shown according to the Chothia scheme.

In some specific embodiments, the B7H4 antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 78, 83 and 100, respectively; and the VH comprises HCDR1, HCDR2 and HCDR3 with amino acid sequences as set forth in SEQ ID NOs: 15, 37 and 59, respectively. The amino acid sequences of the listed CDRs are shown according to the Chothia scheme.

In some specific embodiments, the 4-1BB antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 73, 83 and 95, respectively; and the VH comprises HCDR1, HCDR2 and HCDR3 with amino acid sequences as set forth in SEQ ID NOs: 11, 30 and 52, respectively. The amino acid sequences of the listed CDRs are shown according to the Chothia scheme.

In some specific embodiments, the 4-1BB antibody or the antigen-binding fragment thereof comprises a heavy chain variable region (VH), wherein the VH comprises HCDR1, HCDR2 and HCDR3 with amino acid sequences as set forth in SEQ ID NOs: 14, 35 and 57, respectively. The amino acid sequences of the listed CDRs are shown according to the Chothia scheme.

In some specific embodiments, the OX40 antibody or the antigen-binding fragment thereof comprises a heavy chain variable region (VH), wherein the VH comprises HCDR1, HCDR2 and HCDR3 with amino acid sequences as set forth in SEQ ID NOs: 13, 36 and 58, respectively. The amino acid sequences of the listed CDRs are shown according to the Chothia scheme.

In some specific embodiments, the BCMA antibody or the antigen-binding fragment thereof comprises a heavy chain variable region (VH), wherein the VH comprises HCDR1, HCDR2 and HCDR3 with amino acid sequences as set forth in SEQ ID NOs: 17, 39 and 61, respectively. The amino acid sequences of the listed CDRs are shown according to the Chothia scheme.

In some specific embodiments, the BCMA antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 77, 87 and 99, respectively; and the VH comprises HCDR1, HCDR2 and HCDR3 with amino acid sequences as set forth in SEQ ID NOs: 13, 34 and 56, respectively. The amino acid sequences of the listed CDRs are shown according to the Chothia scheme.

In some specific embodiments, the CTLA4 antibody or the antigen-binding fragment thereof comprises a heavy chain variable region (VH), wherein the VH comprises HCDR1, HCDR2 and HCDR3 with amino acid sequences as set forth in SEQ ID NOs: 10, 29 and 51, respectively. The amino acid sequences of the listed CDRs are shown according to the Chothia scheme.

In some specific embodiments, the HER2 antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 74, 84 and 96, respectively; and the VH comprises HCDR1, HCDR2 and HCDR3 with amino acid sequences as set forth in SEQ ID NOs: 12, 31 and 53, respectively. The amino acid sequences of the listed CDRs are shown according to the Chothia scheme.

In some specific embodiments, the PD-L1 antibody or the antigen-binding fragment thereof comprises a light chain variable region (VL) and a heavy chain variable region (VH), wherein the VL comprises an amino acid sequence as set forth in SEQ ID NO: 118, and the VH comprises an amino acid sequence as set forth in SEQ ID NO: 108.

In some specific embodiments, the B7H4 antibody or the antigen-binding fragment thereof comprises a light chain variable region (VL) and a heavy chain variable region (VH), wherein the VL comprises an amino acid sequence as set forth in SEQ ID NO: 121, and the VH comprises an amino acid sequence as set forth in SEQ ID NO: 113.

In some specific embodiments, the 4-1BB antibody or the antigen-binding fragment thereof comprises a light chain variable region (VL) and a heavy chain variable region (VH), wherein the VL comprises an amino acid sequence as set forth in SEQ ID NO: 116, and the VH comprises an amino acid sequence as set forth in SEQ ID NO: 106.

In some specific embodiments, the 4-1BB antibody or the antigen-binding fragment thereof comprises a heavy chain variable region (VH), wherein the VH comprises an amino acid sequence as set forth in SEQ ID NO: 111.

In some specific embodiments, the OX40 antibody or the antigen-binding fragment thereof comprises a heavy chain variable region (VH), wherein the VH comprises an amino acid sequence as set forth in SEQ ID NO: 112.

In some specific embodiments, the BCMA antibody or the antigen-binding fragment thereof comprises a heavy chain variable region (VH), wherein the VH comprises an amino acid sequence as set forth in SEQ ID NO: 115.

In some specific embodiments, the BCMA antibody or the antigen-binding fragment thereof comprises a light chain variable region (VL) and a heavy chain variable region (VH), wherein the VL comprises an amino acid sequence as set forth in SEQ ID NO: 120, and the VH comprises an amino acid sequence as set forth in SEQ ID NO: 110.

In some specific embodiments, the CTLA4 antibody or the antigen-binding fragment thereof comprises a heavy chain variable region (VH), wherein the VH comprises an amino acid sequence as set forth in SEQ ID NO: 105.

In some specific embodiments, the HER2 antibody or the antigen-binding fragment thereof comprises a light chain variable region (VL) and a heavy chain variable region (VH), wherein the VL comprises an amino acid sequence as set forth in SEQ ID NO: 117, and the VH comprises an amino acid sequence as set forth in SEQ ID NO: 107.

In some specific embodiments, the PD-L1 antibody or the antigen-binding fragment thereof comprises a light chain having a sequence as set forth in SEQ ID NO: 136 and a heavy chain having a sequence as set forth in SEQ ID NO: 126.

In some specific embodiments, the B7H4 antibody or the antigen-binding fragment thereof comprises a light chain having a sequence as set forth in SEQ ID NO: 139 and a heavy chain having a sequence as set forth in SEQ ID NO: 131.

In some specific embodiments, the 4-1BB antibody or the antigen-binding fragment thereof comprises a light chain having a sequence as set forth in SEQ ID NO: 134 and a heavy chain having a sequence as set forth in SEQ ID NO: 124.

In some specific embodiments, the 4-1BB antibody or the antigen-binding fragment thereof comprises a heavy chain having a sequence as set forth in SEQ ID NO: 129.

In some specific embodiments, the OX40 antibody or the antigen-binding fragment thereof comprises a heavy chain having a sequence as set forth in SEQ ID NO: 130.

In some specific embodiments, the BCMA antibody or the antigen-binding fragment thereof comprises a heavy chain having a sequence as set forth in SEQ ID NO: 133.

In some specific embodiments, the BCMA antibody or the antigen-binding fragment thereof comprises a light chain having a sequence as set forth in SEQ ID NO: 138 and a heavy chain having a sequence as set forth in SEQ ID NO: 128.

In some specific embodiments, the CTLA4 antibody or the antigen-binding fragment thereof comprises a heavy chain having a sequence as set forth in SEQ ID NO: 123.

In some specific embodiments, the HER2 antibody or the antigen-binding fragment thereof comprises a light chain having a sequence as set forth in SEQ ID NO: 135 and a heavy chain having a sequence as set forth in SEQ ID NO: 125.

In some specific embodiments, the binding protein comprises two protein functional regions: a protein functional region A and a protein functional region B, wherein the protein functional region A comprises a light chain variable region (VL) and a heavy chain variable region (VH), wherein the VL comprises LCDR1, LCDR2 and LCDR3 with amino acid sequences as set forth in SEQ ID NOs: 75, 85 and 97, respectively; and the VH comprises HCDR1, HCDR2 and HCDR3 with amino acid sequences as set forth in SEQ ID NOs: 13, 32 and 54, respectively; and, the protein functional region B comprises a heavy chain variable region (VH), wherein the VH comprises HCDR1, HCDR2 and HCDR3 with amino acid sequences as set forth in SEQ ID NOs: 14, 35 and 57, respectively. The amino acid sequences of the listed CDRs are shown according to the Chothia scheme.

In some specific embodiments, the binding protein comprises two protein functional regions: a protein functional region A and a protein functional region B, wherein the protein functional region A comprises a light chain variable region (VL) and a heavy chain variable region (VH), wherein the VL comprises LCDR1, LCDR2 and LCDR3 with amino acid sequences as set forth in SEQ ID NOs: 78, 83 and 100, respectively; and the VH comprises HCDR1, HCDR2 and HCDR3 with amino acid sequences as set forth in SEQ ID NOs: 15, 37 and 59, respectively; and, the protein functional region B comprises a heavy chain variable region (VH), wherein the VH comprises HCDR1, HCDR2 and HCDR3 with amino acid sequences as set forth in SEQ ID NOs: 14, 35 and 57, respectively. The amino acid sequences of the listed CDRs are shown according to the Chothia scheme.

In some specific embodiments, the binding protein comprises two protein functional regions: a protein functional region A and a protein functional region B, wherein the protein functional region A comprises a light chain variable region (VL) and a heavy chain variable region (VH), wherein the VL comprises LCDR1, LCDR2 and LCDR3 with amino acid sequences as set forth in SEQ ID NOs: 78, 83 and 100, respectively; and the VH comprises HCDR1, HCDR2 and HCDR3 with amino acid sequences as set forth in SEQ ID NOs: 15, 37 and 59, respectively; and, the protein functional region B comprises a heavy chain variable region (VH), wherein the VH comprises HCDR1, HCDR2 and HCDR3 with amino acid sequences as set forth in SEQ ID NOs: 13, 36 and 58, respectively. The amino acid sequences of the listed CDRs are shown according to the Chothia scheme.

In some specific embodiments, the binding protein comprises two protein functional regions: a protein functional region A and a protein functional region B, wherein the protein functional region A comprises a light chain variable region (VL) and a heavy chain variable region (VH), wherein the VL comprises LCDR1, LCDR2 and LCDR3 with amino acid sequences as set forth in SEQ ID NOs: 77, 87 and 99, respectively; and the VH comprises HCDR1, HCDR2 and HCDR3 with amino acid sequences as set forth in SEQ ID NOs: 13, 34 and 56, respectively; and, the protein functional region B comprises a heavy chain variable region (VH), wherein the VH comprises HCDR1, HCDR2 and HCDR3 with amino acid sequences as set forth in SEQ ID NOs: 17, 39 and 61, respectively. The amino acid sequences of the listed CDRs are shown according to the Chothia scheme.

In some specific embodiments, the binding protein comprises two protein functional regions: a protein functional region A and a protein functional region B, wherein the protein functional region A comprises a light chain variable region (VL) and a heavy chain variable region (VH), wherein the VL comprises LCDR1, LCDR2 and LCDR3 with amino acid sequences as set forth in SEQ ID NOs: 74, 84 and 96, respectively; and the VH comprises HCDR1, HCDR2 and HCDR3 with amino acid sequences as set forth in SEQ ID NOs: 12, 31 and 53, respectively; and, the protein functional region B comprises a heavy chain variable region (VH), wherein the VH comprises HCDR1, HCDR2 and HCDR3 with amino acid sequences as set forth in SEQ ID NOs: 10, 29 and 51, respectively. The amino acid sequences of the listed CDRs are shown according to the Chothia scheme.

In some specific embodiments, the binding protein comprises two protein functional regions: a protein functional region A and a protein functional region B, wherein the protein functional region A comprises a light chain variable region with an amino acid sequence as set forth in SEQ ID NO: 118 and a heavy chain variable region with an amino acid sequence as set forth in SEQ ID NO: 108; and the protein functional region B comprises a heavy chain variable region with an amino acid sequence as set forth in SEQ ID NO: 111.

In some specific embodiments, the binding protein comprises two protein functional regions: a protein functional region A and a protein functional region B, wherein the protein functional region A comprises a light chain variable region with an amino acid sequence as set forth in SEQ ID NO: 121 and a heavy chain variable region with an amino acid sequence as set forth in SEQ ID NO: 113; and the protein functional region B comprises a heavy chain variable region with an amino acid sequence as set forth in SEQ ID NO: 111.

In some specific embodiments, the binding protein comprises two protein functional regions: a protein functional region A and a protein functional region B, wherein the protein functional region A comprises a light chain variable region with an amino acid sequence as set forth in SEQ ID NO: 121 and a heavy chain variable region with an amino acid sequence as set forth in SEQ ID NO: 113; and the protein functional region B comprises a heavy chain variable region with an amino acid sequence as set forth in SEQ ID NO: 112.

In some specific embodiments, the binding protein comprises two protein functional regions: a protein functional region A and a protein functional region B, wherein the protein functional region A comprises a light chain variable region with an amino acid sequence as set forth in SEQ ID NO: 120 and a heavy chain variable region with an amino acid sequence as set forth in SEQ ID NO: 110; and the protein functional region B comprises a heavy chain variable region with an amino acid sequence as set forth in SEQ ID NO: 115.

In some specific embodiments, the binding protein comprises two protein functional regions: a protein functional region A and a protein functional region B, wherein the protein functional region A comprises a light chain variable region with an amino acid sequence as set forth in SEQ ID NO: 117 and a heavy chain variable region with an amino acid sequence as set forth in SEQ ID NO: 107; and the protein functional region B comprises a heavy chain variable region with an amino acid sequence as set forth in SEQ ID NO: 105.

In some specific embodiments, the binding protein comprises two polypeptide chains: a first polypeptide chain and a second polypeptide chain, wherein the first polypeptide chain comprises an amino acid sequence as set forth in SEQ ID NO: 147; and the second polypeptide chain comprises an amino acid sequence as set forth in SEQ ID NO: 153.

In some specific embodiments, the binding protein comprises two polypeptide chains: a first polypeptide chain and a second polypeptide chain, wherein the first polypeptide chain comprises an amino acid sequence as set forth in SEQ ID NO: 136; and the second polypeptide chain comprises an amino acid sequence as set forth in SEQ ID NO: 183.

In some specific embodiments, the binding protein comprises two polypeptide chains: a first polypeptide chain and a second polypeptide chain, wherein the first polypeptide chain comprises an amino acid sequence as set forth in SEQ ID NO: 147; and the second polypeptide chain comprises an amino acid sequence as set forth in SEQ ID NO: 184.

In some specific embodiments, the binding protein comprises two polypeptide chains: a first polypeptide chain and a second polypeptide chain, wherein the first polypeptide chain comprises an amino acid sequence as set forth in SEQ ID NO: 155; and the second polypeptide chain comprises an amino acid sequence as set forth in SEQ ID NO: 158.

In some specific embodiments, the binding protein comprises two polypeptide chains: a first polypeptide chain and a second polypeptide chain, wherein the first polypeptide chain comprises an amino acid sequence as set forth in SEQ ID NO: 155; and the second polypeptide chain comprises an amino acid sequence as set forth in SEQ ID NO: 156.

In some specific embodiments, the binding protein comprises two polypeptide chains: a first polypeptide chain and a second polypeptide chain, wherein the first polypeptide chain comprises an amino acid sequence as set forth in SEQ ID NO: 159; and the second polypeptide chain comprises an amino acid sequence as set forth in SEQ ID NO: 160.

In some specific embodiments, the binding protein comprises two polypeptide chains: a first polypeptide chain and a second polypeptide chain, wherein the first polypeptide chain comprises an amino acid sequence as set forth in SEQ ID NO: 141; and the second polypeptide chain comprises an amino acid sequence as set forth in SEQ ID NO: 142.

In some specific embodiments, the binding protein comprises two polypeptide chains: a first polypeptide chain and a second polypeptide chain, wherein the first polypeptide chain comprises an amino acid sequence as set forth in SEQ ID NO: 141; and the second polypeptide chain comprises an amino acid sequence as set forth in SEQ ID NO: 143.

In some specific embodiments, the binding protein comprises two polypeptide chains: a first polypeptide chain and a second polypeptide chain, wherein the first polypeptide chain comprises an amino acid sequence as set forth in SEQ ID NO: 141; and the second polypeptide chain comprises an amino acid sequence as set forth in SEQ ID NO: 144.

In some specific embodiments, the binding protein comprises two polypeptide chains: a first polypeptide chain and a second polypeptide chain, wherein the first polypeptide chain comprises an amino acid sequence as set forth in SEQ ID NO: 141; and the second polypeptide chain comprises an amino acid sequence as set forth in SEQ ID NO: 145.

In some specific embodiments, the binding protein comprises two polypeptide chains: a first polypeptide chain and a second polypeptide chain, wherein the first polypeptide chain comprises an amino acid sequence as set forth in SEQ ID NO: 141; and the second polypeptide chain comprises an amino acid sequence as set forth in SEQ ID NO: 149.

In the present application, the CDRs may comprise mutations based on the defined sequences. The mutation is an insertion, deletion or substitution of 3, 2 or 1 amino acids on the basis of the amino acid sequences of the VH CDR1, the VH CDR2, the VH CDR3, the VL CDR1, the VL CDR2 and the VL CDR3. In the present application, “amino acid mutation” in the context like “insertion, deletion or substitution of 3, 2 or 1 amino acids” refers to a mutation of an amino acid in the sequence of a variant as compared to the sequence of an original amino acid, including the insertion, deletion or substitution of amino acids on the basis of the original amino acid sequence. An exemplary explanation is that the mutations to the CDRs may comprise 3, 2 or 1 amino acid mutations, and that the same or different numbers of amino acid residues can be optionally selected for the mutations to those CDRs, e.g., 1 amino acid mutation to CDR1, and no amino acid mutation to CDR2 and CDR3.

In the present application, the VH and VL or the polypeptide chain may comprise mutations based on the defined sequences. The mutation is a deletion, substitution or addition of one or more amino acid residues on the defined amino acid sequence, and the amino acid sequence with the mutation has at least 85% sequence identity to the defined amino acid sequence and maintains or improves the binding activity of the antibody or the antigen-binding fragment thereof and the binding protein, wherein the at least 85% sequence identity is preferably at least 90% sequence identity, more preferably at least 95% sequence identity, and most preferably at least 99% sequence identity.

In order to solve the above technical problems, a second aspect of the present invention provides an isolated nucleic acid encoding the binding protein according to the first aspect of the present invention.

In order to solve the above technical problems, a third aspect of the present invention provides a recombinant expression vector comprising the isolated nucleic acid according to the second aspect of the present invention. Preferably, the expression vector comprises a eukaryotic cell expression vector and/or a prokaryotic cell expression vector.

In order to solve the above technical problems, a fourth aspect of the present invention provides a transformant comprising the isolated nucleic acid according to the second aspect of the present invention or the recombinant expression vector according to the third aspect of the present invention. Preferably, the transformant has a host cell being a prokaryotic cell and/or a eukaryotic cell, wherein the prokaryotic cell is preferably an E. coli cell such as TG1 and BL21, and the eukaryotic cell is preferably an HEK293 cell or a CHO cell.

In order to solve the above technical problems, a fifth aspect of the present invention provides a method for preparing a binding protein, which comprises culturing the transformant according to the fourth aspect of the present invention, and obtaining the binding protein from a culture.

In order to solve the above technical problems, a sixth aspect of the present invention provides a pharmaceutical composition comprising the binding protein according to the first aspect of the present invention, and a pharmaceutically acceptable carrier. preferably, the pharmaceutical composition further comprises an additional anti-tumor antibody as an active ingredient.

In order to solve the above technical problems, a seventh aspect of the present invention provides a kit comprising the binding protein according to the first aspect of the present invention and/or the pharmaceutical composition according to the sixth aspect of the present invention.

Preferably, the kit further comprises (i) a device for administering the binding protein or the pharmaceutical composition; and/or (ii) instructions for use.

In order to solve the above technical problems, an eighth aspect of the present invention provides a combination of kits comprising a kit I and a kit II, wherein the kit I comprises the binding protein according to the first aspect of the present invention and/or the pharmaceutical composition according to the sixth aspect of the present invention, and the kit II comprises an additional antibody or pharmaceutical composition.

In order to solve the above technical problems, a ninth aspect of the present invention provides an administration device comprising the binding protein according to the first aspect of the present invention and/or the pharmaceutical composition according to the sixth aspect of the present invention.

Preferably, the administration device further comprises a component, such as a syringe, an infusion device or an implantable administration device, for containing or administering the binding protein and/or the pharmaceutical composition to a subject.

In order to solve the above technical problems, a tenth aspect of the present invention provides use of the binding protein according to the first aspect of the present invention, the pharmaceutical composition according to the sixth aspect of the present invention, the kit according to the seventh aspect of the present invention, the combination of kits according to the eighth aspect of the present invention, and/or the administration device according to the ninth aspect of the present invention in the preparation of a medicament for the diagnosis, prevention and/or treatment of cancer or other diseases.

Preferably, the cancer is selected from one or more of breast cancer, ovarian cancer, endometrial cancer, renal cancer, melanoma, lung cancer, gastric cancer, liver cancer, esophageal cancer, cervical cancer, head and neck neoplasm, cholangiocarcinoma, gallbladder cancer, bladder cancer, sarcoma, colorectal cancer, lymphoma and multiple myeloma.

In order to solve the above technical problems, an eleventh aspect of the present invention provides a method for detecting a specific antigen in vitro or in vivo comprising, which comprises detecting with the binding protein according to the first aspect of the present invention and/or the pharmaceutical composition according to the sixth aspect of the present invention.

In order to solve the above technical problems, a twelfth aspect of the present invention provides use of the binding protein according to the first aspect of the present invention, the pharmaceutical composition according to the sixth aspect of the present invention, the kit according to the seventh aspect of the present invention, the combination of kits according to the eighth aspect of the present invention, and/or the administration device according to the ninth aspect of the present invention in the diagnosis, prevention and/or treatment of cancer or other diseases.

Preferably, the cancer is selected from one or more of breast cancer, ovarian cancer, endometrial cancer, renal cancer, melanoma, lung cancer, gastric cancer, liver cancer, esophageal cancer, cervical cancer, head and neck neoplasm, cholangiocarcinoma, gallbladder cancer, bladder cancer, sarcoma, colorectal cancer, lymphoma and multiple myeloma.

In order to solve the above technical problems, a thirteenth aspect of the present invention provides a method for diagnosing, preventing and/or treating cancer or other diseases, which comprises the step of administering to a patient in need thereof the binding protein according to the first aspect of the present invention, the pharmaceutical composition according to the sixth aspect of the present invention, the kit according to the seventh aspect of the present invention, the combination of kits according to the eighth aspect of the present invention, and/or the administration device according to the ninth aspect of the present invention.

Preferably, the cancer is selected from one or more of breast cancer, ovarian cancer, endometrial cancer, renal cancer, melanoma, lung cancer, gastric cancer, liver cancer, esophageal cancer, cervical cancer, head and neck neoplasm, cholangiocarcinoma, gallbladder cancer, bladder cancer, sarcoma, colorectal cancer, lymphoma and multiple myeloma.

On the basis of the general knowledge in the art, the above preferred conditions can be combined arbitrarily to obtain preferred embodiments of the present invention.

The reagents and starting materials used in the present invention are commercially available.

The beneficial effects of the present invention are as follows:

The present invention provides a bispecific binding protein with a Fab-HCAb structure constructed using a heavy-chain antibody (HCAb) and an antigen-binding region Fab of a conventional antibody. The bispecific binding protein molecule with the Fab-HCAb structure of the present invention has a simple and universal structure, and can be suitable for various target combinations. It has the characteristics of a relatively small molecular weight, less polypeptide chains, simple structure and the like, and also has the similar Fc effector function to the IgG antibody, excellent molecular stability and pharmaceutical properties, and the like. Moreover, it is more advantageous than existing bispecific binding proteins with other structures.

In a certain preferred embodiment, the molecule with the Fab-HCAb structure has one or more of the following advantages over a molecule with an FIT-Ig structure, a VH-IgG structure or an IgG-VH structure:
(1) the Fab-HCAb structure has a relatively small molecular weight, with only two different polypeptide chains, so that it is a simpler structure and produces almost no mismatch of polypeptide chains;
(2) the Fab-HCAb structure has better target-binding ability;
(3) the distance between the first binding domain (Fab) and the second binding domain (VH) of the Fab-HCAb structure is more favorable for the interaction between a target cell (e.g., a tumor cell) and an effector cell (e.g., a T cell) to form an immune synapse to further promote activation of the effector cell;
(4) the Fab-HCAb structure has stronger effector cell activation ability;
(5) the molecule with the Fab-HCAb structure can be free of additional linker peptides, thereby reducing the risk of being cleaved due to the linker peptides;
(6) the Fab-HCAb structure is more compact and has a closer distance between its two second binding domains (VH), which in some cases is more favorable for clustering and polymerization of targets; and
(7) the Fab-HCAb structure may preferentially bind to the target recognized by the Fab domain before causing the binding of the VH domain, and the order of binding and the difference in binding force of different targets may be suitable for the requirements of some special application scenarios, for example, the Fab-HCAb of TAA×4-1BB may preferentially bind to tumor targets.

### BRIEF DESCRIPTION OF THE DRAWINGS

FIG. 1 shows the schematic diagrams of molecular structures.

FIG. 2 shows the binding activity of PD-L1×4-1BB molecules to human 4-1BB cells CHO-K1/hu 4-1BB.

FIG. 3 shows the binding activity of PD-L1×4-1BB molecules to human PD-L1 cells CHO-K1/hPD-L1.

FIG. 4 shows the activation of PD-L1×4-1BB molecules on T cells in a mixed lymphocyte reaction (MLR) assay: (A) IL-2 release level; (B) IFN-γ release level.

FIG. 5 shows the binding activity of B7H4×4-1BB molecules to human 4-1BB cells CHO-K1/hu 4-1BB.

FIG. 6 shows the binding activity of B7H4×4-1BB molecules to tumor cells SK-BR-3.

FIG. 7 shows the T cell specific activation mediated by B7H4×4-1BB molecules in SK-BR-3 cells.

FIG. 8 shows the T cell specific activation mediated by PD-L1×4-1BB molecules in CHO-K1/hPD-L1 cells.

FIG. 9 shows the binding activity of B7H4×OX40 molecules to human OX40 cells CHO-K1/hu OX40.

FIG. 10 shows the binding activity of B7H4×OX40 molecules to tumor cells SK-BR-3.

FIG. 11 shows the T cell specific activation mediated by B7H4×OX40 molecules by human B7H4 cells CHO-K1/hB7H4.

FIG. 12 shows the internalization of BCMA-binding proteins on NCI-H929 cells.

FIG. 13 shows the determination of affinity of BCMA-binding proteins for BCMA by a BLI method: (A) heavy-chain antibody PR004433; (B) a bispecific binding protein of a Fab-HCAb structure PR005744.

FIG. 14 shows the binding activity of HER2×CTLA4 molecules to tumor cells SK-BR-3.

FIG. 15 shows the binding activity of HER2×CTLA4 molecules to human CTLA4 cells CHO-K1/hCTLA4.

FIG. 16 shows the pharmacokinetics of a molecule of a Fab-HCAb structure PR004270 in mice.

FIG. 17 shows predicted Fab-HCAb structures: (A) a three-dimensional structure model of Fab-HCAb, in which A1 and A2 are antigen-binding sites at the Fab end, and B1 and B2 are antigen-binding sites at the VH end; (B) relative distances between different antigen-binding sites when the Fab-HCAb structure is in its most extended state; (C) relative distances between different antigen-binding sites when the FIT-Ig structure is in its most extended state.

### DETAILED DESCRIPTION

The embodiments of the present invention are described below with reference to specific examples, and other advantages and effects of the present invention will be readily apparent to those skilled in the art from the disclosure of the present specification.

In the present application, the term “binding protein” or “antigen-binding protein” generally refers to a protein comprising an antigen-binding moiety, and optionally a scaffold or framework moiety that allows the antigen-binding moiety to adopt a conformation that facilitates the binding of the antigen-binding protein to the antigen. An antibody may typically comprise an antibody light chain variable region (VL) or an antibody heavy chain variable region (VH), or both. The VH and VL regions can be further divided into hypervariable regions termed complementarity determining regions (CDRs), which are scattered over more conserved regions termed framework regions (FRs). Each VH and VL can consist of three CDR regions and four FR regions arranged from amino-terminus to carboxyl-terminus in the following order: FR-1, CDR1, FR-2, CDR2, FR-3, CDR3 and FR-4. The variable regions of the heavy and light chains comprise binding domains that interact with antigens. The three CDRs of VH are denoted as HCDR1, HCDR2 and HCDR3, respectively, and may also be denoted as VH CDR1, VH CDR2 and VH CDR3, respectively; and the three CDRs of VL are denoted as LCDR1, LCDR2 and LCDR3, respectively, and may also be denoted as VL CDR1, VL CDR2 and VL CDR3, respectively. Examples of the antigen-binding proteins include, but are not limited to, antibodies, antigen-binding fragments (Fab, Fab′, F(ab)₂, Fv fragment, F(ab′)2, scFv, di-scFv and/or dAb), immunoconjugates, multispecific antibodies (e.g., bispecific antibodies), antibody fragments, antibody derivatives, antibody analogs or fusion proteins, as long as they exhibit the desired antigen-binding activity.

In the present application, the amino acid sequences of the CDRs are shown according to the Chothia scheme. However, it is well known to those skilled in the art that the CDRs of an antibody can be defined in the art using a variety of methods, such as the Kabat scheme based on sequence variability (see Kabat et al., Sequences of Proteins of Immunological Interest, Fifth Edition, National Institutes of Health (U.S.), Bethesda, Md. (1991)), and the Chothia scheme based on the location of the structural loop regions (see J Mol Biol 273: 927-948, 1997). In the technical solution of the present invention, the Combined scheme comprising the Kabat scheme and the Chothia scheme can also be used to determine the amino acid residues in a variable domain sequence. The Combined scheme combines the Kabat scheme with the Chothia scheme to obtain a larger range. See the table below for details. It will be understood by those skilled in the art that unless otherwise specified, the terms “CDR” and “complementarity determining region” of a given antibody or a region (e.g., variable region) thereof are construed as encompassing complementary determining regions as defined by any one of the above known schemes described herein. Although the scope claimed in the present invention is the sequences shown based on the Chothia scheme, the amino acid sequences corresponding to the other schemes for numbering CDRs shall also fall within the scope of the present invention.

TABLE I
The schemes for numbering the CDRs of
the antibody of the present application

| | Kabat | Chothia | Combined |
|---|---|---|---|
| LCDR1 | L24--L34 | L24--L34 | L24-L34 |
| LCDR2 | L50--L56 | L50--L56 | L50-L56 |
| LCDR3 | L89--L97 | L89--L97 | L89-L97 |
| HCDR1 | H31--H35 | H26--H32 | H26-H35 |
| HCDR2 | H50--H65 | H52--H56 | H50-H65 |
| HCDR3 | H95--H102 | H95--H102 | H95-H102 |

Laa-Lbb can refer to an amino acid sequence from position aa (the Chothia scheme) to position bb (the Chothia scheme) beginning at the N-terminus of the light chain of the antibody; and Haa-Hbb can refer to an amino acid sequence from position aa (the Chothia scheme) to position bb (the Chothia scheme) beginning at the N-terminus of the heavy chain of the antibody. For example, L24-L34 can refer to the amino acid sequence from position 24 to position 34 according to the Chothia scheme beginning at the N-terminus of the light chain of the antibody; H26-H32 can refer to the amino acid sequence from position 26 to position 32 according to the Chothia scheme beginning at the N-terminus of the heavy chain of the antibody. It should be known to those skilled in the art that there are positions where insertion sites are present in numbering CDRs with the Chothia scheme (see http://bioinf.org.uk/abs/).

In the present application, the term “monoclonal antibody” generally refers to an antibody obtained from a population of substantially homogeneous antibodies, that is, the individual antibodies in the population are identical except for a small number of natural mutations that may exist. Monoclonal antibodies are generally highly specific for a single antigenic site. Moreover, unlike conventional polyclonal antibody preparations (which generally have different antibodies directed against different determinants), each monoclonal antibody is directed against a single determinant on the antigen. In addition to their specificity, monoclonal antibodies have the advantage that they can be synthesized by hybridoma culture without contamination by other immunoglobulins. The modifier “monoclonal” indicates the characteristic of the antibody obtained from a population of substantially homogeneous antibodies, and is not to be construed as requiring production of the antibody by any particular method. For example, monoclonal antibodies used according to the present invention can be prepared in hybridoma cells or can be prepared by the recombinant DNA method.

In the present application, the term “fully human antibody” generally refers to an antibody that is expressed by a genetically engineered antibody gene-deleted animal into which the entire or part of gene that encode an antibody in human is transferred. All parts of the antibody (including the variable and constant regions of the antibody) are encoded by genes of human origin. The fully human antibody can greatly reduce the immune side effects caused in the human body by the heterologous antibody. Methods for obtaining fully human antibodies in the art can include phage display, transgenic mice, and the like.

In the present application, the term “specifically bind to” generally refers to that an antibody binds to an epitope via its antigen-binding domain, and that the binding requires some complementarity between the antigen-binding domain and the epitope. According to this definition, an antibody is said to “specifically bind to” an antigen when the antibody more easily binds to an epitope via its antigen-binding domain than binds to a random, unrelated epitope. “Epitope” refers to a specific atomic group (e.g., saccharide side chain, phosphoryl, sulfonyl) or an amino acid on an antigen that binds to an antigen-binding protein (e.g., an antibody).

In the present application, the term “Fab” generally refers to the portion of a conventional antibody (e.g., IgG) that binds to an antigen, including the heavy chain variable region VH, the light chain variable region VL, the heavy chain constant region domain CH1 and the light chain constant region CL of the antibody. In conventional antibodies, the C-terminus of VH is linked to the N-terminus of CH1 to form a heavy chain Fd fragment, the C-terminus of VL is linked to the N-terminus of CL to form a light chain, and the C-terminus of CH1 is further linked to the hinge region and other constant region domains of the heavy chain to form a heavy chain. In some embodiments, “Fab” also refers to a variant structure of the Fab. For example, in certain embodiments, the C-terminus of VH is linked to the N-terminus of CL to form one polypeptide chain, and the C-terminus of VL is linked to the N-terminus of CH1 to form the other polypeptide chain, in which case an Fab (cross VH/VL) structure is formed; in certain embodiments, CH1 of the Fab is not linked to the hinge region, but rather the C-terminus of CL is linked to the hinge region of the heavy chain, in which case a Fab (cross Fd/LC) structure is formed.

In the present application, the term “VH” generally refers to the heavy chain variable region VH domain of an antibody, i.e., the heavy chain variable region VH of a conventional antibody (H2L2 structure) from human or other animals, the heavy chain variable region VHH of a heavy-chain antibody (HCAb structure) from animals such as those of Camelidae species, or the heavy chain variable region VH of a fully human heavy-chain antibody (HCAb structure) produced using a Harbour HCAb transgenic mouse.

In the present application, the term “antigen-binding fragment” generally refers to any protein functional region that can specifically bind to the antigen, either “Fab” or “VH”, or other antigen-binding forms (e.g., derived protein structures such as lipocalins, neuronal cell adhesion molecules (NCAMs), fibronectins, and designed ankyrin repeat proteins (DARPins)).

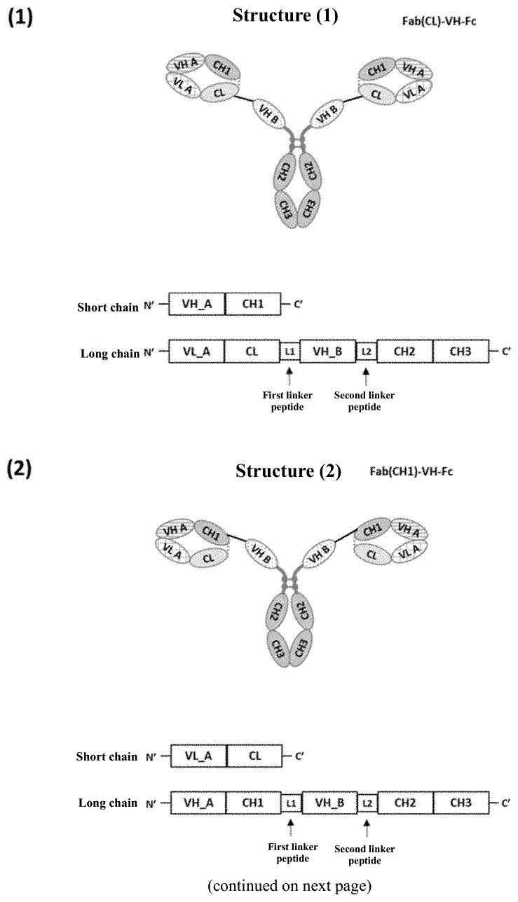

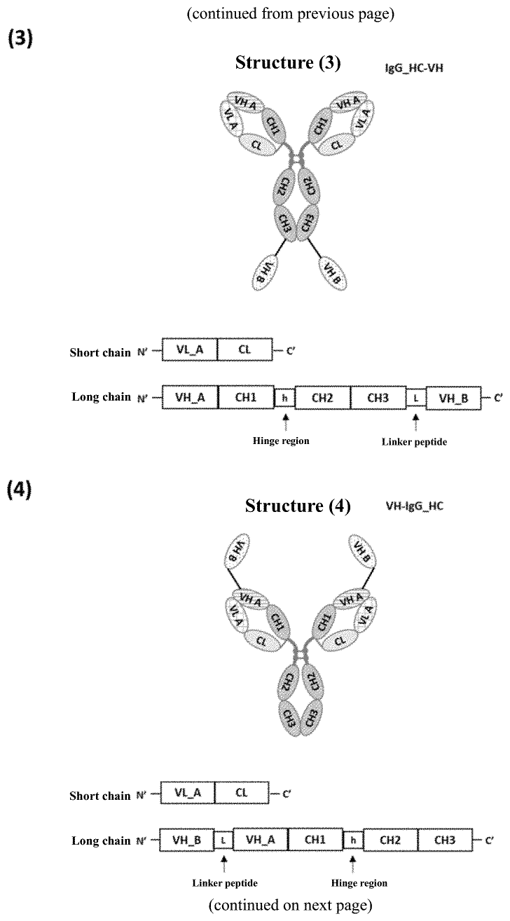

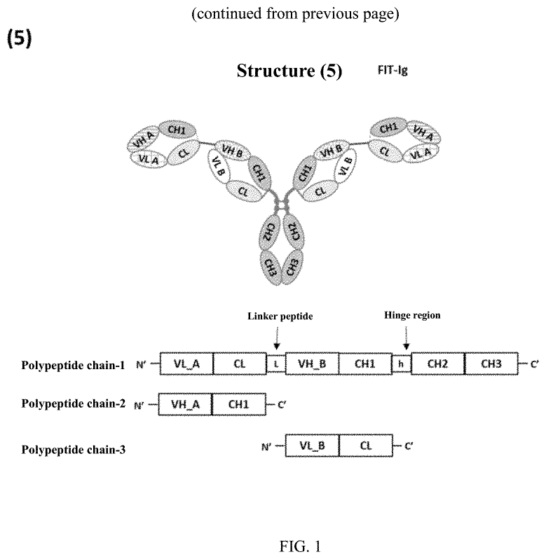

In the present application, the term “Fab-HCAb structure” is a structure shown as structure (1) or structure (2) in Table 1 and FIG. 1. The structure comprises two polypeptide chains: a polypeptide chain 1, also known as a short chain, comprising VH_A-CH1 from the amino-terminus to the carboxyl-terminus; and a polypeptide chain 2, also known as a long chain, comprising VL_A-CL-L1-VH_B-L2-CH2-CH3 from the amino-terminus to the carboxyl-terminus. Alternatively, the structure may also comprise two polypeptide chains: a polypeptide chain 1, also known as a short chain, comprising VL_A-CL from the amino-terminus to the carboxyl-terminus; and a polypeptide chain 2, also known as a long chain, comprising VH_A-CH1-L1-VH_B-L2-CH2-CH3 from the amino-terminus to the carboxyl-terminus. VH_A and VL_A are heavy chain and light chain variable regions of a conventional antibody A, respectively; VH_B is a heavy chain variable region of a heavy-chain antibody B; CL is a domain of a light chain constant region; CH1, CH2 and CH3 are first, second and third domains of a heavy chain constant region, respectively; and L1 and L2 are linker peptides. In certain embodiments, L1 may be 0 in length. In certain embodiments, L2 may be a hinge region or a hinge region-derived linker peptide sequence of an IgG, or a sequence listed in Table 2. In certain embodiments, the “Fab-HCAb structure” refers specifically to the form of structure (1).

In the present application, the term “tumor antigen” may be either a tumor specific antigen (TSA) or a tumor-associated antigen (TAA). Tumor specific antigen refers to an antigen that is specific to tumor cells and is not present in normal cells or tissues. Tumor-associated antigen is not specific to tumor cells and is also present in normal cells or tissues, but is highly expressed when tumor cells proliferate.

In the present application, the term “target cell” refers to a cell that needs to be eliminated, mainly a tumor cell, and may be an immunosuppressive cell or the like.

In the present application, the term “effector cell” generally refers to an immune cell involved in the clearance of foreign antigens and performing effector functions in an immune response. e.g., a plasma cell, a cytotoxic T cell, a NK cell, and the like.

In the present application, the term “PD-L1” generally refers to the programmed death ligand 1 protein, a functional variant thereof and/or a functional fragment thereof. PD-L1 is also known as cluster of differentiation 274 (CD274) or B7 homologue 1 (B7-H1), and is a protein encoded by the CD274 gene (in human). The sequence of PD-L1 is known in the art. For example, the amino acid sequence of an exemplary full-length human PD-L1 protein can be found under NCBI accession No. NP_054862 or UniProt accession No. Q9NZQ7; and the sequence of an exemplary full-length cynomolgus monkey PD-L1 protein can be found under NCBI accession No. XP_005581836 or Uniprot accession No. G7PSE7. PD-L1 is mainly expressed in antigen-presenting cells and a variety of tumor cells. The interaction between PD-L1 and PD-1 can down-regulate the activity of T cells, weaken the secretion of cytokines and play a role in immunosuppression. The expression of the PD-L1 protein can be detected in many human tumor tissues. The microenvironment at the tumor site can induce the expression of PD-L1 on tumor cells, and the expressed PD-L1 facilitates the occurrence and growth of tumors, induces the apoptosis of anti-tumor T cells and further protects the tumor cells from immune attack.

In the present application, the term “HER2” generally refers to the receptor tyrosine kinase erbB-2 (also known as ERBB2), a functional variant thereof and/or a functional fragment thereof. The sequence of HER2 is known in the art. For example, the sequence of an exemplary full-length human HER2 can be found under Uniprot accession No. P04626; and the sequence of an exemplary full-length cynomolgus monkey HER2 can be found under NCBI accession No. XP_005584091.

In the present application, the term “B7H4” generally refers to the V-Set domain-containing T-cell activation inhibitor 1 (also known as VTCN1, B7h.5, B7S1 or B7x), a functional variant thereof and/or a functional fragment thereof. The sequence of B7H4 is known in the art. For example, the sequence of an exemplary full-length human B7H4 can be found under Uniprot accession No. Q7Z7D3; the sequence of an exemplary full-length cynomolgus monkey B7H4 can be found under NCBI accession No. XP_005542249; and the sequence of an exemplary full-length mouse B7H4 can be found under Uniprot accession No. Q7TSP5. B7-H4 is a transmembrane protein belonging to the B7/CD28 superfamily. The B7-H4 protein is expressed in some immune cells such as monocytes and dendritic cells, and is possibly involved in the negative regulation of immune response of T cells. In addition, B7H4 is highly expressed on the surface of tumor cells of breast cancer, ovarian cancer, endometrial cancer, non-small cell lung cancer, kidney cancer, etc., while it is not expressed or is very little expressed in most normal tissues. As an emerging target for these tumors, B7-H4 has received attention in recent years. Anti-B7-H4 antibodies can act on tumor cells through multiple mechanisms, but its development is mainly focused on monoclonal antibodies, and no bispecific antibody therapy is available at present.

In the present application, the term “4-1BB” generally refers to the tumor necrosis factor receptor superfamily member 9 (also known as CD137, TNFRSF9 or 4-1BBL receptor), a functional variant thereof and/or a functional fragment thereof. The sequence of 4-1BB is known in the art. For example, the sequence of an exemplary full-length human 4-1BB can be found under Uniprot accession No. Q07011; and the sequence of an exemplary full-length cynomolgus monkey 4-1BB can be found under NCBI accession No. XP_005544945. 4-1BB is a transmembrane protein belonging to the TNF receptor superfamily. 4-1BB is a costimulatory molecule expressed on a variety of immune cells. It is a multifunctional modulator of immune activity. Its expression is induced in activated T cells, NK cells and other immune cells. 4-1BB activates T cells through trimerization mediated by its ligand 4-1BBL, thereby promoting cell proliferation and cytokine release. Anti-4-1BB agonistic antibodies have the function of inhibiting tumors. The first 4-1BB antibodies to be subjected to clinical trials were Utomilumab from Pfizer and Urelumab (BMS-663513) from Bristol-Myers Squibb (BMS). The initial clinical results of Urelumab were published in 2008. Although encouraging efficacy was observed in some patients, the data showed Urelumab to cause target and dose-associated hepatotoxicity. Utomilumab has better safety enabling the dose to be increased to 10 mg/kg, but still has a poor therapeutic effect. The core problem of the development of 4-1BB-targeted drugs is how to properly activate immune cells through 4-1BB to achieve a balance between efficacy and safety.

In the present application, the term “OX40” generally refers to the tumor necrosis factor receptor superfamily member 4 (also known as CD134, TNFRSF4 or OX40L receptor), a functional variant thereof and/or a functional fragment thereof. The sequence of OX40 is known in the art. For example, the sequence of an exemplary full-length human OX40 can be found under Uniprot accession No. P43489; and the sequence of an exemplary full-length cynomolgus monkey OX40 can be found under NCBI accession No. XP_005545179. OX40, one of the TNF receptor superfamily members, is involved in enhancing T cell receptor-triggered T cell responses, and is a costimulatory receptor molecule. It is a transmembrane protein of 50 kD. OX40 is transiently expressed on human CD4⁺ and CD8⁺ T cells after TCR stimulation. However, at the tumor site, OX40 is more highly expressed on CD4⁺ T cells than on CD8⁺ T cells. Thus, CD4⁺ and CD8⁺ T cells are potential targets of OX40-directed immunotherapy of cancer. Some preclinical studies on OX40 antibodies have shown that anti-OX40 monoclonal antibodies produce deleterious immunosuppressive side effects by promoting MDSC accumulation and Th2 cytokine production.

In the present application, the term “BCMA” refers generally to the tumor necrosis factor receptor superfamily member 17 (also known as B-cell maturation antigen, TNFRSF17 or CD269), a functional variant thereof and/or a functional fragment thereof. The sequence of BCMA is known in the art. For example, the sequence of an exemplary full-length human BCMA can be found under Uniprot accession No. Q02223; and the sequence of an exemplary full-length cynomolgus monkey BCMA can be found under NCBI accession No.

XP_005591343. BCMA is a transmembrane protein belonging to the TNF receptor superfamily that is involved in B cell maturation, growth and survival. BCMA has two major ligands: the high-affinity ligand APRIL and the low-affinity ligand BAFF. BCMA is expressed in malignant plasma cells of multiple myeloma (MM) patients and supports the growth and survival of multiple myeloma cells. Multiple myeloma is the second most common hematologic malignancy following non-Hodgkin's lymphoma, accounting for about 13% of hematological malignant tumors. As an emerging target for multiple myeloma, BCMA antibodies can act on MM cells through a variety of mechanisms.

In the present application, the term “CTLA4” generally refers to the cytotoxic T lymphocyte-associated antigen-4 (also known as CD152), a functional variant thereof and/or a functional fragment thereof. The sequence of CTLA4 is known in the art. For example, the sequence of an exemplary full-length human CTLA4 can be found under Uniprot accession No. P16410; and the sequence of an exemplary full-length cynomolgus monkey CTLA4 can be found under Uniprot accession No. G7PL88. CTLA4 is a negative regulator expressed on T cells. After binding to CD80 or CD86 on antigen presenting cells, it can down-regulate the activity of T cells while blocking the co-stimulatory signal of CD28, thus playing a role in immunosuppression. By blocking the interaction between CTLA4 and its ligand, the activity of T cells can be restored, and the anti-tumor ability can be enhanced. Ipilimumab monoclonal antibody (trade name: Yervoy®) is the first anti-CTLA4 monoclonal antibody drugs approved for marketing. Ipilimumab has a better therapeutic effect on the treatment of advanced melanoma, but also brings about higher immune-related side effects, which seriously affects its clinical application. The toxic and side effects of Ipilimumab are mostly related to the CTLA4 targets, and in the current combination regimens of the PD-1/PD-L1 inhibitor and the CTLA4 inhibitor, the CTLA4 inhibitor, whether Ipilimumab or Tremelimumab, is usually selected at a lower dose. In order to reduce the toxic and side effects of CTLA4 inhibitors, one of the methods worth trying is the targeted delivery of CTLA4 inhibitors into tumor tissues, so that the relevant T cell-mediated responses are limited to the tumor microenvironment, thereby reducing the risk of cytokine release syndrome. For example, antibodies that recognize tumor-associated antigens were used to redirect CTLA4 inhibitors into a specific tumor microenvironment, where they relieve T cell immunosuppressive signals and restore T cell function.

### EXAMPLES

The present invention is further illustrated by the following examples, which are not intended to limit the present invention. The examples do not include detailed descriptions of conventional methods, such as those methods for constructing vectors and plasmids, methods for inserting genes encoding proteins into such vectors and plasmids, or methods for introducing plasmids into host cells. Such methods are well known to those of ordinary skill in the art and are described in numerous publications. Experimental procedures without specified conditions in the following examples are performed in accordance with conventional procedures and conditions, or in accordance with instructions.

### Example 1. Bispecific Binding Proteins Based on Heavy-Chain Antibody

The structures of bispecific binding proteins constructed using heavy-chain antibodies (HCAbs) and their derived single-domain antibodies (sdAbs) involved in the present application are listed in Table 1 and FIG. 1 in this example. Each of the structures is described further below. In the present application, the Fab-HCAb structure is the structure shown as structure (1) or structure (2) in Table 1 and FIG. 1, and is preferably structure (1).

In some structures, the domains were linked via linker peptides. In some structures, amino acid mutations are introduced into the Fc region of the heavy chain to alter its binding to Fc receptors, thereby altering the associated effector functions or other properties. The sequences of the linker peptides that may be used in the structural design of the present application are listed in Table 2.

TABLE 1
Molecular structures of multispecific binding
proteins listed in the present application

| | | | | | Number of |
|---|---|---|---|---|---|
| | | | | | different |
| Structure | Structure | Structure | Binding | | polypeptide |
| No. | mode | type | valence | Symmetry | chains |
| 1 | Fab(CL)-VH-Fc | Fab-HCAb | Tetravalent | Symmetric | 2 |
| 2 | Fab(CH1)-VH-Fc | Fab-HCAb | Tetravalent | Symmetric | 2 |
| 3 | IgG_HC-VH | IgG-VH | Tetravalent | Symmetric | 2 |
| 4 | VH-IgG_HC | VH-IgG | Tetravalent | Symmetric | 2 |
| 5 | FIT-Ig | FIT-Ig | Tetravalent | Symmetric | 3 |

TABLE 2
Sequences of linker peptides

| Name of linker | | | |
|---|---|---|---|
| peptide | Length | Sequence | SEQ ID NO |
| GS_2 | 2 | GS | |
| GS_4 | 4 | GSGS | 161 |
| GS_5 | 5 | GGGGS | 162 |
| GS_7 | 7 | GGGGSGS | 163 |
| GS_15 | 15 | GGGGSGGGGSGGGGS | 164 |
| GS_20 | 20 | GGGGSGGGGSGGGGSGGGGS | 165 |
| GS_25 | 25 | GGGGSGGGGSGGGGSGGGGSGGGGS | 166 |
| H1_15 | 15 | EPKSSDKTHTPPPPP | 167 |
| LH1 | 10 | DKTHTCPPCP | 168 |
| G5-LH | 15 | GGGGGDKTHTCPPCP | 169 |
| H1_15-RT | 17 | EPKSSDKTHTPPPPPRT | 170 |
| L-GS_15-RT | 18 | LGGGGSGGGGSGGGGSRT | 171 |
| L-H1_15-RT | 18 | LEPKSSDKTHTPPPPPRT | 172 |
| KL-H1_15-RT | 19 | KLEPKSSDKTHTPPPPPRT | 173 |
| KL-H1_15-AS | 19 | KLEPKSSDKTHTPPPPPAS | 174 |
| RT-GS_5-KL | 19 | RTGGGGSKL | 175 |
| RT-GS_15-KL | 19 | RTGGGGSGGGGSGGGGSKL | 176 |
| RT-GS_25-KL | 29 | RTGGGGSGGGGSGGGGSGGGGSGGGGS | 177 |
| | | KL | |
| Human IgG1 hinge | 15 | EPKSCDKTHTCPPCP | 178 |
| Human IgG1 hinge | 15 | EPKSSDKTHTCPPCP | 179 |
| (C220S) | | | |
| Human IgG2 hinge | 12 | ERKCCVECPPCP | 180 |
| Human IgG4 hinge | 12 | ESKYGPPCPSCP | 181 |
| Human IgG4 hinge | 12 | ESKYGPPCPPCP | 182 |
| (S228P) | | | |

### Example 1.1 Structures of Bispecific Binding Proteins Containing Heavy-Chain Antibody VH Domains

The present invention provides a method for constructing a bispecific binding protein using two parent monoclonal antibodies: a conventional antibody A (e.g., IgG antibody) binding to a first antigen and a heavy-chain antibody B binding to a second antigen.

As shown in structures (1)-(4) in FIG. 1, the Fab end is derived from a conventional antibody A, wherein VH_A and VL_A are heavy chain and light chain variable regions of a conventional antibody A, respectively. The VH end is derived from a heavy-chain antibody B, wherein VH_B is a heavy chain variable region of the heavy-chain antibody B. CL is a light chain constant region domain. CH1, CH2 and CH3 are first, second and third domains of a heavy chain constant region, respectively. h is a hinge region or a derived sequence of an IgG antibody, and L or L1 or L2 is a linker peptide.

### Example 1.1.1 Structure (1): Fab(CL)-VH-Fc

The binding protein with the structure (1) comprises two different polypeptide chains: a polypeptide chain 1, also known as a short chain, comprising VH_A-CH1 from the amino-terminus to the carboxyl-terminus; and a polypeptide chain 2, also known as a long chain, comprising VL_A-CL-L1-VH_B-L2-CH2-CH3 from the amino-terminus to the carboxyl-terminus. In the structure (1), VL_A of the antibody A and VH_B of the heavy-chain antibody B are fused on the same polypeptide chain, so that the mismatched byproducts generated by the association of VL_A and VH_B can be avoided.

VH_B is linked to CH2 via a linker peptide L2 in the polypeptide chain 2; L2 may be a hinge region or a hinge region-derived linker peptide sequence of IgG or the sequence listed in Table 2, preferably the sequence of human IgG1 hinge region, human IgG1 hinge (C220S) or G5-LH.

In one embodiment, CL is fusion-linked directly to VH_B in the polypeptide chain 2, i.e., L1 is 0 in length. In another embodiment, CL is linked to VH_B via a linker peptide L1 in the polypeptide chain 2; and L1 may be the sequence listed in Table 2.

### Example 1.1.2 Structure (2): Fab(CH1)-VH-Fc

The binding protein with the structure (2) comprises two different polypeptide chains: a polypeptide chain 1, also known as a short chain, comprising VL_A-CL from the amino-terminus to the carboxyl-terminus; and a polypeptide chain 2, also known as a long chain, comprising VH_A-CH1-L1-VH_B-L2-CH2-CH3 from the amino-terminus to the carboxyl-terminus.

VH_B is linked to CH2 via a linker peptide L2 in the polypeptide chain 2; L2 may be a hinge region or a hinge region-derived linker peptide sequence of IgG or the sequence listed in Table 2, preferably the sequence of human IgG1 hinge region, human IgG1 hinge (C220S) or G5-LH.

In one embodiment, CH1 is fusion-linked directly to VH_B in the polypeptide chain 2, i.e., L1 is 0 in length. In another embodiment, CH1 is linked to VH_B via a linker peptide L1 in the polypeptide chain 2; and L1 may be the sequence listed in Table 2.

### Example 1.1.3 Structure (3): IgG_HC-VH

The binding protein with the structure (3) comprises two different polypeptide chains: a polypeptide chain 1, also known as a short chain, comprising VL_A-CL from the amino-terminus to the carboxyl-terminus; and a polypeptide chain 2, also known as a long chain, comprising VH_A-CH1-h-CH2-CH3-L-VH_B from the amino-terminus to the carboxyl-terminus.

In one embodiment, CH3 is fusion-linked directly to VH_B in the polypeptide chain 2, i.e., L is 0 in length. In another embodiment, CH3 is linked to VH_B via a linker peptide L in the polypeptide chain 2; and L may be the sequence listed in Table 2.

### Example 1.1.4 Structure (4): VH-IgG_HC

The binding protein with the structure (4) comprises two different polypeptide chains: a polypeptide chain 1, also known as a short chain, comprising VL_A-CL from the amino-terminus to the carboxyl-terminus; and a polypeptide chain 2, also known as a long chain, comprising VH_B-L-VH_A-CH1-h-CH2-CH3 from the amino-terminus to the carboxyl-terminus.

In one embodiment, VH_B is fusion-linked directly to VH_A in the polypeptide chain 2, i.e., L is 0 in length. In another embodiment, VH_B is linked to VH_A via a linker peptide L in the polypeptide chain 2; and L may be the sequence listed in Table 2.

### Example 1.2 Other Bispecific Binding Protein Structures

### Example 1.2.1 Structure (5): FIT-Ig

The FIT-Ig structure can be designed by referring to WO2015/103072A1, as shown in the structure (5) in FIG. 1. The bispecific antibody molecules with the FIT-Ig structure can be constructed from: a conventional antibody A binding to a first antigen and a conventional antibody B binding to a second antigen.

The binding protein with the structure (5) comprises three polypeptide chains: a polypeptide chain 1 comprising VL_A-CL-L-VH_B-CH1-h-CH2-CH3 from the amino-terminus to the carboxyl-terminus; a polypeptide chain 2 comprising VH_A-CH1 from the amino-terminus to the carboxyl-terminus; and a polypeptide chain 3 comprising VL_B-CL from the amino-terminus to the carboxyl-terminus. VH_A and VL_A are heavy chain and light chain variable regions of a conventional antibody A, respectively; VH_B and VL_B are heavy chain and light chain variable regions of a heavy-chain antibody B, respectively; CL is a domain of a light chain constant region; CH1, CH2 and CH3 are first, second and third domains of a heavy chain constant region, respectively; h is a hinge region or a derived sequence of an IgG antibody; and L is a linker peptide. Generally, the association of the polypeptide chain 2 and the polypeptide chain 3 will result in a mismatched byproduct VH_A-CH1/VL_B-CL.

In one embodiment, CL is fusion-linked directly to VH_B in the polypeptide chain 1, i.e., L is 0 in length. In another embodiment, CL is linked to VH_B via a linker peptide L in the polypeptide chain 1; and L may be the sequence listed in Table 2.

### Example 2. Sequence Analysis, Expression and Purification, and Characterization and Analysis of Physicochemical Properties of Antibodies

### Example 2.1 Expression and Purification of Antibodies

In this example, a general method for preparing antibodies in mammalian host cells (e.g., human embryonic kidney cell HEK293 or Chinese hamster ovary CHO cells and cells derived therefrom) by such techniques as transient transfection and expression, and affinity capture and separation was described. This method is applicable to an antibody of interest comprising Fc. The antibody of interest may consist of one or more protein polypeptide chains, and may be derived from one or more expression plasmids.

The amino acid sequences of the polypeptide chains of the antibody were converted into nucleotide sequences by codon optimization. The encoding nucleotide sequences were synthesized and cloned into expression vectors compatible with the host cell. The mammalian host cells were transfected simultaneously with plasmids encoding the polypeptide chains of the antibody in a particular ratio, and the recombinant antibody with correct folding and assembly of polypeptide chains could be obtained by the conventional recombinant protein expression and purification techniques. Specifically, FreeStyle™ 293-F cells (Thermo, #R79007) were expanded in FreeStyle™ F17 Expression Medium (Thermo, #A1383504). Before transient transfection, the cells were adjusted to a concentration of 6-8×10⁵ cells/mL, and cultured in a shaker at 37° C. with 8% CO₂ for 24 h to make a concentration of 1.2×10⁶ cells/mL. 30 mL of cultured cells were taken. Plasmids encoding the polypeptide chains of the antibody were mixed in a certain ratio, and a total of 30 μg of the plasmids (the ratio of the plasmids to cells was 1 μg:1 mL) were dissolved in 1.5 mL of Opti-MEM reduced serum medium (Thermo, #31985088). The resulting mixture was filtered through a 0.22 μm filter membrane for sterilization. Then, 1.5 mL of Opti-MEM was dissolved in 120 μL of 1 mg/mL PEI (Polysciences, #23966-2), and the mixture was left to stand for 5 min. PEI was slowly added to the plasmids, and the mixture was incubated at room temperature for 10 min. The mixed solution of plasmids and PEI was slowly added dropwise while shaking the culture flask, and the cells were cultured in a shaker at 37° C. with 8% CO₂ for 5 days. Cell viability was measured after 5 days. The culture was collected and centrifuged at 3300 g for 10 min, and then the supernatant was collected and centrifuged at high speed to remove impurities. A gravity column (Bio-Rad, #7311550) containing MabSelect™ (GE Healthcare, #71-5020-91) was equilibrated with a PBS buffer (pH 7.4) and rinsed with 2-5 column volumes of PBS. The column was loaded with the supernatant sample, and rinsed with 5-10 column volumes of PBS buffer, followed by 0.1 M glycine at pH 3.5 to elute the target protein. The eluate was adjusted to neutrality with Tris-HCl at pH 8.0, and concentrated and buffer exchanged into PBS buffer or a buffer with other components with an ultrafiltration tube (Millipore, #UFC901024) to obtain a purified solution of the recombinant antibody. Finally, the purified antibody solution was determined for concentration using NanoDrop (Thermo, NanoDrop™ One), subpackaged and stored for later use.

### Example 2.2 Analysis of Protein Purity and Polymers by SEC-HPLC

In this example, analytical size-exclusion chromatography (SEC) was used to analyze the protein sample for purity and polymer form. An analytical chromatography column TSKgel G3000SWx1 (Tosoh Bioscience, #08541, 5 μm, 7.8 mm×30 cm) was connected to a high-pressure liquid chromatograph HPLC (Agilent Technologies, Agilent 1260 Infinity II) and equilibrated with a PBS buffer at room temperature for at least 1 h. A proper amount of the protein sample (at least 10 μg) was filtered through a 0.22 μm filter membrane and then injected into the system, and an HPLC program was set: the sample was eluted in the chromatography column with a PBS buffer at a flow rate of 1.0 mL/min for a maximum of 25 min. An analysis report was generated by the HPLC, with the retention time of the components with different molecular sizes in the sample reported.

### Example 3. Construction of Bispecific Binding Proteins with Fab-HCAb Structure and Other Structures

In this example, the IgG monoclonal antibodies and HCAb monoclonal antibodies and the derived bispecific binding proteins used in all examples of the present application were summarized.

The information on the IgG monoclonal antibodies and HCAb monoclonal antibodies is listed in Table 3, with the sequence numbers shown in Table 6, and the amino acid sequences shown in Table 11.

The bispecific binding proteins with the Fab-HCAb structure were designed according to the structure described in Example 1.1.1 and FIG. 1 (1) or the structure described in Example 1.1.2 and FIG. 1 (2), and the molecular designs are summarized in Table 4, with the sequence numbers shown in Table 7, and the amino acid sequences shown in Table 12. The protein samples were prepared and analyzed by the method described in Example 2, with the results summarized in Table 9.

The molecular information on the bispecific binding proteins with other structures is summarized in Table 5, with the corresponding structure numbers being structures (3), (4) or (5) in Example land FIG. 1; with the sequence numbers shown in Table 7, and the amino acid sequence is shown in Table 12. The protein samples were prepared and analyzed by the method described in Example 2, with the results summarized in Table 10.

Moreover, the sequence numbers of the corresponding CDR sequences of the protein functional region A (first antigen-binding domain) and the protein functional region B (second antigen-binding domain) of the bispecific binding proteins are listed in Table 8.

In some structures of the binding proteins, amino acid mutations were introduced into the Fc region of the heavy chain to alter its binding to Fc receptors, thereby altering the associated effector functions or other properties. For example, in Table 4 and Table 5, the codes for mutation sites are: AAG: (L234A, L235A, P329G); LALA: (L234A, L235A).

TABLE 3
Control molecules and parent monoclonal antibodies
used in the present application

| Protein | |
|---|---|
| No. | Description |
| PR000628 | Anti-4-1BB monoclonal antibody Urelumab analog (hIgG4) |
| PR003475 | anti-OX40 monoclonal antibody Pogalizumab analog (hIgG1) |
| PR000210 | Anti-HER2 monoclonal antibody Trastuzumab analog (hIgG1) |
| PR000265 | Anti-PD-L1 H2L2 monoclonal antibody 91G3H5H3 (D54E), |
| | hIgG1 (N297A) |
| PR002408 | Anti-B7H4 H2L2 monoclonal antibody 80C8-2E9 (H: G55A; |
| | L: N92Q), hIgG1 |
| PR000197 | Anti-4-1BB H2L2 monoclonal antibody 79B 10G8D4, hIgG4 |
| PR001760 | Anti-4-1BB heavy-chain antibody 1016P0011G10 |
| PR002067 | Anti-OX40 heavy-chain antibody R1026P079E12 |
| PR004433 | Anti-BCMA heavy-chain antibody PR001046_R2_4G10 |
| PR000892 | Anti-BCMA H2L2 monoclonal antibody 1005_21C11E1, |
| | hIgG1 |
| PR000184 | Anti-CTLA4 heavy-chain antibody CL5v3 |

TABLE 4
Bispecific binding proteins with the Fab-HCAb structure in the present application

| | | | | | | First linker | Second linker | |
|---|---|---|---|---|---|---|---|---|
| Structure | Protein | | Fab | | HCAb | peptide (between | peptide (between | Fc type |
| No. | No. | Antigen-1 | antibody A | Antigen-2 | antibody B | Fab and VH_B) | VH_B and CH2) | (mutation) |
| 1 | PR004270 | PD-L1 | PR000265 | 4-1BB | PR001760 | H1_15 | Human IgG1 | Human |
| | | | | | | | hinge | IgG1 |
| | | | | | | | (C220S) | (LALA) |
| 2 | PR007163 | PD-L1 | PR000265 | 4-1BB | PR001760 | None | Human IgG1 | Human |
| | | | | | | | hinge | IgG1 |
| | | | | | | | (C220S) | (LALA) |
| 1 | PR007164 | PD-L1 | PR000265 | 4-1BB | PR001760 | None | Human IgG1 | Human |
| | | | | | | | hinge | IgG1 |
| | | | | | | | (C220S) | (LALA) |
| 1 | PR004279 | B7H4 | PR002408 | 4-1BB | PR001760 | H1_15 | Human IgG1 | Human |
| | | | | | | | hinge | IgG1 |
| | | | | | | | (C220S) | (LALA) |
| 1 | PR004277 | B7H4 | PR002408 | OX40 | PR002067 | H1_15 | Human IgG1 | Human |
| | | | | | | | hinge | IgG1 |
| | | | | | | | (C220S) | (LALA) |
| 1 | PR005744 | BCMA | PR000892 | BCMA | PR004433 | None | Human IgG1 | Human |
| | | | | | | | hinge | IgG1 |
| 1 | PR000305 | HER2 | PR000210 | CTLA4 | PR000184 | None | Human IgG1 | Human |
| | | | | | | | hinge | IgG1 |
| 1 | PR000653 | HER2 | PR000210 | CTLA4 | PR000184 | GS_7 | Human IgG1 | Human |
| | | | | | | | hinge | IgG1 |
| 1 | PR000654 | HER2 | PR000210 | CTLA4 | PR000184 | GS_15 | Human IgG1 | Human |
| | | | | | | | hinge | IgG1 |
| 1 | PR000655 | HER2 | PR000210 | CTLA4 | PR000184 | H1_15 | Human IgG1 | Human |
| | | | | | | | hinge | IgG1 |
| 1 | PR000706 | HER2 | PR000210 | CTLA4 | PR000184 | GS_7 | G5-LH | Human |
| | | | | | | | | IgG1 |

TABLE 5
Bispecific binding proteins with other structures in the present application

| Structure | Protein | | Fab | | HCAb | Description | Linker | Fc type |
|---|---|---|---|---|---|---|---|---|
| No. | No. | Antigen-1 | antibody A | Antigen-2 | antibody B | of structures | peptide | (mutation) |
| 3 | PR003335 | B7H4 | PR002408 | 4-1BB | PR001760 | 4-1BB VH at the | H1_15-RT | Human |
| | | | | | | C-terminus of the | | IgG1 |
| | | | | | | heavy chain of | | (LALA) |
| | | | | | | B7H4 IgG | | |
| 3 | PR003550 | PD-L1 | PR000265 | 4-1BB | PR001760 | 4-1BB VH at the | H1_15-RT | Human |
| | | | | | | C-terminus of the | | IgG1 |
| | | | | | | heavy chain of | | (AAG) |
| | | | | | | PD-L1 IgG | | |
| 3 | PR004276 | B7H4 | PR002408 | OX40 | PR002067 | OX40 VH at the | H1_15-RT | Human |
| | | | | | | C-terminus of the | | IgG1 |
| | | | | | | heavy chain of | | (LALA) |
| | | | | | | B7H4 IgG | | |
| 4 | PR004268 | PD-L1 | PR000265 | 4-1BB | PR001760 | 4-1BB VH at the | GS_15 | Human |
| | | | | | | N-terminus of the | | IgG1 |
| | | | | | | heavy chain of | | (LALA) |
| | | | | | | PD-L1 IgG | | |
| 4 | PR004278 | B7H4 | PR002408 | 4-1BB | PR001760 | 4-1BB VH at the | GS_15 | Human |
| | | | | | | N-terminus of the | | IgG1 |
| | | | | | | heavy chain of | | (LALA) |
| | | | | | | B7H4 IgG | | |
| 5 | PR000701 | PD-L1 | PR000265 | 4-1BB | PR000197 | FIT-Ig; 4-1BB Fab | None | Human |
| | | | | | | close to Fc | | IgG4 |

TABLE 6
Sequence numbers of the variable regions and CDRs of control molecules
and parent monoclonal antibodies in the present application

| Protein | Light | Heavy | | | | | | | | |
|---|---|---|---|---|---|---|---|---|---|---|
| No. | chain | chain | VL | VH | LCDR1 | LCDR2 | LCDR3 | HCDR1 | HCDR2 | HCDR3 |
| PR000628 | 137 | 127 | 119 | 109 | 76 | 86 | 98 | 11 | 33 | 55 |
| PR003475 | 140 | 132 | 122 | 114 | 79 | 88 | 101 | 16 | 38 | 60 |
| PR000210 | 135 | 125 | 117 | 107 | 74 | 84 | 96 | 12 | 31 | 53 |
| PR000265 | 136 | 126 | 118 | 108 | 75 | 85 | 97 | 13 | 32 | 54 |
| PR002408 | 139 | 131 | 121 | 113 | 78 | 83 | 100 | 15 | 37 | 59 |
| PR000197 | 134 | 124 | 116 | 106 | 73 | 83 | 95 | 11 | 30 | 52 |
| PR001760 | | 129 | | 111 | | | | 14 | 35 | 57 |
| PR002067 | | 130 | | 112 | | | | 13 | 36 | 58 |
| PR004433 | | 133 | | 115 | | | | 17 | 39 | 61 |
| PR000892 | 138 | 128 | 120 | 110 | 77 | 87 | 99 | 13 | 34 | 56 |
| PR000184 | | 123 | | 105 | | | | 10 | 29 | 51 |

TABLE 7
Sequence numbers of bispecific binding
proteins in the present application

| Structure | Protein | Polypeptide | Polypeptide | Polypeptide |
|---|---|---|---|---|
| No. | No. | chain 1 | chain 2 | chain 3 |
| 1 | PR004270 | 147 | 153 | |
| 2 | PR007163 | 136 | 183 | |
| 1 | PR007164 | 147 | 184 | |
| 1 | PR004279 | 155 | 158 | |
| 1 | PR004277 | 155 | 156 | |
| 1 | PR005744 | 159 | 160 | |
| 1 | PR000305 | 141 | 142 | |
| 1 | PR000653 | 141 | 143 | |
| 1 | PR000654 | 141 | 144 | |
| 1 | PR000655 | 141 | 145 | |
| 1 | PR000706 | 141 | 149 | |
| 3 | PR003335 | 139 | 150 | |
| 3 | PR003550 | 136 | 151 | |
| 3 | PR004276 | 139 | 154 | |
| 4 | PR004268 | 136 | 152 | |
| 4 | PR004278 | 139 | 157 | |
| 5 | PR000701 | 148 | 147 | 146 |

TABLE 8
Sequence numbers of CDRs of the antigen-binding domains
of bispecific binding proteins in the present application

| | | Protein | | | | | | |
|---|---|---|---|---|---|---|---|---|
| Structure | Protein | functional | | | | | | |
| No. | No. | region | LCDR1 | LCDR2 | LCDR3 | HCDR1 | HCDR2 | HCDR3 |
| 1 | PR004270, | A | 75 | 85 | 97 | 13 | 32 | 54 |
| | PR007164 | B | | | | 14 | 35 | 57 |
| 1 | PR004279 | A | 78 | 83 | 100 | 15 | 37 | 59 |
| | | B | | | | 14 | 35 | 57 |
| 1 | PR004277 | A | 78 | 83 | 100 | 15 | 37 | 59 |
| | | B | | | | 13 | 36 | 58 |
| 1 | PR005744 | A | 77 | 87 | 99 | 13 | 34 | 56 |
| | | B | | | | 17 | 39 | 61 |
| 1 | PR000305, | A | 74 | 84 | 96 | 12 | 31 | 53 |
| | PR000653, | B | | | | 10 | 29 | 51 |
| | PR000654, | | | | | | | |
| | PR000655, | | | | | | | |
| | PR000706 | | | | | | | |
| 2 | PR007163 | A | 75 | 85 | 97 | 13 | 32 | 54 |
| | | B | | | | 14 | 35 | 57 |
| 3 | PR003335 | A | 78 | 83 | 100 | 15 | 37 | 59 |
| | | B | | | | 14 | 35 | 57 |
| 3 | PR003550 | A | 75 | 85 | 97 | 13 | 32 | 54 |
| | | B | | | | 14 | 35 | 57 |
| 3 | PR004276 | A | 78 | 83 | 100 | 15 | 37 | 59 |
| | | B | | | | 13 | 36 | 58 |
| 4 | PR004268 | A | 75 | 85 | 97 | 13 | 32 | 54 |
| | | B | | | | 14 | 35 | 57 |
| 4 | PR004278 | A | 78 | 83 | 100 | 15 | 37 | 59 |
| | | B | | | | 14 | 35 | 57 |
| 5 | PR000701 | A | 75 | 85 | 97 | 13 | 32 | 54 |
| | | B | 73 | 83 | 95 | 11 | 30 | 52 |

TABLE 9
Expression of bispecific binding proteins with the
Fab-HCAb structure in the present application

| | | | Plasmid | | |
|---|---|---|---|---|---|
| | | | transfection | | |
| | | Expression | ratio (short | Yield (mg/L) | SEC-HPLC |
| Structure | Protein | system and | chain:long | after first | purity |
| No. | No. | volume | chain) | purification | (%) |
| 1 | PR004270 | HEK293-6E (40 ml) | 3:2 | 77.5 | 92.33 |
| 2 | PR007163 | HEK293 (100 ml) | 3:1 | 13 | 97.84 |
| 1 | PR007164 | HEK293 (100 ml) | 3:1 | 47 | 93.37 |
| 1 | PR004279 | HEK293-F (30 ml) | 3:2 | 11.4 | 79.18 |
| 1 | PR004279 | CHO (100 ml) | 3:2 | 3.6 | 95.46 |
| 1 | PR004277 | CHO (100 ml) | 3:2 | 48 | 93.03 |
| 1 | PR005744 | HEK293-F (100 ml) | 3:2 | 51.9 | 99.34 |
| 1 | PR000305 | HEK293-F (30 ml) | 3:2 | 70.0 | 87.17 |
| 1 | PR000653 | HEK293-F (30 ml) | 3:2 | 69.9 | 100.00 |
| 1 | PR000654 | HEK293-F (30 ml) | 3:2 | 62.9 | 100.00 |
| 1 | PR000655 | HEK293-F (30 ml) | 3:2 | 102.1 | 97.82 |
| 1 | PR000706 | HEK293-F (30 ml) | 3:2 | 40.9 | 100.00 |

TABLE 10
Expression of bispecific binding proteins with
other structures in the present application

| | | Expression | Yield (mg/L) | HPLC-SEC |
|---|---|---|---|---|
| Structure | Protein | system and | after first | purity |
| No. | No. | volume | purification | (%) |
| 3 | PR003335 | ExpiCHO (200 ml) | 68.9 | 99.29 |
| 3 | PR003550 | HEK293-F (30 ml) | 42.2 | 95.41 |
| 3 | PR004276 | HEK293-6E (40 ml) | 17.7 | 95.46 |
| 4 | PR004268 | HEK293-6E (40 ml) | 31.5 | 97.94 |
| 4 | PR004278 | HEK293-6E (40 ml) | 11.5 | 98.08 |
| 5 | PR000701 | HEK293-F (30 ml) | 198.0 | 79.88 |

TABLE 11
Amino acid sequences of control molecules and parent monoclonal
antibodies in the present application

| Protein | SEQ ID | | |
|---|---|---|---|
| No. | NO | Fragment | Amino acid sequence |
| PR000628 | 137 | Light chain | EIVLTQSPATLSLSPGERATLSCRASQSVSSYLAWYQQK |
| | | | PGQAPRLLIYDASNRATGIPARFSGSGSGTDFTLTISSLE |
| | | | PEDFAVYYCQQRSNWPPALTFGGGTKVEIKRTVAAPSV |
| | | | FIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 119 | VL | EIVLTQSPATLSLSPGERATLSCRASQSVSSYLAWYQQK |
| | | | PGQAPRLLIYDASNRATGIPARFSGSGSGTDFTLTISSLE |
| | | | PEDFAVYYCQQRSNWPPALTFGGGTKVEIK |
| | 76 | LCDR1 | RASQSVSSYLA |
| | 86 | LCDR2 | DASNRAT |
| | 98 | LCDR3 | QQRSNWPPALT |
| | 127 | Heavy | QVQLQQWGAGLLKPSETLSLTCAVYGGSFSGYYWSWI |
| | | chain | RQSPEKGLEWIGEINHGGYVTYNPSLESRVTISVDTSK |
| | | | NQFSLKLSSVTAADTAVYYCARDYGPGNYDWYFDLW |
| | | | GRGTLVTVSSASTKGPSVFPLAPCSRSTSESTAALGCLV |
| | | | KDYFPEPVTVSWNSGALTSGVHTFPAVLQSSGLYSLSS |
| | | | VVTVPSSSLGTKTYTCNVDHKPSNTKVDKRVESKYGP |
| | | | PCPPCPAPEFLGGPSVFLFPPKPKDTLMISRTPEVTCVV |
| | | | VDVSQEDPEVQFNWYVDGVEVHNAKTKPREEQFNST |
| | | | YRVVSVLTVLHQDWLNGKEYKCKVSNKGLPSSIEKTI |
| | | | SKAKGQPREPQVYTLPPSQEEMTKNQVSLTCLVKGFY |
| | | | PSDIAVEWESNGQPENNYKTTPPVLDSDGSFFLYSRLT |
| | | | VDKSRWQEGNVFSCSVMHEALHNHYTQKSLSLSLGK |
| | 109 | VH | QVQLQQWGAGLLKPSETLSLTCAVYGGSFSGYYWSWI |
| | | | RQSPEKGLEWIGEINHGGYVTYNPSLESRVTISVDTSK |
| | | | NQFSLKLSSVTAADTAVYYCARDYGPGNYDWYFDLW |
| | | | GRGTLVTVSS |
| | 11 | HCDR1 | GGSFSGY |
| | 33 | HCDR2 | NHGGY |
| | 55 | HCDR3 | DYGPGNYDWYFDL |
| PR003475 | 140 | Light chain | DIQMTQSPSSLSASVGDRVTITCRASQDISNYLNWYQQ |
| | | | KPGKAPKLLIYYTSRLRSGVPSRFSGSGSGTDFTLTISS |
| | | | LQPEDFATYYCQQGHTLPPTFGQGTKVEIKRTVAAPSV |
| | | | FIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 122 | VL | DIQMTQSPSSLSASVGDRVTITCRASQDISNYLNWYQQ |
| | | | KPGKAPKLLIYYTSRLRSGVPSRFSGSGSGTDFTLTISS |
| | | | LQPEDFATYYCQQGHTLPPTFGQGTKVEIK |
| | 79 | LCDR1 | RASQDISNYLN |
| | 88 | LCDR2 | YTSRLRS |
| | 101 | LCDR3 | QQGHTLPPT |
| | 132 | Heavy | EVQLVQSGAEVKKPGASVKVSCKASGYTFTDSYMSW |
| | | chain | VRQAPGQGLEWIGDMYPDNGDSSYNQKFRERVTITRD |
| | | | TSTSTAYLELSSLRSEDTAVYYCVLAPRWYFSVWGQG |
| | | | TLVTVSSASTKGPSVFPLAPSSKSTSGGTAALGCLVKD |
| | | | YFPEPVTVSWNSGALTSGVHTFPAVLQSSGLYSLSSVV |
| | | | TVPSSSLGTQTYICNVNHKPSNTKVDKKVEPKSCDKT |
| | | | HTCPPCPAPELLGGPSVFLFPPKPKDTLMISRTPEVTCV |
| | | | VVDVSHEDPEVKFNWYVDGVEVHNAKTKPREEQYNS |
| | | | TYRVVSVLTVLHQDWLNGKEYKCKVSNKALPAPIEKT |
| | | | ISKAKGQPREPQVYTLPPSREEMTKNQVSLTCLVKGFY |
| | | | PSDIAVEWESNGQPENNYKTTPPVLDSDGSFFLYSKLT |
| | | | VDKSRWQQGNVFSCSVMHEALHNHYTQKSLSLSPGK |
| | 114 | VH | EVQLVQSGAEVKKPGASVKVSCKASGYTFTDSYMSW |
| | | | VRQAPGQGLEWIGDMYPDNGDSSYNQKFRERVTITRD |
| | | | TSTSTAYLELSSLRSEDTAVYYCVLAPRWYFSVWGQG |
| | | | TLVTVSS |
| | 16 | HCDR1 | GYTFTDS |
| | 38 | HCDR2 | YPDNGD |
| | 60 | HCDR3 | APRWYFSV |
| PR000210 | 135 | Light chain | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVF |
| | | | IFPPSDEQLKSGTASVVCLLNNFYPREAKVQWKVDNA |
| | | | LQSGNSQESVTEQDSKDSTYSLSSTLTLSKADYEKHKV |
| | | | YACEVTHQGLSSPVTKSFNRGEC |
| | 117 | VL | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIK |
| | 74 | LCDR1 | RASQDVNTAVA |
| | 84 | LCDR2 | SASFLYS |
| | 96 | LCDR3 | QQHYTTPPT |
| | 125 | Heavy | EVQLVESGGGLVQPGGSLRLSCAASGFNIKDTYIHWV |
| | | chain | RQAPGKGLEWVARIYPTNGYTRYADSVKGRFTISADT |
| | | | SKNTAYLQMNSLRAEDTAVYYCSRWGGDGFYAMDY |
| | | | WGQGTLVTVSSASTKGPSVFPLAPSSKSTSGGTAALGC |
| | | | LVKDYFPEPVTVSWNSGALTSGVHTFPAVLQSSGLYSL |
| | | | SSVVTVPSSSLGTQTYICNVNHKPSNTKVDKKVEPKSC |
| | | | DKTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPI |
| | | | EKTISKAKGQPREPQVYTLPPSREEMTKNQVSLTCLVK |
| | | | GFYPSDIAVEWESNGQPENNYKTTPPVLDSDGSFFLYS |
| | | | KLTVDKSRWQQGNVFSCSVMHEALHNHYTQKSLSLS |
| | | | PGK |
| | 107 | VH | EVQLVESGGGLVQPGGSLRLSCAASGFNIKDTYIHWV |
| | | | RQAPGKGLEWVARIYPTNGYTRYADSVKGRFTISADT |
| | | | SKNTAYLQMNSLRAEDTAVYYCSRWGGDGFYAMDY |
| | | | WGQGTLVTVSS |
| | 12 | HCDR1 | GFNIKDT |
| | 31 | HCDR2 | YPTNGY |
| | 53 | HCDR3 | WGGDGFYAMDY |
| PR000265 | 136 | Light chain | DIQMTQSPSTLSASVGDRVTVTCRASQSIYIWLAWYQ |
| | | | QKPGKAPNLLIYKASSLETGVPSRFSGSGSGTEFTLTIS |
| | | | SLQPDDFATYYCQQYYGSSRTFGQGTKVEIKRTVAAPS |
| | | | VFIFPPSDEQLKSGTASVVCLLNNFYPREAKVQWKVD |
| | | | NALQSGNSQESVTEQDSKDSTYSLSSTLTLSKADYEKH |
| | | | KVYACEVTHQGLSSPVTKSFNRGEC |
| | 118 | VL | DIQMTQSPSTLSASVGDRVTVTCRASQSIYIWLAWYQ |
| | | | QKPGKAPNLLIYKASSLETGVPSRFSGSGSGTEFTLTIS |
| | | | SLQPDDFATYYCQQYYGSSRTFGQGTKVEIK |
| | 75 | LCDR1 | RASQSIYIWLA |
| | 85 | LCDR2 | KASSLET |
| | 97 | LCDR3 | QQYYGSSRT |
| | 126 | Heavy | EVQLVESGGGLVQPGGSLRLSCAASGFTFSSYWMSWV |
| | | chain | RQAPGKGLEWVANIKQEGSEKYYVDSVKGRFTISRDN |
| | | | AKNSLYLQMNSLRAEDTAVYYCARDRAVAGAFDIWG |
| | | | QGTMVTVSSASTKGPSVFPLAPSSKSTSGGTAALGCLV |
| | | | KDYFPEPVTVSWNSGALTSGVHTFPAVLQSSGLYSLSS |
| | | | VVTVPSSSLGTQTYICNVNHKPSNTKVDKKVEPKSCD |
| | | | KTHTCPPCPAPELLGGPSVFLFPPKPKDTLMISRTPEVT |
| | | | CVVVDVSHEDPEVKFNWYVDGVEVHNAKTKPREEQY |
| | | | ASTYRVVSVLTVLHQDWLNGKEYKCKVSNKALPAPIE |
| | | | KTISKAKGQPREPQVYTLPPSREEMTKNQVSLTCLVKG |
| | | | FYPSDIAVEWESNGQPENNYKTTPPVLDSDGSFFLYSK |
| | | | LTVDKSRWQQGNVFSCSVMHEALHNHYTQKSLSLSP |
| | | | GK |
| | 108 | VH | EVQLVESGGGLVQPGGSLRLSCAASGFTFSSYWMSWV |
| | | | RQAPGKGLEWVANIKQEGSEKYYVDSVKGRFTISRDN |
| | | | AKNSLYLQMNSLRAEDTAVYYCARDRAVAGAFDIWG |
| | | | QGTMVTVSS |
| | 13 | HCDR1 | GFTFSSY |
| | 32 | HCDR2 | KQEGSE |
| | 54 | HCDR3 | DRAVAGAFDI |
| PR002408 | 139 | Light chain | EIVMTQSPATLSVSPGERATLSCRASQSISSNLGWYQQ |
| | | | KPGQAPRLLIYGASTRATGIPARFSGSGSGTEFTLTISSL |
| | | | QSEDFAVYYCQQYQSWPPLTFGGGTKVEIKRTVAAPS |
| | | | VFIFPPSDEQLKSGTASVVCLLNNFYPREAKVQWKVD |
| | | | NALQSGNSQESVTEQDSKDSTYSLSSTLTLSKADYEKH |
| | | | KVYACEVTHQGLSSPVTKSFNRGEC |
| | 121 | VL | EIVMTQSPATLSVSPGERATLSCRASQSISSNLGWYQQ |
| | | | KPGQAPRLLIYGASTRATGIPARFSGSGSGTEFTLTISSL |
| | | | QSEDFAVYYCQQYQSWPPLTFGGGTKVEIK |
| | 78 | LCDR1 | RASQSISSNLG |
| | 83 | LCDR2 | GASTRAT |
| | 100 | LCDR3 | QQYQSWPPLT |
| | 131 | Heavy | QVQLVESGGGVVQPGRSLRLSCAASGFTFRSFGMHW |
| | | chain | VRQAPGKGLEWVAVISYDASNEYYADSVKGRFIISRDN |
| | | | SKDTLYLQMNSLRAGDTAVYYCAKGGGLRWYFAYW |
| | | | GQGTLVTVSSASTKGPSVFPLAPSSKSTSGGTAALGCL |
| | | | VKDYFPEPVTVSWNSGALTSGVHTFPAVLQSSGLYSLS |
| | | | SVVTVPSSSLGTQTYICNVNHKPSNTKVDKKVEPKSC |
| | | | DKTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPI |
| | | | EKTISKAKGQPREPQVYTLPPSREEMTKNQVSLTCLVK |
| | | | GFYPSDIAVEWESNGQPENNYKTTPPVLDSDGSFFLYS |
| | | | KLTVDKSRWQQGNVFSCSVMHEALHNHYTQKSLSLS |
| | | | PGK |
| | 113 | VH | QVQLVESGGGVVQPGRSLRLSCAASGFTFRSFGMHW |
| | | | VRQAPGKGLEWVAVISYDASNEYYADSVKGRFIISRDN |
| | | | SKDTLYLQMNSLRAGDTAVYYCAKGGGLRWYFAYW |
| | | | GQGTLVTVSS |
| | 15 | HCDR1 | GFTFRSF |
| | 37 | HCDR2 | SYDASN |
| | 59 | HCDR3 | GGGLRWYFAY |
| PR000197 | 134 | Light chain | EFVMTQSPATLSVSPGERATLSCRASQSISSILAWYQQK |
| | | | PGQAPRLLIYGASTRATGIPARFSGSGSGTEFTLTISSLQ |
| | | | SEDFAVYYCQQYYNWPLTFGGGTKVEIKRTVAAPSVFI |
| | | | FPPSDEQLKSGTASVVCLLNNFYPREAKVQWKVDNAL |
| | | | QSGNSQESVTEQDSKDSTYSLSSTLTLSKADYEKHKVY |
| | | | ACEVTHQGLSSPVTKSFNRGEC |
| | 116 | VL | EFVMTQSPATLSVSPGERATLSCRASQSISSILAWYQQK |
| | | | PGQAPRLLIYGASTRATGIPARFSGSGSGTEFTLTISSLQ |
| | | | SEDFAVYYCQQYYNWPLTFGGGTKVEIK |
| | 73 | LCDR1 | RASQSISSILA |
| | 83 | LCDR2 | GASTRAT |
| | 95 | LCDR3 | QQYYNWPLT |
| | 124 | Heavy | QVQLQQWGAGLLKPSETLSLTCAVYGGSFSGYYWSWI |
| | | chain | RQPPGKGLEWIGEINHSGSTDSNPSLKGRVTFSVDTSK |
| | | | NQFSLKLNSVTAADTAVYYCARLTGPFDYWGQGTLVT |
| | | | VSSASTKGPSVFPLAPCSRSTSESTAALGCLVKDYFPEP |
| | | | VTVSWNSGALTSGVHTFPAVLQSSGLYSLSSVVTVPSS |
| | | | SLGTKTYTCNVDHKPSNTKVDKRVESKYGPPCPPCPA |
| | | | PEFLGGPSVFLFPPKPKDTLMISRTPEVTCVVVDVSQE |
| | | | DPEVQFNWYVDGVEVHNAKTKPREEQFNSTYRVVSV |
| | | | LTVLHQDWLNGKEYKCKVSNKGLPSSIEKTISKAKGQ |
| | | | PREPQVYTLPPSQEEMTKNQVSLTCLVKGFYPSDIAVE |
| | | | WESNGQPENNYKTTPPVLDSDGSFFLYSRLTVDKSRW |
| | | | QEGNVFSCSVMHEALHNHYTQKSLSLSLGK |
| | 106 | VH | QVQLQQWGAGLLKPSETLSLTCAVYGGSFSGYYWSWI |
| | | | RQPPGKGLEWIGEINHSGSTDSNPSLKGRVTFSVDTSK |
| | | | NQFSLKLNSVTAADTAVYYCARLTGPFDYWGQGTLVT |
| | | | VSS |
| | 11 | HCDR1 | GGSFSGY |
| | 30 | HCDR2 | NHSGS |
| | 52 | HCDR3 | LTGPFDY |
| PR001760 | 129 | Heavy | EVQLVESGGGVVQPGGSLRLSCAASGFTFSNYAMTW |
| | | chain | VRQAPEKGLEWVSSISGSGVSTYYADSVKGRFTISRDN |
| | | | SKNTLYLQMTRLTAEDTAVYFCAKEGSSETDDHYYNV |
| | | | DVWGQGTTVTVSSEPKSCDKTHTCPPCPAPELLGGPS |
| | | | VFLFPPKPKDTLMISRTPEVTCVVVDVSHEDPEVKFN |
| | | | WYVDGVEVHNAKTKPREEQYNSTYRVVSVLTVLHQD |
| | | | WLNGKEYKCKVSNKALPAPIEKTISKAKGQPREPQVY |
| | | | TLPPSREEMTKNQVSLTCLVKGFYPSDIAVEWESNGQP |
| | | | ENNYKTTPPVLDSDGSFFLYSKLTVDKSRWQQGNVFS |
| | | | CSVMHEALHNHYTQKSLSLSPGK |
| | 111 | VH | EVQLVESGGGVVQPGGSLRLSCAASGFTFSNYAMTW |
| | | | VRQAPEKGLEWVSSISGSGVSTYYADSVKGRFTISRDN |
| | | | SKNTLYLQMTRLTAEDTAVYFCAKEGSSETDDHYYNV |
| | | | DVWGQGTTVTVSS |
| | 14 | HCDR1 | GFTFSNY |
| | 35 | HCDR2 | SGSGVS |
| | 57 | HCDR3 | EGSSETDDHYYNVDV |
| PR002067 | 130 | Heavy | EVQLLESGGGLVQPGGSLRLSCAASGFTFSSYAMSWV |
| | | chain | RQAPGKGLEWVSAISGRGGSTFYADSVRGRFTISRDNS |
| | | | KNTLYLQMNSLRAEDTAMYYCVDGTTGTTDVDYWG |
| | | | QGTLVTVSSEPKSCDKTHTCPPCPAPELLGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNGK |
| | | | EYKCKVSNKALPAPIEKTISKAKGQPREPQVYTLPPSR |
| | | | EEMTKNQVSLTCLVKGFYPSDIAVEWESNGQPENNYK |
| | | | TTPPVLDSDGSFFLYSKLTVDKSRWQQGNVFSCSVMH |
| | | | EALHNHYTQKSLSLSPGK |
| | 112 | VH | EVQLLESGGGLVQPGGSLRLSCAASGFTFSSYAMSWV |
| | | | RQAPGKGLEWVSAISGRGGSTFYADSVRGRFTISRDNS |
| | | | KNTLYLQMNSLRAEDTAMYYCVDGTTGTTDVDYWG |
| | | | QGTLVTVSS |
| | 13 | HCDR1 | GFTFSSY |
| | 36 | HCDR2 | SGRGGS |
| | 58 | HCDR3 | GTTGTTDVDY |
| PR004433 | 133 | Heavy | EVQLVETGGGLIQPGGSLRLSCAASGFTVSDSYMTWV |
| | | chain | RQAPGKGLEWVSVIFSGGRTYYSDSVKGRFTISRDNA |
| | | | KNTLYLQMNSLRAEDTALYYCARRNYDDTRGTDVFDI |
| | | | WGQGTMVTVSSEPKSCDKTHTCPPCPAPELLGGPSVF |
| | | | LFPPKPKDTLMISRTPEVTCVVVDVSHEDPEVKFNWY |
| | | | VDGVEVHNAKTKPREEQYNSTYRVVSVLTVLHQDWL |
| | | | NGKEYKCKVSNKALPAPIEKTISKAKGQPREPQVYTLP |
| | | | PSREEMTKNQVSLTCLVKGFYPSDIAVEWESNGQPEN |
| | | | NYKTTPPVLDSDGSFFLYSKLTVDKSRWQQGNVFSCS |
| | | | VMHEALHNHYTQKSLSLSPGK |
| | 115 | VH | EVQLVETGGGLIQPGGSLRLSCAASGFTVSDSYMTWV |
| | | | RQAPGKGLEWVSVIFSGGRTYYSDSVKGRFTISRDNA |
| | | | KNTLYLQMNSLRAEDTALYYCARRNYDDTRGTDVFDI |
| | | | WGQGTMVTVSS |
| | 17 | HCDR1 | GFTVSDS |
| | 39 | HCDR2 | FSGGR |
| | 61 | HCDR3 | RNYDDTRGTDVFDI |
| PR000892 | 138 | Light chain | DIQMTQSPSTLSASVGDRVTITCRASQSISSWLAWYQQ |
| | | | KPGKAPKLLIYKASSLESGVPSRFSGSGSGTEFTLTISSL |
| | | | QTDDFATYYCQQYNSYLFTFGQGTKLEIKRTVAAPSVF |
| | | | IFPPSDEQLKSGTASVVCLLNNFYPREAKVQWKVDNA |
| | | | LQSGNSQESVTEQDSKDSTYSLSSTLTLSKADYEKHKV |
| | | | YACEVTHQGLSSPVTKSFNRGEC |
| | 120 | VL | DIQMTQSPSTLSASVGDRVTITCRASQSISSWLAWYQQ |
| | | | KPGKAPKLLIYKASSLESGVPSRFSGSGSGTEFTLTISSL |
| | | | QTDDFATYYCQQYNSYLFTFGQGTKLEIK |
| | 77 | LCDR1 | RASQSISSWLA |
| | 87 | LCDR2 | KASSLES |
| | 99 | LCDR3 | QQYNSYLFT |
| | 128 | Heavy | QVQLVESGGGVVQPGRSLRLSCAATGFTFSSYGMYW |
| | | chain | VRQAPGKGLEWVAAIWNDGSNNYYADSVKGRFTISR |
| | | | DDSKNTLNLQMNSLRAEDTAMYYCARDRLPMASLRY |
| | | | FDWLGVMDAWGQGTSVTVSSASTKGPSVFPLAPSSKS |
| | | | TSGGTAALGCLVKDYFPEPVTVSWNSGALTSGVHTFPA |
| | | | VLQSSGLYSLSSVVTVPSSSLGTQTYICNVNHKPSNTK |
| | | | VDKKVEPKSCDKTHTCPPCPAPELLGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEYK |
| | | | CKVSNKALPAPIEKTISKAKGQPREPQVYTLPPSREEM |
| | | | TKNQVSLTCLVKGFYPSDIAVEWESNGQPENNYKTTPP |
| | | | VLDSDGSFFLYSKLTVDKSRWQQGNVFSCSVMHEALH |
| | | | NHYTQKSLSLSPGK |
| | 110 | VH | QVQLVESGGGVVQPGRSLRLSCAATGFTFSSYGMYW |
| | | | VRQAPGKGLEWVAAIWNDGSNNYYADSVKGRFTISR |
| | | | DDSKNTLNLQMNSLRAEDTAMYYCARDRLPMASLRY |
| | | | FDWLGVMDAWGQGTSVTVSS |
| | 13 | HCDR1 | GFTFSSY |
| | 34 | HCDR2 | WNDGSN |
| | 56 | HCDR3 | DRLPMASLRYFDWLGVMDA |
| PR000184 | 123 | Heavy | EVQLVESGGGLIQPGGSLRLSCAVSGFTVSKNYMSWV |
| | | chain | RQAPGKGLEWVSVVYSGGSKTYADSVKGRFTISRDNS |
| | | | KNTLYLQMNSLRAEDTAVYYCARAVPHSPSSFDIWGQ |
| | | | GTMVTVSSEPKSCDKTHTCPPCPAPELLGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNGK |
| | | | EYKCKVSNKALPAPIEKTISKAKGQPREPQVYTLPPSR |
| | | | EEMTKNQVSLTCLVKGFYPSDIAVEWESNGQPENNYK |
| | | | TTPPVLDSDGSFFLYSKLTVDKSRWQQGNVFSCSVMH |
| | | | EALHNHYTQKSLSLSPGK |
| | 105 | VH | EVQLVESGGGLIQPGGSLRLSCAVSGFTVSKNYMSWV |
| | | | RQAPGKGLEWVSVVYSGGSKTYADSVKGRFTISRDNS |
| | | | KNTLYLQMNSLRAEDTAVYYCARAVPHSPSSFDIWGQ |
| | | | GTMVTVSS |
| | 10 | HCDR1 | GFTVSKN |
| | 29 | HCDR2 | YSGGS |
| | 51 | HCDR3 | AVPHSPSSFDI |

TABLE 12
Amino acid sequences of bispecific binding proteins in the present
application

| Protein | SEQ ID | | |
|---|---|---|---|
| No. | NO | Fragment | Amino acid sequence |
| PR004270 | 147 | Short chain | EVQLVESGGGLVQPGGSLRLSCAASGFTFSSYWMSWVR |
| | | | QAPGKGLEWVANIKQEGSEKYYVDSVKGRFTISRDNAK |
| | | | NSLYLQMNSLRAEDTAVYYCARDRAVAGAFDIWGQGT |
| | | | MVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSC |
| | 153 | Long chain | DIQMTQSPSTLSASVGDRVTVTCRASQSIYIWLAWYQQK |
| | | | PGKAPNLLIYKASSLETGVPSRFSGSGSGTEFTLTISSLQP |
| | | | DDFATYYCQQYYGSSRTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEPKSSDKTHTPPPPPEVQLVES |
| | | | GGGVVQPGGSLRLSCAASGFTFSNYAMTWVRQAPEKG |
| | | | LEWVSSISGSGVSTYYADSVKGRFTISRDNSKNTLYLQM |
| | | | TRLTAEDTAVYFCAKEGSSETDDHYYNVDVWGQGTTVT |
| | | | VSSEPKSSDKTHTCPPCPAPEAAGGPSVFLFPPKPKDTLM |
| | | | ISRTPEVTCVVVDVSHEDPEVKFNWYVDGVEVHNAKTK |
| | | | PREEQYNSTYRVVSVLTVLHQDWLNGKEYKCKVSNKA |
| | | | LPAPIEKTISKAKGQPREPQVYTLPPSREEMTKNQVSLTC |
| | | | LVKGFYPSDIAVEWESNGQPENNYKTTPPVLDSDGSFFL |
| | | | YSKLTVDKSRWQQGNVFSCSVMHEALHNHYTQKSLSLS |
| | | | PGK |
| PR007163 | 136 | Short chain | DIQMTQSPSTLSASVGDRVTVTCRASQSIYIWLAWYQQK |
| | | | PGKAPNLLIYKASSLETGVPSRFSGSGSGTEFTLTISSLQP |
| | | | DDFATYYCQQYYGSSRTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |
| | 183 | Long chain | EVQLVESGGGLVQPGGSLRLSCAASGFTFSSYWMSWVR |
| | | | QAPGKGLEWVANIKQEGSEKYYVDSVKGRFTISRDNAK |
| | | | NSLYLQMNSLRAEDTAVYYCARDRAVAGAFDIWGQGT |
| | | | MVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCEVQLVESG |
| | | | GGVVQPGGSLRLSCAASGFTFSNYAMTWVRQAPEKGLE |
| | | | WVSSISGSGVSTYYADSVKGRFTISRDNSKNTLYLQMTR |
| | | | LTAEDTAVYFCAKEGSSETDDHYYNVDVWGQGTTVTVS |
| | | | SEPKSSDKTHTCPPCPAPEAAGGPSVFLFPPKPKDTLMIS |
| | | | RTPEVTCVVVDVSHEDPEVKFNWYVDGVEVHNAKTKP |
| | | | REEQYNSTYRVVSVLTVLHQDWLNGKEYKCKVSNKAL |
| | | | PAPIEKTISKAKGQPREPQVYTLPPSREEMTKNQVSLTCL |
| | | | VKGFYPSDIAVEWESNGQPENNYKTTPPVLDSDGSFFLY |
| | | | SKLTVDKSRWQQGNVFSCSVMHEALHNHYTQKSLSLSP |
| | | | GK |
| PR007164 | 147 | Short chain | EVQLVESGGGLVQPGGSLRLSCAASGFTFSSYWMSWVR |
| | | | QAPGKGLEWVANIKQEGSEKYYVDSVKGRFTISRDNAK |
| | | | NSLYLQMNSLRAEDTAVYYCARDRAVAGAFDIWGQGT |
| | | | MVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSC |
| | 184 | Long chain | DIQMTQSPSTLSASVGDRVTVTCRASQSIYIWLAWYQQK |
| | | | PGKAPNLLIYKASSLETGVPSRFSGSGSGTEFTLTISSLQP |
| | | | DDFATYYCQQYYGSSRTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEVQLVESGGGVVQPGGSLRLS |
| | | | CAASGFTFSNYAMTWVRQAPEKGLEWVSSISGSGVSTY |
| | | | YADSVKGRFTISRDNSKNTLYLQMTRLTAEDTAVYFCAK |
| | | | EGSSETDDHYYNVDVWGQGTTVTVSSEPKSSDKTHTCP |
| | | | PCPAPEAAGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| PR004279 | 155 | Short chain | QVQLVESGGGVVQPGRSLRLSCAASGFTFRSFGMHWVR |
| | | | QAPGKGLEWVAVISYDASNEYYADSVKGRFIISRDNSKD |
| | | | TLYLQMNSLRAGDTAVYYCAKGGGLRWYFAYWGQGTL |
| | | | VTVSSASTKGPSVFPLAPSSKSTSGGTAALGCLVKDYFPE |
| | | | PVTVSWNSGALTSGVHTFPAVLQSSGLYSLSSVVTVPSSS |
| | | | LGTQTYICNVNHKPSNTKVDKKVEPKSC |
| | 158 | Long chain | EIVMTQSPATLSVSPGERATLSCRASQSISSNLGWYQQKP |
| | | | GQAPRLLIYGASTRATGIPARFSGSGSGTEFTLTISSLQSE |
| | | | DFAVYYCQQYQSWPPLTFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEPKSSDKTHTPPPPPEVQLVES |
| | | | GGGVVQPGGSLRLSCAASGFTFSNYAMTWVRQAPEKG |
| | | | LEWVSSISGSGVSTYYADSVKGRFTISRDNSKNTLYLQM |
| | | | TRLTAEDTAVYFCAKEGSSETDDHYYNVDVWGQGTTVT |
| | | | VSSEPKSSDKTHTCPPCPAPEAAGGPSVFLFPPKPKDTLM |
| | | | ISRTPEVTCVVVDVSHEDPEVKFNWYVDGVEVHNAKTK |
| | | | PREEQYNSTYRVVSVLTVLHQDWLNGKEYKCKVSNKA |
| | | | LPAPIEKTISKAKGQPREPQVYTLPPSREEMTKNQVSLTC |
| | | | LVKGFYPSDIAVEWESNGQPENNYKTTPPVLDSDGSFFL |
| | | | YSKLTVDKSRWQQGNVFSCSVMHEALHNHYTQKSLSLS |
| | | | PGK |
| PR004277 | 155 | Short chain | QVQLVESGGGVVQPGRSLRLSCAASGFTFRSFGMHWVR |
| | | | QAPGKGLEWVAVISYDASNEYYADSVKGRFIISRDNSKD |
| | | | TLYLQMNSLRAGDTAVYYCAKGGGLRWYFAYWGQGTL |
| | | | VTVSSASTKGPSVFPLAPSSKSTSGGTAALGCLVKDYFPE |
| | | | PVTVSWNSGALTSGVHTFPAVLQSSGLYSLSSVVTVPSSS |
| | | | LGTQTYICNVNHKPSNTKVDKKVEPKSC |
| | 156 | Long chain | EIVMTQSPATLSVSPGERATLSCRASQSISSNLGWYQQKP |
| | | | GQAPRLLIYGASTRATGIPARFSGSGSGTEFTLTISSLQSE |
| | | | DFAVYYCQQYQSWPPLTFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEPKSSDKTHTPPPPPEVQLLES |
| | | | GGGLVQPGGSLRLSCAASGFTFSSYAMSWVRQAPGKGL |
| | | | EWVSAISGRGGSTFYADSVRGRFTISRDNSKNTLYLQMN |
| | | | SLRAEDTAMYYCVDGTTGTTDVDYWGQGTLVTVSSEP |
| | | | KSSDKTHTCPPCPAPEAAGGPSVFLFPPKPKDTLMISRTP |
| | | | EVTCVVVDVSHEDPEVKFNWYVDGVEVHNAKTKPREE |
| | | | QYNSTYRVVSVLTVLHQDWLNGKEYKCKVSNKALPAPI |
| | | | EKTISKAKGQPREPQVYTLPPSREEMTKNQVSLTCLVKG |
| | | | FYPSDIAVEWESNGQPENNYKTTPPVLDSDGSFFLYSKLT |
| | | | VDKSRWQQGNVFSCSVMHEALHNHYTQKSLSLSPGK |
| PR005744 | 159 | Short chain | QVQLVESGGGVVQPGRSLRLSCAATGFTFSSYGMYWVR |
| | | | QAPGKGLEWVAAIWNDGSNNYYADSVKGRFTISRDDSK |
| | | | NTLNLQMNSLRAEDTAMYYCARDRLPMASLRYFDWLG |
| | | | VMDAWGQGTSVTVSSASTKGPSVFPLAPSSKSTSGGTA |
| | | | ALGCLVKDYFPEPVTVSWNSGALTSGVHTFPAVLQSSGL |
| | | | YSLSSVVTVPSSSLGTQTYICNVNHKPSNTKVDKKVEPK |
| | | | SC |
| | 160 | Long chain | DIQMTQSPSTLSASVGDRVTITCRASQSISSWLAWYQQK |
| | | | PGKAPKLLIYKASSLESGVPSRFSGSGSGTEFTLTISSLQT |
| | | | DDFATYYCQQYNSYLFTFGQGTKLEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEVQLVETGGGLIQPGGSLRLS |
| | | | CAASGFTVSDSYMTWVRQAPGKGLEWVSVIFSGGRTY |
| | | | YSDSVKGRFTISRDNAKNTLYLQMNSLRAEDTALYYCA |
| | | | RRNYDDTRGTDVFDIWGQGTMVTVSSEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| PR000305 | 141 | Short chain | EVQLVESGGGLVQPGGSLRLSCAASGFNIKDTYIHWVRQ |
| | | | APGKGLEWVARIYPTNGYTRYADSVKGRFTISADTSKNT |
| | | | AYLQMNSLRAEDTAVYYCSRWGGDGFYAMDYWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSC |
| | 142 | Long chain | YADSVKGRFTISRDNSKNTLYLQMNSLRAEDTAVYYCA |
| | | | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEVQLVESGGGLIQPGGSLRLS |
| | | | CAVSGFTVSKNYMSWVRQAPGKGLEWVSVVYSGGSKT |
| | | | RAVPHSPSSFDIWGQGTMVTVSSEPKSCDKTHTCPPCPA |
| | | | PELLGGPSVFLFPPKPKDTLMISRTPEVTCVVVDVSHEDP |
| | | | EVKFNWYVDGVEVHNAKTKPREEQYNSTYRVVSVLTV |
| | | | LHQDWLNGKEYKCKVSNKALPAPIEKTISKAKGQPREP |
| | | | QVYTLPPSREEMTKNQVSLTCLVKGFYPSDIAVEWESNG |
| | | | QPENNYKTTPPVLDSDGSFFLYSKLTVDKSRWQQGNVFS |
| | | | CSVMHEALHNHYTQKSLSLSPGK |
| PR000653 | 141 | Short chain | EVQLVESGGGLVQPGGSLRLSCAASGFNIKDTYIHWVRQ |
| | | | APGKGLEWVARIYPTNGYTRYADSVKGRFTISADTSKNT |
| | | | AYLQMNSLRAEDTAVYYCSRWGGDGFYAMDYWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSC |
| | 143 | Long chain | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECGGGGSGSEVQLVESGGGLIQP |
| | | | GGSLRLSCAVSGFTVSKNYMSWVRQAPGKGLEWVSVV |
| | | | YSGGSKTYADSVKGRFTISRDNSKNTLYLQMNSLRAEDT |
| | | | AVYYCARAVPHSPSSFDIWGQGTMVTVSSEPKSCDKTH |
| | | | TCPPCPAPELLGGPSVFLFPPKPKDTLMISRTPEVTCVVV |
| | | | DVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| PR000654 | 141 | Short chain | EVQLVESGGGLVQPGGSLRLSCAASGFNIKDTYIHWVRQ |
| | | | APGKGLEWVARIYPTNGYTRYADSVKGRFTISADTSKNT |
| | | | AYLQMNSLRAEDTAVYYCSRWGGDGFYAMDYWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSC |
| | 144 | Long chain | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECGGGGSGGGGSGGGGSEVQLV |
| | | | ESGGGLIQPGGSLRLSCAVSGFTVSKNYMSWVRQAPGK |
| | | | GLEWVSVVYSGGSKTYADSVKGRFTISRDNSKNTLYLQ |
| | | | MNSLRAEDTAVYYCARAVPHSPSSFDIWGQGTMVTVSS |
| | | | EPKSCDKTHTCPPCPAPELLGGPSVFLFPPKPKDTLMISR |
| | | | TPEVTCVVVDVSHEDPEVKFNWYVDGVEVHNAKTKPR |
| | | | EEQYNSTYRVVSVLTVLHQDWLNGKEYKCKVSNKALP |
| | | | APIEKTISKAKGQPREPQVYTLPPSREEMTKNQVSLTCLV |
| | | | KGFYPSDIAVEWESNGQPENNYKTTPPVLDSDGSFFLYS |
| | | | KLTVDKSRWQQGNVFSCSVMHEALHNHYTQKSLSLSPG |
| | | | K |
| PR000655 | 141 | Short chain | EVQLVESGGGLVQPGGSLRLSCAASGFNIKDTYIHWVRQ |
| | | | APGKGLEWVARIYPTNGYTRYADSVKGRFTISADTSKNT |
| | | | AYLQMNSLRAEDTAVYYCSRWGGDGFYAMDYWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSC |
| | 145 | Long chain | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECEPKSSDKTHTPPPPPEVQLVES |
| | | | GGGLIQPGGSLRLSCAVSGFTVSKNYMSWVRQAPGKGL |
| | | | EWVSVVYSGGSKTYADSVKGRFTISRDNSKNTLYLQMN |
| | | | SLRAEDTAVYYCARAVPHSPSSFDIWGQGTMVTVSSEPK |
| | | | SCDKTHTCPPCPAPELLGGPSVFLFPPKPKDTLMISRTPE |
| | | | VTCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSREEMTKNQVSLTCLVKGF |
| | | | YPSDIAVEWESNGQPENNYKTTPPVLDSDGSFFLYSKLT |
| | | | VDKSRWQQGNVFSCSVMHEALHNHYTQKSLSLSPGK |
| PR000706 | 141 | Short chain | EVQLVESGGGLVQPGGSLRLSCAASGFNIKDTYIHWVRQ |
| | | | APGKGLEWVARIYPTNGYTRYADSVKGRFTISADTSKNT |
| | | | AYLQMNSLRAEDTAVYYCSRWGGDGFYAMDYWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSC |
| | 149 | Long chain | YSGGSKTYADSVKGRFTISRDNSKNTLYLQMNSLRAEDT |
| | | | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECGGGGSGSEVQLVESGGGLIQP |
| | | | GGSLRLSCAVSGFTVSKNYMSWVRQAPGKGLEWVSVV |
| | | | AVYYCARAVPHSPSSFDIWGQGTMVTVSSGGGGGDKTH |
| | | | TCPPCPAPELLGGPSVFLFPPKPKDTLMISRTPEVTCVVV |
| | | | DVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| PR003335 | 139 | Short chain | EIVMTQSPATLSVSPGERATLSCRASQSISSNLGWYQQKP |
| | | | GQAPRLLIYGASTRATGIPARFSGSGSGTEFTLTISSLQSE |
| | | | DFAVYYCQQYQSWPPLTFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |
| | 150 | Long chain | QVQLVESGGGVVQPGRSLRLSCAASGFTFRSFGMHWVR |
| | | | QAPGKGLEWVAVISYDASNEYYADSVKGRFIISRDNSKD |
| | | | TLYLQMNSLRAGDTAVYYCAKGGGLRWYFAYWGQGTL |
| | | | VTVSSASTKGPSVFPLAPSSKSTSGGTAALGCLVKDYFPE |
| | | | PVTVSWNSGALTSGVHTFPAVLQSSGLYSLSSVVTVPSSS |
| | | | LGTQTYICNVNHKPSNTKVDKKVEPKSCDKTHTCPPCP |
| | | | APEAAGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGEPKSSDKTHTPPPP |
| | | | PRTEVQLVESGGGVVQPGGSLRLSCAASGFTFSNYAMT |
| | | | WVRQAPEKGLEWVSSISGSGVSTYYADSVKGRFTISRDN |
| | | | SKNTLYLQMTRLTAEDTAVYFCAKEGSSETDDHYYNVD |
| | | | VWGQGTTVTVSS |
| PR003550 | 136 | Short chain | DIQMTQSPSTLSASVGDRVTVTCRASQSIYIWLAWYQQK |
| | | | PGKAPNLLIYKASSLETGVPSRFSGSGSGTEFTLTISSLQP |
| | | | DDFATYYCQQYYGSSRTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |
| | 151 | Long chain | EVQLVESGGGLVQPGGSLRLSCAASGFTFSSYWMSWVR |
| | | | QAPGKGLEWVANIKQEGSEKYYVDSVKGRFTISRDNAK |
| | | | NSLYLQMNSLRAEDTAVYYCARDRAVAGAFDIWGQGT |
| | | | MVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPEAAGGPSVFLFPPKPKDTLMISRTPEVTCVVVDVSH |
| | | | EDPEVKFNWYVDGVEVHNAKTKPREEQYNSTYRVVSV |
| | | | LTVLHQDWLNGKEYKCKVSNKALGAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGEPKSSDKTHTPPP |
| | | | PPRTEVQLVESGGGVVQPGGSLRLSCAASGFTFSNYAMT |
| | | | WVRQAPEKGLEWVSSISGSGVSTYYADSVKGRFTISRDN |
| | | | SKNTLYLQMTRLTAEDTAVYFCAKEGSSETDDHYYNVD |
| | | | VWGQGTTVTVSS |
| PR004276 | 139 | Short chain | EIVMTQSPATLSVSPGERATLSCRASQSISSNLGWYQQKP |
| | | | GQAPRLLIYGASTRATGIPARFSGSGSGTEFTLTISSLQSE |
| | | | DFAVYYCQQYQSWPPLTFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |
| | 154 | Long chain | QVQLVESGGGVVQPGRSLRLSCAASGFTFRSFGMHWVR |
| | | | QAPGKGLEWVAVISYDASNEYYADSVKGRFIISRDNSKD |
| | | | TLYLQMNSLRAGDTAVYYCAKGGGLRWYFAYWGQGTL |
| | | | VTVSSASTKGPSVFPLAPSSKSTSGGTAALGCLVKDYFPE |
| | | | PVTVSWNSGALTSGVHTFPAVLQSSGLYSLSSVVTVPSSS |
| | | | LGTQTYICNVNHKPSNTKVDKKVEPKSCDKTHTCPPCP |
| | | | APEAAGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGEPKSSDKTHTPPPP |
| | | | PRTEVQLLESGGGLVQPGGSLRLSCAASGFTFSSYAMSW |
| | | | VRQAPGKGLEWVSAISGRGGSTFYADSVRGRFTISRDNS |
| | | | KNTLYLQMNSLRAEDTAMYYCVDGTTGTTDVDYWGQ |
| | | | GTLVTVSS |
| PR004268 | 136 | Short chain | DIQMTQSPSTLSASVGDRVTVTCRASQSIYIWLAWYQQK |
| | | | PGKAPNLLIYKASSLETGVPSRFSGSGSGTEFTLTISSLQP |
| | | | DDFATYYCQQYYGSSRTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |
| | 152 | Long chain | EVQLVESGGGVVQPGGSLRLSCAASGFTFSNYAMTWVR |
| | | | QAPEKGLEWVSSISGSGVSTYYADSVKGRFTISRDNSKN |
| | | | TLYLQMTRLTAEDTAVYFCAKEGSSETDDHYYNVDVW |
| | | | GQGTTVTVSSGGGGSGGGGSGGGGSEVQLVESGGGLVQ |
| | | | PGGSLRLSCAASGFTFSSYWMSWVRQAPGKGLEWVANI |
| | | | KQEGSEKYYVDSVKGRFTISRDNAKNSLYLQMNSLRAE |
| | | | DTAVYYCARDRAVAGAFDIWGQGTMVTVSSASTKGPSV |
| | | | FPLAPSSKSTSGGTAALGCLVKDYFPEPVTVSWNSGALT |
| | | | SGVHTFPAVLQSSGLYSLSSVVTVPSSSLGTQTYICNVNH |
| | | | KPSNTKVDKKVEPKSCDKTHTCPPCPAPEAAGGPSVFLF |
| | | | PPKPKDTLMISRTPEVTCVVVDVSHEDPEVKFNWYVDG |
| | | | VEVHNAKTKPREEQYNSTYRVVSVLTVLHQDWLNGKE |
| | | | YKCKVSNKALPAPIEKTISKAKGQPREPQVYTLPPSREE |
| | | | MTKNQVSLTCLVKGFYPSDIAVEWESNGQPENNYKTTPP |
| | | | VLDSDGSFFLYSKLTVDKSRWQQGNVFSCSVMHEALHN |
| | | | HYTQKSLSLSPGK |
| PR004278 | 139 | Short chain | EIVMTQSPATLSVSPGERATLSCRASQSISSNLGWYQQKP |
| | | | GQAPRLLIYGASTRATGIPARFSGSGSGTEFTLTISSLQSE |
| | | | DFAVYYCQQYQSWPPLTFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |
| | 157 | Long chain | EVQLVESGGGVVQPGGSLRLSCAASGFTFSNYAMTWVR |
| | | | QAPEKGLEWVSSISGSGVSTYYADSVKGRFTISRDNSKN |
| | | | TLYLQMTRLTAEDTAVYFCAKEGSSETDDHYYNVDVW |
| | | | GQGTTVTVSSGGGGSGGGGSGGGGSQVQLVESGGGVV |
| | | | QPGRSLRLSCAASGFTFRSFGMHWVRQAPGKGLEWVAV |
| | | | ISYDASNEYYADSVKGRFIISRDNSKDTLYLQMNSLRAG |
| | | | DTAVYYCAKGGGLRWYFAYWGQGTLVTVSSASTKGPS |
| | | | VFPLAPSSKSTSGGTAALGCLVKDYFPEPVTVSWNSGAL |
| | | | TSGVHTFPAVLQSSGLYSLSSVVTVPSSSLGTQTYICNVN |
| | | | HKPSNTKVDKKVEPKSCDKTHTCPPCPAPEAAGGPSVFL |
| | | | FPPKPKDTLMISRTPEVTCVVVDVSHEDPEVKFNWYVD |
| | | | GVEVHNAKTKPREEQYNSTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPP |
| | | | VLDSDGSFFLYSKLTVDKSRWQQGNVFSCSVMHEALHN |
| | | | HYTQKSLSLSPGK |
| PR000701 | 148 | Polypeptide | DIQMTQSPSTLSASVGDRVTVTCRASQSIYIWLAWYQQK |
| | | chain 1 | PGKAPNLLIYKASSLETGVPSRFSGSGSGTEFTLTISSLQP |
| | | | DDFATYYCQQYYGSSRTFGQGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGECQVQLQQWGAGLLKPSETLSL |
| | | | TCAVYGGSFSGYYWSWIRQPPGKGLEWIGEINHSGSTDS |
| | | | NPSLKGRVTFSVDTSKNQFSLKLNSVTAADTAVYYCARL |
| | | | TGPFDYWGQGTLVTVSSASTKGPSVFPLAPCSRSTSESTA |
| | | | ALGCLVKDYFPEPVTVSWNSGALTSGVHTFPAVLQSSGL |
| | | | YSLSSVVTVPSSSLGTKTYTCNVDHKPSNTKVDKRVESK |
| | | | YGPPCPPCPAPEFLGGPSVFLFPPKPKDTLMISRTPEVTCV |
| | | | VVDVSQEDPEVQFNWYVDGVEVHNAKTKPREEQFNST |
| | | | YRVVSVLTVLHQDWLNGKEYKCKVSNKGLPSSIEKTISK |
| | | | AKGQPREPQVYTLPPSQEEMTKNQVSLTCLVKGFYPSDI |
| | | | AVEWESNGQPENNYKTTPPVLDSDGSFFLYSRLTVDKSR |
| | | | WQEGNVFSCSVMHEALHNHYTQKSLSLSLGK |
| | 147 | Polypeptide | EVQLVESGGGLVQPGGSLRLSCAASGFTFSSYWMSWVR |
| | | chain 2 | QAPGKGLEWVANIKQEGSEKYYVDSVKGRFTISRDNAK |
| | | | NSLYLQMNSLRAEDTAVYYCARDRAVAGAFDIWGQGT |
| | | | MVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSC |
| | 146 | Polypeptide | EIVMTQSPATLSVSPGERATLSCRASQSISSILAWYQQKP |
| | | chain 3 | GQAPRLLIYGASTRATGIPARFSGSGSGTEFTLTISSLQSE |
| | | | DFAVYYCQQYYNWPLTFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACEV |
| | | | THQGLSSPVTKSFNRGEC |

### Example 4. PD-L1×4-1BB Bispecific Binding Proteins

In this example, we constructed PD-L1×4-1BB bispecific binding proteins with the Fab-HCAb, IgG-VH, VH-IgG or FIT-Ig structure targeting PD-L1 and 4-1BB to improve anti-tumor efficacy and safety through one or more mechanisms of action. Firstly, PD-L1×4-1BB can activate T cells by blocking the PD-1/PD-L1 signaling pathway. Secondly, the PD-L1 molecule highly expressed on the surface of the tumor cells can promote the crosslinking and trimerization of 4-1BB molecules on the surface of the T cells and activate the downstream signaling pathway using PD-L1×4-1BB, thereby promoting the activation and proliferation of the T cells. Thirdly, PD-L1×4-1BB-mediated T cell activation is limited to the tumor microenvironment, so that the toxic and side effects caused by over-activation of the T cells in normal tissues by monoclonal antibodies similar to Urelumab can be avoided.

### Example 4.1 Acquisition of Anti-PD-L1 IgG Antibodies and Anti-4-1BB IgG or HCAb Antibodies

### Example 4.1.1 Acquisition of Fully Human Anti-PD-L1 IgG Antibodies

The Harbour H2L2 mouse (Harbour Antibodies BV) is a transgenic mouse carrying an immune repertoire of human immunoglobulins that produces antibodies with intact human antibody variable domains and rat constant domains.

Harbour H2L2 mice were subjected to multiple rounds of immunization with a soluble recombinant human PD-L1 protein (NovoProtein, #CM06). When the titer of the PD-L1-specific antibody in the serum of mice was detected to reach a certain level, spleen cells of the mice were taken and fused with a myeloma cell line to obtain hybridoma cells. After multiple rounds of screening and cloning of the hybridoma cells, several monoclonal antibody molecules specifically recognizing PD-L1 were identified. Those monoclonal antibodies were further identified, and several candidate antibody molecules were preferentially selected according to parameters such as the binding ability to human PD-L1, the binding ability to cynomolgus monkey PD-L1, and the ability to inhibit the binding of PD-L1 to PD-1. The candidate antibody molecules were then subjected to sequence analysis and optimization to obtain several variant sequences. The VL and VH sequences of the antibody were fused to the corresponding human κ light chain constant region and IgG1 heavy chain constant region sequences and expressed to obtain recombinant fully human antibody molecules.

The sequences of the recombinant fully human anti-PD-L1 IgG antibody PR000265 are shown in Table 6.

### Example 4.1.2 Acquisition of Fully Human Anti-4-1BB IgG Antibodies

The Harbour H2L2 mouse (Harbour Antibodies BV) is a transgenic mouse carrying an immune repertoire of human immunoglobulins that produces antibodies with intact human antibody variable domains and rat constant domains.

Harbour H2L2 mice were subjected to multiple rounds of immunization with a soluble recombinant human 4-1BB-Fc fusion protein (GenScript Biotech). When the titer of the 4-1BB-specific antibody in the serum of mice was detected to reach a certain level, spleen cells of the mice were taken and fused with a myeloma cell line to obtain hybridoma cells. After multiple rounds of screening and cloning of the hybridoma cells, several monoclonal antibody molecules specifically recognizing 4-1BB were identified. Those monoclonal antibodies were further identified, and several candidate antibody molecules were preferentially selected according to parameters such as the binding ability to human 4-1BB, the binding ability to cynomolgus monkey 4-1BB, and the T cell activation ability. The candidate antibody molecules were then subjected to sequence analysis and optimization to obtain several variant sequences. The VL and VH sequences of the antibody were fused to the corresponding human κ light chain constant region and IgG1 heavy chain constant region sequences and expressed to obtain recombinant fully human antibody molecules.

The sequences of the recombinant fully human anti-4-1BB IgG antibody PR000197 are shown in Table 6.

### Example 4.1.3 Acquisition of Fully Human Anti-4-1BB HCAb Antibodies

The Harbour HCAb mouse (Harbour Antibodies BV, WO2010/109165A2) is a transgenic mouse carrying an immune repertoire of human immunoglobulins, capable of producing heavy chain-only antibodies that are only half the molecular weight of conventional IgG antibodies. The antibodies produced have only human antibody heavy chain variable domains and mouse Fc constant domains.

Harbour HCAb mice were subjected to multiple rounds of immunization with a soluble recombinant human 4-1BB-Fc fusion protein (provided by ChemPartner) or human 4-1BB-overexpressing NIH-3T3 cells (provided by ChemPartner). When the titer of the 4-1BB-specific antibody in the serum of mice was detected to reach a certain level, spleen cells of the mice were taken, from which B cells were isolated, and the CD138-positive plasma cells were sorted using a mouse plasma cell isolation kit (Miltenyi, #130-092-530). The human VH gene was amplified from plasma cells using conventional molecular biology techniques, and the amplified human VH gene fragments were constructed into mammalian cell expression plasmid pCAG vectors encoding the sequence of the heavy chain Fc of the human IgG1 antibody. Mammal host cells (e.g., human embryonic kidney cell HEK293) were transfected with the plasmids and allowed to express antibodies to obtain a supernatant with fully human HCAb antibodies. Positive HCAb antibodies were identified by testing the supernatant with HCAb antibodies for binding to CHO-K1 cell CHO-K1/hu4-1BB highly expressing human 4-1BB by FACS. Those HCAb antibodies were further identified, and several candidate HCAb antibody molecules were preferentially selected according to parameters such as the binding ability to human 4-1BB, the binding ability to cynomolgus monkey 4-1BB, and the T cell activation ability.

The sequences of the recombinant fully human anti-4-1BB HCAb antibody PR001760 are shown in Table 6.

### Example 4.2 Construction of PD-L1×4-1BB Bispecific Binding Proteins

In one aspect, in this example, anti-PD-L1×4-1BB bispecific binding proteins with the Fab-HCAb structure (FIG. 1 structure (1): Fab(CL)-VH-Fc) described in Example 1.1.1, PR004270 and PR007164, and an anti-PD-L1×4-1BB bispecific binding protein with the Fab-HCAb structure (FIG. 1 structure (2): Fab(CH1)-VH-Fc) described in Example 1.1.2, PR007163, were constructed using Fab of the anti-PD-L1 IgG antibody PR000265 and VH of the anti-4-1BB HCAb antibody PR001760. The molecular designs of PR004270, PR007163 and PR007164 are shown in Table 4, with the corresponding sequence numbers shown in Table 7. The molecules were prepared and analyzed by the method described in Example 2, with the results summarized in Table 9. As shown in Table 9, PR007164 (structure (1)) and PR004270 had a significantly higher yield after purification than PR007163 (structure (2)).

In another aspect, in this example, an anti-PD-L1×4-1BB bispecific binding protein with the IgG-VH structure described in Example 1.1.3, PR003550, was also constructed using Fab of the anti-PD-L1 IgG antibody PR000265 and VH of the anti-4-1BB HCAb antibody PR001760. The molecular design of PR003550 is shown in Table 5, with the corresponding sequence numbers shown in Table 7. The molecule was prepared and analyzed by the method described in Example 2, with the results summarized in Table 10.

In another aspect, in this example, an anti-PD-L1×4-1BB bispecific binding protein with the VH-IgG structure described in Example 1.1.4, PR004268, was also constructed using Fab of the anti-PD-L1 IgG antibody PR000265 and VH of the anti-4-1BB HCAb antibody PR001760. The molecular design of PR004268 is shown in Table 5, with the corresponding sequence numbers shown in Table 7. The molecule was prepared and analyzed by the method described in Example 2, with the results summarized in Table 10.

In another aspect, in this example, an anti-PD-L1×4-1BB bispecific binding protein with the FIT-Ig structure described in Example 1.2.1, PR000701, was also constructed using Fab of the anti-PD-L1 IgG antibody PR000265 and Fab of the anti-4-1BB IgG antibody PR000197. The molecular design of PR000701 is shown in Table 5, with the corresponding sequence numbers shown in Table 7. The molecule was prepared and analyzed by the method described in Example 2, with the results summarized in Table 10.

### Example 4.3 Binding to 4-1BB

In this example, the binding ability of the binding proteins to a CHO-K1 cell strain CHO-K1/hu 4-1BB (GenScript Biotech, M00538) cells highly expressing human 4-1BB was determined by flow cytometry FACS. Specifically, the cells were digested and resuspended in a complete medium, and the cell density was adjusted to 2×10⁶ cells/mL. Thereafter, the cells were seeded in a 96-well V-bottom plate (Corning, #3894) at 100 μL/well (2×10⁵ cells/well) and centrifuged at 4° C. for 5 min, and the supernatant was discarded. Then, the binding proteins diluted in a gradient were added to the 96-well plate at 100 μL/well, and the mixture was mixed well, wherein the binding proteins may have a total of 12 concentrations obtained by a 3-fold gradient dilution from the highest final concentration of 200 nM. hIgG1 iso (CrownBio, #C0001) was used as an isotype control. The cells were incubated at 4° C. for 1 h away from light. Then, the cells in each well were rinsed twice with 100 μL of pre-cooled FACS buffer (PBS buffer containing 0.5% BSA) and centrifuged at 500 g at 4° C. for 5 min, and the supernatant was discarded. Thereafter, a fluorescent secondary antibody (Goat human IgG (H+L) Alexa Fluor 488 conjugation, Thermo, #A11013, diluted in a 1:1000 ratio) was added at 100 μL/well, and the plate was incubated at 4° C. for 1 h away from light. Then, the cells in each well were then rinsed twice with 200 μL of pre-cooled FACS buffer and centrifuged at 500 g at 4° C. for 5 min, and then the supernatant was discarded. Finally, a pre-cooled FACS buffer was added at 200 μL/well to resuspend the cells. Fluorescence signal values were read using a BD FACS CANTOII flow cytometer or an ACEA NovoCyte flow cytometer, and the data were processed and analyzed using FlowJo v10 (FlowJo, LLC) software.

The data were processed and analyzed by plotting using GraphPad Prism 8 software, and binding curves of the binding proteins to target cells, EC₅₀ values and other parameters were obtained through four-parameter nonlinear fitting.

In this example, the anti-4-1BB monoclonal antibody urelumab (protein No. PR000628) or the anti-4-1BB HCAb antibody PR001760 was used as a positive control molecule.

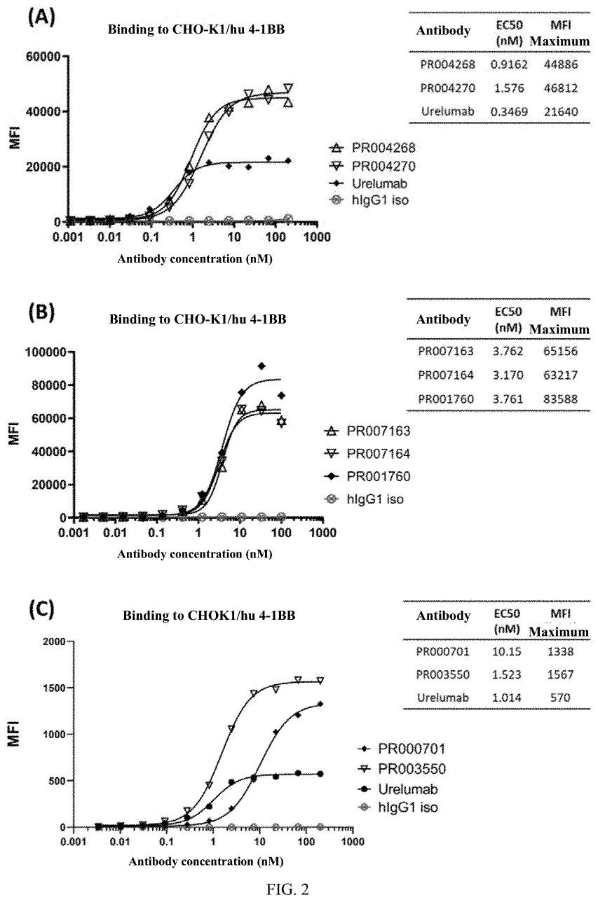

As shown in FIG. 2, the PD-L1×4-1BB bispecific binding proteins (PR004270, PR004268, and PR003550) showed comparable binding ability to 4-1BB, and was superior to the positive control urelumab in the maximum MFI value and to the molecule with the FIT-Ig structure PR000701 in the EC₅₀ value. The PD-L1×4-1BB bispecific binding proteins (PR007163 and PR007164) had comparable binding ability to 4-1BB to the parent monoclonal antibody PR001760.

### Example 4.4 Binding to PD-L1

In this example, the binding ability of the binding proteins to a CHO-K1 cell strain CHO-K1/hPD-L1 (GenScript Biotech, M00543) highly expressing human PD-L1 was determined by flow cytometry FACS. Specifically, the cells were digested and resuspended in a complete medium, and the cell density was adjusted to 1×10⁶ cells/mL. Thereafter, the cells were seeded in a 96-well V-bottom plate (Corning, #3894) at 100 μL/well and centrifuged at 4° C. for 5 min, and the supernatant was discarded. Then, the binding proteins diluted in a gradient were added to the 96-well plate at 100 μL/well, and the mixture was mixed well, wherein the binding proteins may have a total of 12 concentrations obtained by a 3-fold gradient dilution from the highest final concentration of 200 nM. hIgG1 iso (CrownBio, #C0001) was used as an isotype control. The cells were incubated at 4° C. for 1 h away from light. Then, the cells in each well were rinsed twice with 100 μL of pre-cooled FACS buffer (PBS buffer containing 0.5% BSA) and centrifuged at 500 g at 4° C. for 5 min, and the supernatant was discarded. Thereafter, a fluorescent secondary antibody (Goat human IgG (H+L) Alexa Fluor 488 conjugation, Thermo, #A11013, diluted in a 1:1000 ratio) was added at 100 μL/well, and the plate was incubated at 4° C. for 1 h away from light. Then, the cells in each well were then rinsed twice with 200 μL of pre-cooled FACS buffer and centrifuged at 500 g at 4° C. for 5 min, and then the supernatant was discarded. Finally, a pre-cooled FACS buffer was added at 200 μL/well to resuspend the cells. Fluorescence signal values were read using a BD FACS CANTOII flow cytometer or an ACEA NovoCyte flow cytometer, and the data were processed and analyzed using FlowJo v10 (FlowJo, LLC) software.

The data were processed and analyzed by plotting using GraphPad Prism 8 software, and binding curves of the binding proteins to target cells, EC₅₀ values and other parameters were obtained through four-parameter nonlinear fitting.

In this example, the anti-PD-L1 monoclonal antibody PR000265 was used as a positive control molecule, and was also the parent monoclonal antibody of the PD-L1 end of PD-L1×4-1BB.

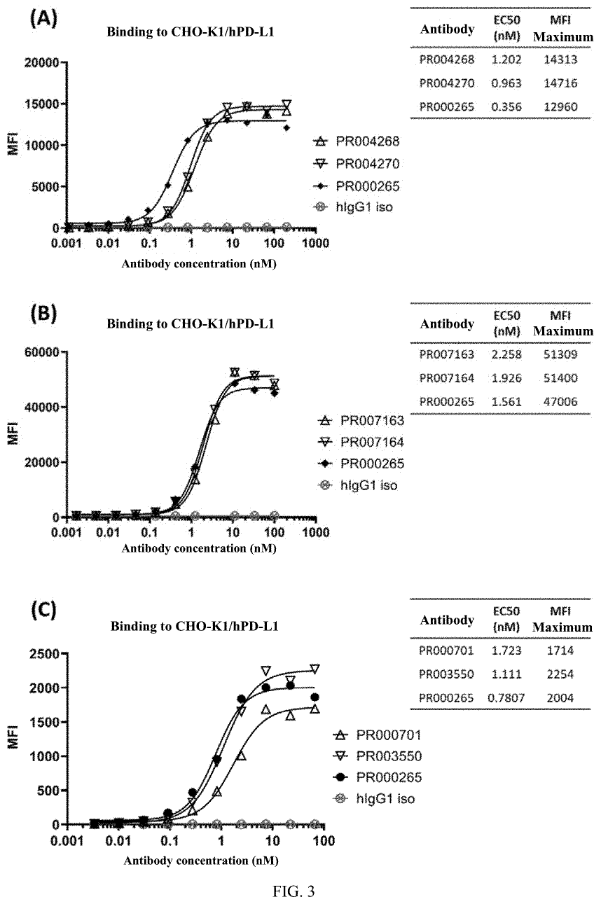

As shown in FIG. 3, the molecule with the Fab-HCAb structure (PR004270) and the molecule with the VH-IgG structure (PR004268) had similar binding ability to PD-L1 to the parent monoclonal antibody PR000265, and had a slightly poorer EC₅₀ value but a higher maximum MFI value for binding to PD-L1 than the parent monoclonal antibody. The molecule with the IgG-VH structure (PR003550) had similar binding ability to PD-L1 to the parent monoclonal antibody PR000265, and had slightly superior EC₅₀ value and maximum MFI value to the molecule with the FIT-Ig structure (PR000701). The molecules with the Fab-HCAb structure (PR007163 and PR007164) had comparable binding ability to PD-L1 to the parent monoclonal antibody PR000265.

### Example 4.5 Mixed Lymphocyte Reaction (MLR)

This example is intended to investigate the T cell activation effect of PD-L1×4-1BB bispecific binding proteins by the mixed lymphocyte reaction (MLR).

In the first step, monocytes were isolated from PBMC cells (MT-Bio) of a first donor using CD14 magnetic beads (Meltenyi, #130-050-201) by referring to the instructions of the relevant kit. Then, 50 ng/mL of recombinant human IL-4 (PeproTech, #200-02-A) and 100 ng/mL of recombinant human GM-CSF (PeproTech, #300-03-A) were added, and after 7 days of induction at 37° C., immature dendritic cells (iDC cells) were obtained. 1 μg/mL lipopolysaccharide (LPS, Sigma, #L6529) was then added, and after 24 h of induction, mature dendritic cells (mDC cells) were obtained. In the second step, T lymphocytes were isolated from PBMC cells (MT-Bio) of a second donor using a T cell isolation kit (Meltenyi, #130-096-535). In the third step, the obtained T cells and mDC cells were seeded in a 96-well plate (T cells at 1×10⁵/well and mDC cells at 2×10⁴/well) at a ratio of 5:1. Then, binding proteins at different concentrations were added at 50 μL/well, wherein the binding protein concentration may be the final concentration of (10 nM, 1 nM); and 3 duplicate wells were set for each concentration. hIgG1 iso (CrownBio, #C0001) or a blank well was used as a control. The cells were incubated in an incubator at 37° C. with 5% CO₂ for 5 days. In the fourth step, supernatants on day 4 and on day 5 were each collected. The IL-2 concentration in the supernatant on day 4 was determined using an IL-2 ELISA kit (Thermo, #88-7025-88), and the IFN-γ concentration in the supernatant on day 5 was determined using an IFN-γ ELISA kit (Thermo, #88-7316-77). The ELISA assay was performed by referring to the instructions of relevant kit. The data were processed and analyzed by plotting using GraphPad Prism 8 software.

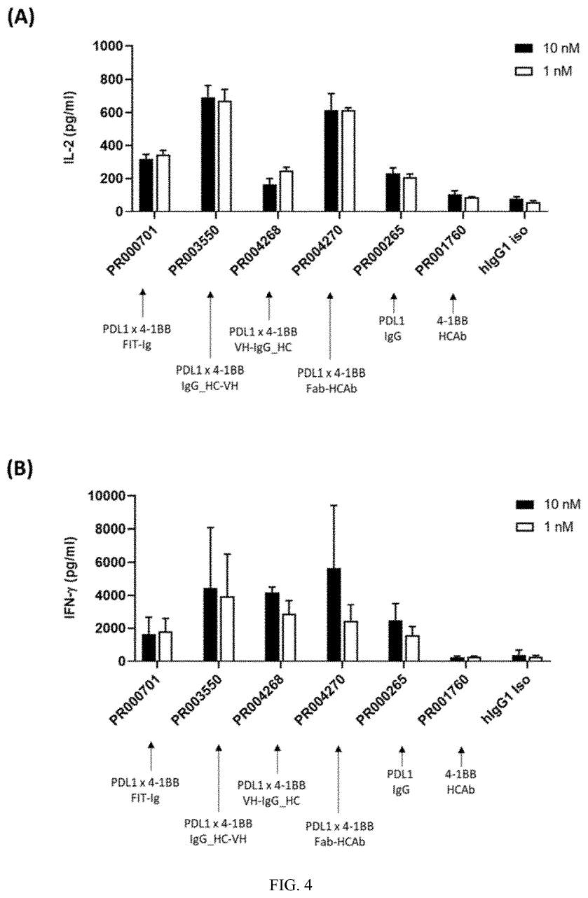

As shown in FIG. 4, in the MLR assay, the anti-4-1BB monoclonal antibody (PR001760) had a limited T cell activation effect and very weak cytokine (IFN-γ and IL-2) production ability; however, the anti-PD-L1 monoclonal antibody (PR000265) had a relatively significant activation effect. In another aspect, the PD-L1×4-1BB bispecific binding proteins were able to further improve the function of the T cells, and were superior to the anti-PD-L1 monoclonal antibody. Moreover, the molecule with the IgG-VH structure (PR003550) and the molecule with the Fab-HCAb structure (PR004270) were able to stimulate the production of more cytokines by T cells as compared to the molecule with the FIT-Ig structure (PR000701).

### Example 4.6 T Cell Specific Activation Mediated by Target Cells

This example is intended to investigate the T cell activation activity of the PD-L1×4-1BB bispecific binding proteins by binding to 4-1BB in the presence of target cells. The target cells may be CHO-K1/hPD-L1 (GenScript Biotech, M00543) highly expressing human PD-L1; and the effector cells may be isolated human PBMC or T cells.

Specifically, a 96-well plate (Corning, #3599) was coated firstly with 0.3 μg/mL anti-CD3 antibody OKT3 (Thermo, #16-0037-81) at 100 μL/well. Thereafter, the density of human T cells (isolated from human PBMCs with a T cell isolation kit (Miltenyi, #130-096-535)) was adjusted to 2×10⁶ cells/mL, and the density of target cells was adjusted to 3×10⁵ cells/mL. The two cell suspensions were each seeded in the 96-well plate at 50 μL/well, with a final effector-to-target ratio of 20:3. Then, binding proteins at different concentrations were added at 100 μL/well, wherein the binding protein concentration may be the final concentration of (10 nM, 1 nM); and 2 duplicate wells were set for each concentration. hIgG1 iso (CrownBio, #C0001) and hIgG4 iso (CrownBio, #C0045) were used as a control. The 96-well plate was incubated in an incubator at 37° C. with 5% CO₂ for 3 days. Supernatants after 48 h and 72 h of culture were each collected. The IL-2 concentration in the supernatant after 48 h was determined using an IL-2 ELISA kit (Thermo, #88-7025-88), and the IFN-γ concentration in the supernatant after 72 h was determined using an IFN-γ ELISA kit (Thermo, #88-7316-77). The ELISA assay was performed by referring to the instructions of relevant kit. The data were processed and analyzed by plotting using GraphPad Prism 8 software.

As shown in FIG. 8, in a system where the target cells CHO-K1/hPD-L1 and the T cells were mixed, the non-crosslinking-dependent anti-4-1BB monoclonal antibody Urelumab was able activate the T cells to release IFN-γ; the molecule with the Fab-HCAb structure (PR004270) has the strongest T cell activation ability with the IFN-γ level higher than that of Urelumab and bispecific molecules with other structures (e.g., PR003550 and PR000701). Overall, the T cell activation ability is ranked as follows: PR004270>PR003550>Urelumab=PR000701>PR004268.

### Example 4.7 Structural Simulation

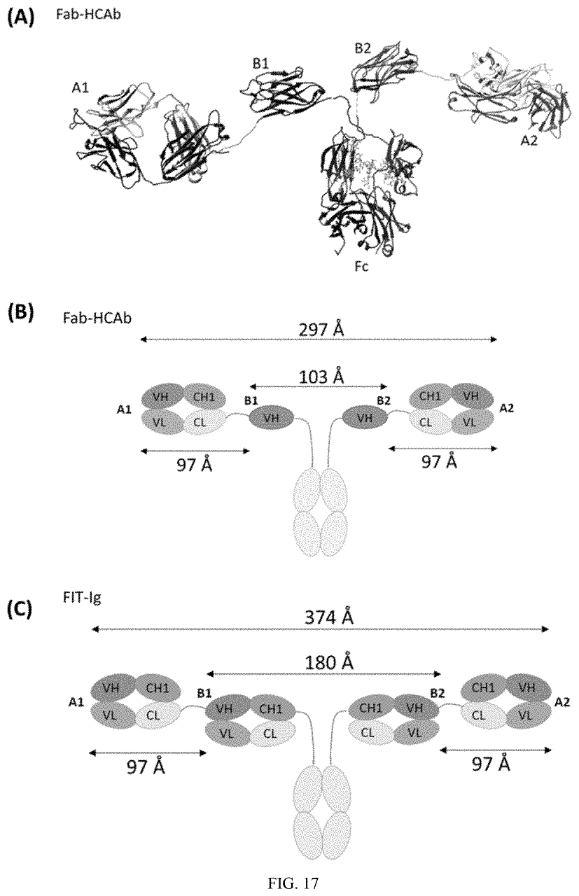

In Example 4.5 and Example 4.6, the molecule with the Fab-HCAb structure (PR004270) showed stronger T cell activation ability than the molecule with the FIT-Ig structure (PR000701). In order to further investigate the differences between the Fab-HCAb structure and the FIT-Ig structure, a three-dimensional structure model of Fab-HCAb (structure (1)) (FIG. 17 (A)) and a three-dimensional structure model of FIT-Ig (structure (5)) were predicted by a homology modeling technique using the known crystal structure of the human IgG1 full-length antibody (PDB accession number: 1HZH) in this example, and on this basis, the relative distances between the different antigen-binding sites (FIGS. 17 (B) and (C)) were measured. In both structure models, the protein functional region A and the protein functional region B were linked via a linker peptide GS-7 in a length of 7 amino acids (SEQ ID NO: 163).

As shown in FIG. 17, the Fab-HCAb structure was more compact. In the most extended model of the Fab-HCAb structure, the distance between the two VH ends (B1 and B2) was about 10 nm and the distance between the two Fab ends (A1 and A2) was about 30 nm; accordingly, in the FIT-Ig structure, the distance between B1 and B2 was about 18 nm, and the distance between A1 and A2 was about 37 nm. In bispecific binding proteins targeting 4-1BB, this more compact Fab-HCAb structure may be more favorable for trimerization and clustering on the cell surface of 4-1BB, which in turn activates downstream signaling.

### Example 5. B7H4×4-1BB Bispecific Binding Proteins

In this example, we constructed B7H4×4-1BB bispecific binding proteins with the Fab-HCAb, IgG-VH or VH-IgG structure targeting B7H4 and 4-1BB to improve anti-tumor efficacy and safety through one or more mechanisms of action. Firstly, B7H4×4-1BB can activate T cells by relieving the negative regulatory signals of B7H4. Secondly, B7H4×4-1BB is enriched in tumor tissues with highly expressed B7H4, and immune cells and tumor cells are combined together through B7H4×4-1BB in a tumor microenvironment to promote the formation of immune synapses; meanwhile, the B7H4 molecules highly expressed on the surface of the tumor cells can promote the crosslinking of 4-1BB molecules on the surface of the T cells through B7H4×4-1BB, activate the downstream signaling pathway and provide costimulatory signals, thereby promoting the activation and proliferation of T cells and improving the anti-tumor activity. Thirdly, B7H4×4-1BB can only mediate T cell activation using target cells in a tumor microenvironment to avoid the toxic and side effects caused by over-activation of T cells in normal tissues by monoclonal antibodies similar to Urelumab.

### Example 5.1 Acquisition of Anti-B7H4 IgG Antibodies and Anti-4-1BB HCAb Antibodies

### Example 5.1.1 Acquisition of Fully Human Anti-B7H4 IgG Antibodies

The Harbour H2L2 mouse (Harbour Antibodies BV) is a transgenic mouse carrying an immune repertoire of human immunoglobulins that produces antibodies with intact human antibody variable domains and rat constant domains.

Harbour H2L2 mice were subjected to multiple rounds of immunization with a soluble recombinant human B7H4-mFc fusion protein (Sino Biological Inc., #10738-H05H). When the titer of the B7H4-specific antibody in the serum of mice was detected to reach a certain level, spleen cells of the mice were taken and fused with a myeloma cell line to obtain hybridoma cells. After multiple rounds of screening and cloning of the hybridoma cells, several monoclonal antibody molecules specifically recognizing B7H4 were identified. The monoclonal antibodies were further identified, and several candidate antibody molecules were preferentially selected according to parameters such as the binding ability to human B7H4, the binding ability to cynomolgus monkey B7H4, and the internalization ability of target cell receptors. The candidate antibody molecules were then subjected to sequence analysis and optimization to obtain several variant sequences. The VL and VH sequences of the antibody were fused to the corresponding human κ light chain constant region and IgG1 heavy chain constant region sequences and expressed to obtain recombinant fully human antibody molecules.

The sequences of the recombinant fully human anti-B7H4 IgG antibody PR002408 are shown in Table 6.

### Example 5.1.2 Acquisition of Fully Human Anti-4-1BB HCAb Antibodies

The fully human anti-4-1BB HCAb antibody PR001760 (Table 6) used in this example was derived from Harbour HCAb mice, and was found as described in Example 4.1.3.

### Example 5.2 Construction of B7H4×4-1BB Bispecific Binding Proteins

In one aspect, in this example, an anti-B7H4×4-1BB bispecific binding protein with the Fab-HCAb structure described in Example 1.1.1, PR004279, was constructed using Fab of the anti-B7H4 IgG antibody PR002408 and VH of the anti-4-1BB HCAb antibody PR001760. The molecular design of PR004279 is shown in Table 4, with the corresponding sequence numbers shown in Table 7. The molecule was prepared and analyzed by the method described in Example 2, with the results summarized in Table 9.

In another aspect, in this example, an anti-B7H4×4-1BB bispecific binding protein with the IgG-VH structure described in Example 1.1.3, PR003335, was also constructed using Fab of the anti-B7H4 IgG antibody PR002408 and VH of the anti-4-1BB HCAb antibody PR001760. The molecular design of PR003335 is shown in Table 5, with the corresponding sequence numbers shown in Table 7. The molecule was prepared and analyzed by the method described in Example 2, with the results summarized in Table 10.

In another aspect, in this example, an anti-B7H4×4-1BB bispecific binding protein with the VH-IgG structure described in Example 1.1.4, PR004278, was also constructed using Fab of the anti-B7H4 IgG antibody PR002408 and VH of the anti-4-1BB HCAb antibody PR001760. The molecular design of PR004278 is shown in Table 5, with the corresponding sequence numbers shown in Table 7. The molecule was prepared and analyzed by the method described in Example 2, with the results summarized in Table 10.

### Example 5.3 Binding to 4-1BB

In this example, the binding ability of the binding proteins to a CHO-K1 cell strain CHO-K1/hu 4-1BB (GenScript Biotech, M00538) cells highly expressing human 4-1BB was determined by the method described in Example 4.3.

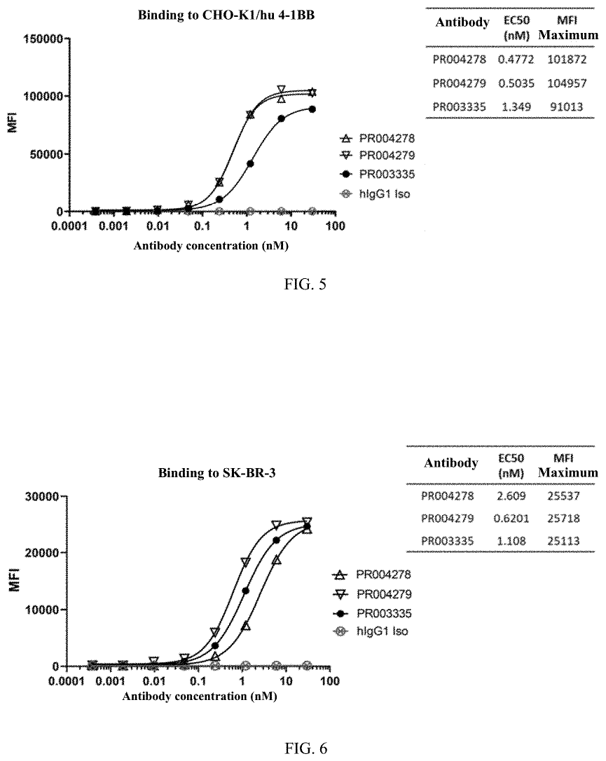

As shown in FIG. 5, the B7H4×4-1BB bispecific binding proteins (PR004279, PR004278 and PR003335) were all able to bind to 4-1BB; moreover, the molecule with the Fab-HCAb structure (PR004279) and the molecule with the VH-IgG structure (PR004278) had superior binding ability to 4-1BB to the molecule with the IgG-VH structure (PR003335).

### Example 5.4 Binding to B7H4

In this example, the binding ability of the binding proteins to a tumor cell line SK-BR-3 (ATCC, HTB-30) highly expressing human B7H4 was determined by flow cytometry FACS. Specifically, SK-BR-3 cells were digested and resuspended in a complete medium, and the cell density was adjusted to 2×10⁶ cells/mL. Then, the cells were seeded in a 96-well V-bottom plate (Corning, #3894) at 50 μL/well. Then, binding proteins at a total of 8 concentrations obtained by a 5-fold gradient dilution were added at 50 μL/well, and the mixture was mixed well. hIgG1 iso (CrownBio, #C0001) was used as an isotype control. The cells were incubated at 4° C. for 2 h away from light. Then, the cells in each well were then rinsed twice with 100 μL of pre-cooled PBS buffer and centrifuged at 500 g at 4° C. for 5 min, and then the supernatant was discarded. Thereafter, a fluorescent secondary antibody (Alexa Fluor 647-conjugated AffiniPure Goat Anti-Human IgG, Fcγ Fragment Specific, Jackson ImmunoResearch, #109-605-098, diluted in a 1:1000 ratio) was added at 100 μL/well, and the plate was incubated at 4° C. for 1 h away from light. Then, the cells in each well were then rinsed twice with 100 μL of pre-cooled PBS buffer and centrifuged at 500 g at 4° C. for 5 min, and then the supernatant was discarded. Finally, a pre-cooled FACS buffer (PBS buffer containing 0.5% BSA) was added at 200 μL/well to resuspend the cells. Fluorescence signal values were read using a BD FACS CANTOII flow cytometer or an ACEA NovoCyte flow cytometer, and the data were processed and analyzed using FlowJo v10 (FlowJo, LLC) software.

The data were processed and analyzed by plotting using GraphPad Prism 8 software, and binding curves of the binding proteins to target cells, EC₅₀ values and other parameters were obtained through four-parameter nonlinear fitting.

As shown in FIG. 6, the B7H4×4-1BB bispecific binding proteins (PR004279, PR004278 and PR003335) were all able to bind to B7H4; moreover, the molecule with the Fab-HCAb structure (PR004279) had slightly superior binding ability to B7H4 to the molecule with other structures.

### Example 5.5 T Cell Specific Activation Mediated by Target Cells

This example is intended to investigate the T cell activation activity of the B7H4×4-1BB bispecific binding proteins by binding to 4-1BB in the presence of target cells. The target cells may be SK-BR-3 cells (ATCC, HTB-30) highly expressing human B7H4; and the effector cells may be isolated human PBMC or T cells.

Specifically, anti-CD3 antibody OKT3 (Thermo, #16-0037-81) was firstly used for coating a 96-well plate (Corning, #3799). Then, the density of human T cells was adjusted to 3×10⁶ cells/mL, and the density of target cells was adjusted to 3×10⁵ cells/mL. The two cell suspensions were each seeded in a 96-well plate at 50 μL/well, with a final effector-to-target ratio of 10:1. Then, binding proteins at a total of 5 concentrations obtained by a 5-fold gradient dilution (with the maximum final concentration of 6 nM) were added at 50 μL/well, and two duplicate wells were set for each concentration. 30 nM hIgG1 iso (CrownBio, #C0001) was used as an isotype control. The 96-well plate was incubated in an incubator at 37° C. with 5% CO₂. Supernatants after 48 h and 72 h of culture were each collected. The IL-2 concentration in the supernatant after 48 h was determined using an IL-2 ELISA kit (Thermo, #88-7025-88), and the IFN-γ concentration in the supernatant after 72 h was determined using an IFN-γ ELISA kit (Thermo, #88-7316-77). The ELISA assay was performed by referring to the instructions of relevant kit. The data were processed and analyzed by plotting using GraphPad Prism 8 software.

In this example, the anti-4-1BB monoclonal antibody Urelumab was used as a positive control molecule.

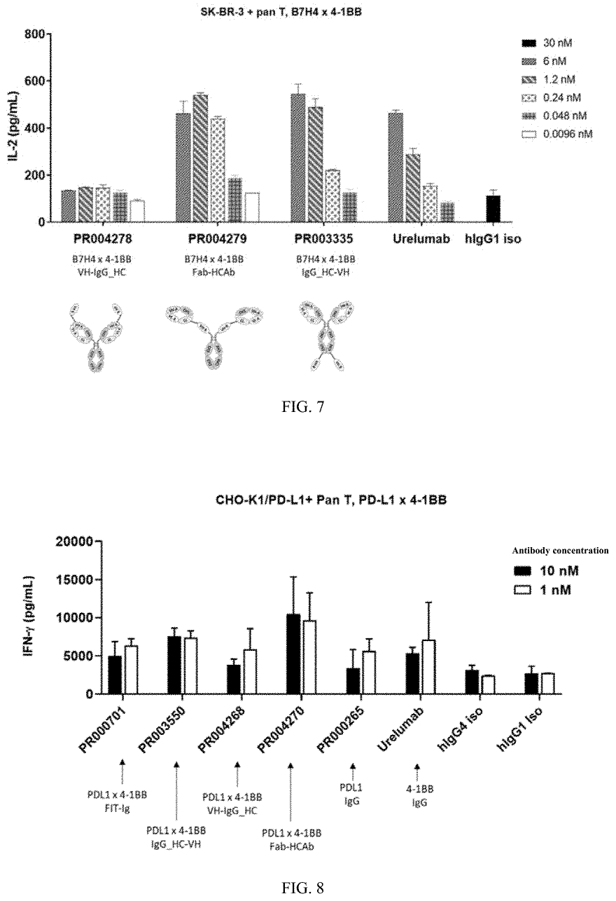

FIG. 7 showed that the binding proteins activates T cells to release IL-2. The molecule with the Fab-HCAb structure (PR004279) and the molecule with the IgG-VH structure (PR003335) had stronger T cell activation ability than Urelumab, and PR004279 was slightly stronger than PR003335. Although the molecule with the VH-IgG structure (PR004278) had strong binding ability to 4-1BB, it was hardly able to activate T cells. This indicates that when the 4-1BB-binding domain VH is located at the N-terminus of the IgG heavy chain, the distance between the target cell-binding domain Fab and the 4-1BB-binding domain VH is not suitable for the formation of an interaction between the target cell and the T cell. Overall, the T cell activation ability is ranked as follows: PR004279>PR003335>Urelumab>PR004278.

### Example 6. B7H4×OX40 Bispecific Binding Proteins

In this example, we constructed B7H4×OX40 bispecific binding proteins with the Fab-HCAb or IgG-VH structure targeting B7H4 and OX40 to specifically activate the immune response in the tumor microenvironment by redirecting OX40 antibodies to tumor cells via the tumor-associated antigen B7H4 through a similar mechanism of action as B7H4×4-1BB.

### Example 6.1 Acquisition of Anti-B7H4 IgG Antibodies and Anti-OX40 HCAb Antibodies

### Example 6.1.1 Acquisition of Fully Human Anti-B7H4 IgG Antibodies

The recombinant fully human anti-B7H4 IgG antibody PR002408 (Table 6) used in this example was derived from Harbour H2L2 mice, and was found as described in Example 5.1.1.

### Example 6.1.2 Acquisition of Fully Human Anti-OX40 HCAb Antibodies

The fully human anti-OX40 HCAb antibody PR002067 (Table 6) used in this example was derived from Harbour HCAb mice, and was found in a similar way to the anti-4-1BB HCAb described in Example 4.1.3. Specifically, the Harbour HCAb mice were subjected to multiple rounds of immunization with a recombinant human OX40-Fc fusion protein (provided by ChemPartner) or a cell strain HEK293/OX40 highly expressing human OX40 (provided by ChemPartner), and subjected to multiple rounds of screening, followed by verification to obtain the fully human anti-OX40 HCAb antibody.

### Example 6.2 Construction of B7H4×OX40 Bispecific Binding Proteins

In one aspect, in this example, an anti-B7H4×OX40 bispecific binding protein with the Fab-HCAb structure described in Example 1.1.1, PR004277, was constructed using Fab of the anti-B7H4 IgG antibody PR002408 and VH of the anti-OX40 HCAb antibody PR002067. The molecular design of PR004277 is shown in Table 4, with the corresponding sequence numbers shown in Table 7. The molecule was prepared and analyzed by the method described in Example 2, with the results summarized in Table 9.

In another aspect, in this example, an anti-B7H4×OX40 bispecific binding protein with the IgG-VH structure described in Example 1.1.3, PR004276, was also constructed using Fab of the anti-B7H4 IgG antibody PR002408 and VH of the anti-OX40 HCAb antibody PR002067. The molecular design of PR004276 is shown in Table 5, with the corresponding sequence numbers shown in Table 7. The molecule was prepared and analyzed by the method described in Example 2, with the results summarized in Table 10.

### Example 6.3 Binding to OX40

In this example, the binding ability of the binding proteins to a CHO-K1 cell strain CHO-K1/hu OX40 (GenScript Biotech, M00561) cells highly expressing human OX40 was determined by flow cytometry FACS. Specifically, the cells were digested and resuspended in an F12K complete medium, and the cell density was adjusted to 1×10⁶ cells/mL. The cells were seeded in a 96-well V-bottom plate (Corning, #3894) at 100 μL/well, followed by the addition of test binding proteins diluted in a 3-fold gradient at a concentration that was 2 times the final concentration, each at 100 μL/well. The cells were incubated at 4° C. for 1 h away from light. Thereafter, the cells in each well were rinsed twice with 100 μL of pre-cooled PBS, and centrifuged at 500 g at 4° C. for 5 min, and then the supernatant was discarded. Then, 100 μL of fluorescent secondary antibody (Alexa Fluor 488-conjugated AffiniPure Goat Anti-Human IgG, Fcγ Fragment Specific, Jackson ImmunoResearch, #109-545-06, diluted in a 1:1000 ratio) was added to each well. The plate was incubated away from light at 4° C. for 30 min. The cells in each well were rinsed twice with 100 μL of pre-cooled PBS, and centrifuged at 500 g at 4° C. for 5 min, and then the supernatant was discarded. Finally, a pre-cooled PBS was added at 200 μL/well to resuspend the cells. Fluorescence signal values were read using a BD FACS CANTOII flow cytometer or an ACEA NovoCyte flow cytometer, and the data were processed and analyzed using FlowJo v10 (FlowJo, LLC) software.

The data were processed and analyzed by plotting using GraphPad Prism 8 software, and binding curves of the binding proteins to target cells, EC₅₀ values and other parameters were obtained through four-parameter nonlinear fitting.

In this example, the anti-OX40 monoclonal antibody Pogalizumab (protein No. PR003475) was used as a positive control molecule.

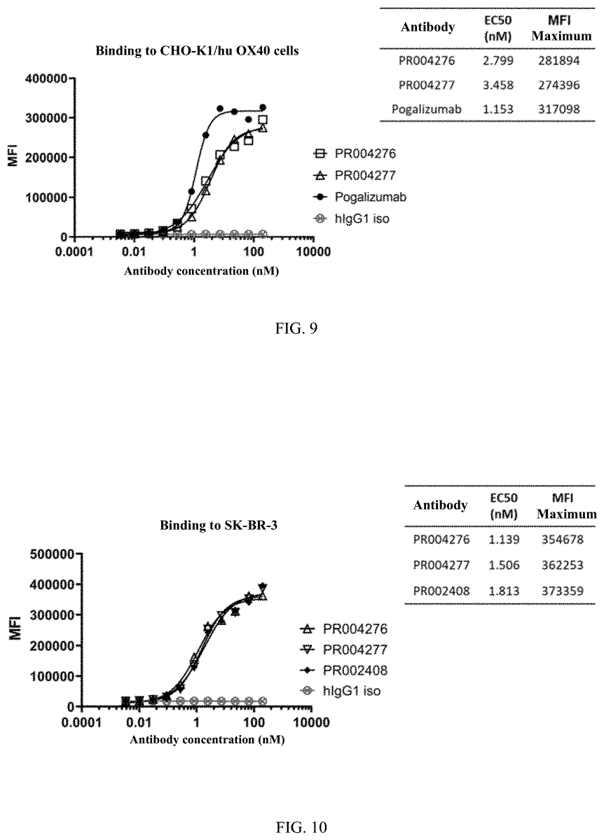

As shown in FIG. 9, the B7H4×OX40 bispecific binding proteins (PR004277 and PR004276) were all able to bind to OX40 with comparable binding ability.

### Example 6.4 Binding to B7H4

In this example, the binding ability of the binding proteins to a tumor cell line SK-BR-3 (ATCC, HTB-30) highly expressing human B7H4 was determined by the method described in Example 5.4. The anti-B7H4 monoclonal antibody PR002408 was used as a positive control molecule, and was also the parent monoclonal antibody of the B7H4 end of B7H4×OX40.

As shown in FIG. 10, the B7H4×OX40 bispecific binding proteins (PR004277 and PR004276) were all able to bind to B7H4 with consistent binding ability with the parent antibody PR002408.

### Example 6.5 T Cell Specific Activation Mediated by Target Cells

This example is intended to investigate the T cell activation activity of the B7H4×OX40 bispecific binding proteins by binding to OX40 in the presence of target cells. The target cells may be CHO-K1/hB7H4 cells (produced in-house by Harbour BioMed) highly expressing human B7H4; and the effector cells may be isolated human PBMC or T cells.

Specifically, a 96-well plate (Corning, #3599) was coated first with 0.3 μg/mL anti-CD3 antibody OKT3 (Thermo, #16-0037-81) at 100 μL/well. Then, the density of human T cells (isolated from human PBMCs with a T cell isolation kit (Miltenyi, #130-096-535)) was adjusted to 2×10⁶ cells/mL, and the density of target cells was adjusted to 3×10⁵ cells/mL. The two cell suspensions were each seeded into a 96-well plate at 50 μL/well. Then, binding proteins at different concentrations were added at 100 μL/well, wherein the binding protein concentration was the final concentration of (20 nM, 2 nM, 0 nM); and two duplicate wells were set for each concentration. hIgG1 iso (CrownBio, #C0001) and a blank well without antibody (no Ab) were used as a control. The 96-well plate was incubated in an incubator at 37° C. with 5% CO₂ for 3 days. Supernatants after 48 h and 72 h of culture were each collected. The IL-2 concentration in the supernatant after 48 h was determined using an IL-2 ELISA kit (Thermo, #88-7025-88), and the IFN-γ concentration in the supernatant after 72 h was determined using an IFN-γ ELISA kit (Thermo, #88-7316-77). The ELISA assay was performed by referring to the instructions of relevant kit. The data were processed and analyzed by plotting using GraphPad Prism 8 software.

In this example, the corresponding parent monoclonal antibodies PR002408 and PR002067 were used as control molecules.

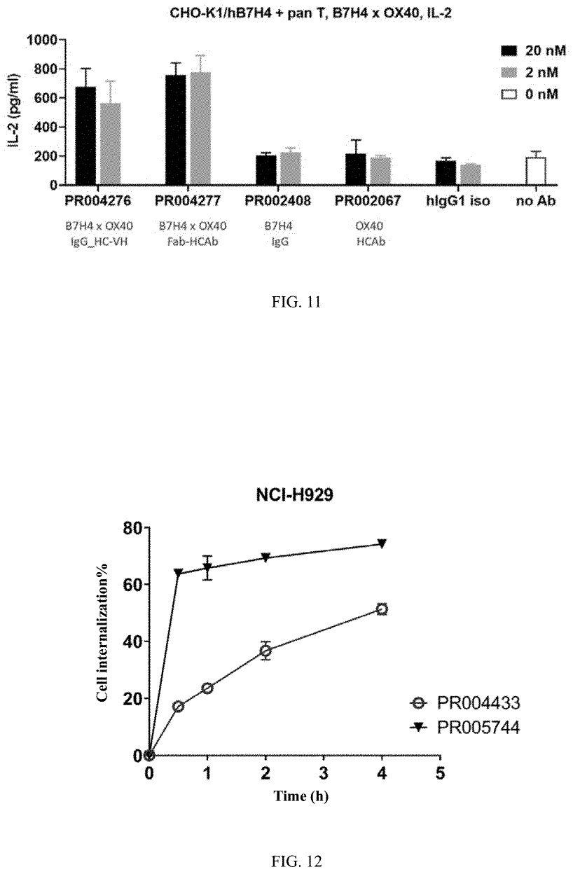

As shown in FIG. 11, in the presence of CHOK1/hB7H4 cells highly expressing B7H4, the anti-OX40 HCAb monoclonal antibody PR002067 and the anti-B7H4 IgG monoclonal antibody PR002408 were both unable to activate T cells; the B7H4×OX40 bispecific binding proteins (PR004277 and PR004276) were both able to activate T cells and promote the production of cytokine IL-2, which indicates that T cell activation by B7H4×OX40 is target cell dependent. Moreover, the molecule with the Fab-HCAb structure (PR004277) had slightly stronger T cell activation ability than the molecule with the IgG-VH structure (PR004276).

### Example 7. BCMA×BCMA Bispecific Binding Proteins

In this example, we constructed multivalent biparatopic bispecific binding proteins with the Fab-HCAb structure targeting BCMA, which can better exploit internalization to achieve killing of target cells.

### Example 7.1 Acquisition of Anti-BCMA Antibodies

### Example 7.1.1 Acquisition of Fully Human Anti-BCMA IgG Antibodies

The recombinant fully human anti-BCMA IgG antibody PR000892 (with the sequences shown in Table 6) used in this example was derived from Harbour H2L2 mice, and its discovery process and sequences were disclosed in patent CN111234020B.

### Example 7.1.2 Acquisition of Fully Human Anti-BCMA HCAb Antibodies

Harbour HCAb mice were subjected to multiple rounds of immunization with a soluble recombinant human BCMA-ECD-Fc fusion protein (ACRO Biosystems, #BC7-H82F0). Screening was performed in a manner similar to that described in Example 4.1.3, and fully human anti-BCMA HCAb antibodies were obtained. Then, the CDR regions of the variable region VH of the HCAb antibody PR001046 were further subjected to two rounds of site-directed mutagenesis to obtain mutants with improved binding affinity for BCMA, e.g., PR001046_R2_4G10 (i.e., PR004433).

The sequences of the recombinant fully human anti-BCMA HCAb antibody PR004433 used in this example are shown in Table 6.

### Example 7.2 Construction of BCMA×BCMA Bispecific Binding Proteins

In this example, an anti-BCMA×BCMA bispecific binding protein with the Fab-HCAb structure described in Example 1.1.1, PR005744, was constructed using Fab of the anti-BCMA IgG antibody PR000892 and VH of the anti-BCMA HCAb antibody PR004433. The molecular design of PR005744 is shown in Table 4, with the corresponding sequence numbers shown in Table 7. The molecule was prepared and analyzed by the method described in Example 2, with the results summarized in Table 9.

Thereafter, the antigen-binding protein PR005744 was determined for its binding ability to BCMA and for its internalization ability on cells NCI-H929 (ATCC, CRL-9068) highly expressing BCMA.

### Example 7.3 Determination of the Affinity of BCMA Binding Proteins for BCMA by the BLI Method

In this example, the binding kinetics between the BCMA binding protein and BCMA were analyzed by the Biolayer Interferometry (BLI) technique using an Octet molecular interaction analyzer (ForteBio, Octet Red96e).

The recombinant human BCMA-ECD-Fc fusion protein (ACRO Biosystems, #BC7-H82F0) was biotinylated using the biotinylation kit (EZ-Link Sulfo-NHS-LC-Biotin, ThermoFisher, A39257) as per the instructions. The sensor used in the experiment was an SA biosensor (ForteBio, #18-5019); the working buffer was 1× kinetics buffer (diluted from 10× kinetics buffer (ForteBio, #18-1105)) for affinity determination and dilution of antigens and binding proteins; the equilibration buffer was 1×PBS buffer (diluted from 10×PBS buffer (BBI Life Sciences, #E607016-0500)).

Two columns of SA sensors (8 sensors in each column; with the sensors in the first column referred to as the reference SA sensor and the sensors in the second column referred to as the test SA sensor) were firstly equilibrated in the equilibration buffer for 10 min. Then, the biotinylated BCMA were captured by test SA sensors with a capture height of 0.2 nm, and the reference SA sensors were immersed in the buffer for 30 s. The two arrays of sensors were combined with the test BCMA-binding protein at concentrations obtained by a two-fold dilution from 10 nM to 2.5 nM and a concentration of 0 nM. The sensors were combined with the test proteins for 180 s and then dissociated for 800 s.

When data analysis was performed using Octet Data Analysis software (Fortebio, version 11.0), the reference signals were subtracted by a double reference mode; the data were fitted by a “1:1 Global fitting” method, and the kinetic parameters of the binding of the antigen to the antigen-binding protein were calculated to obtain kₒₙ (1/Ms) values, k_{dis} (1/s) values and K_{D} (M) values.

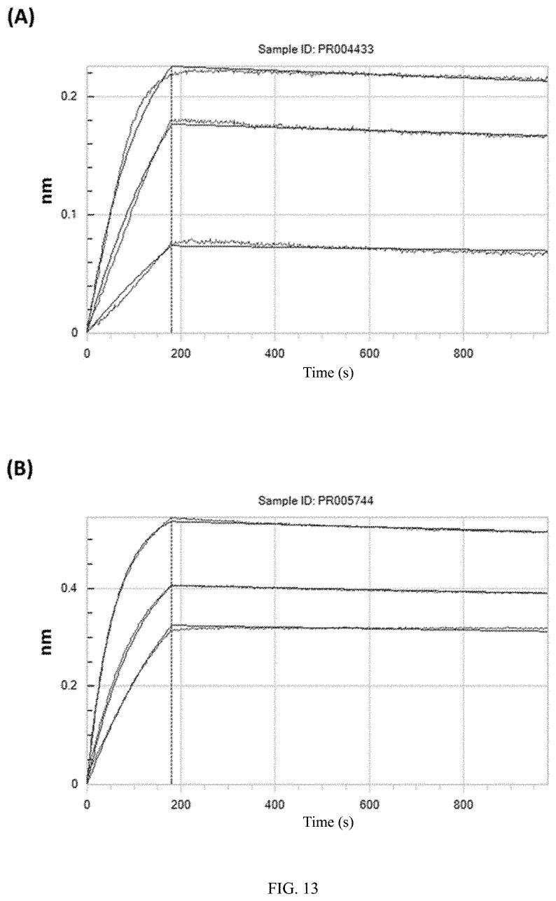

The results are shown in Table 13 and FIG. 13. Tetravalent PR005744 had higher binding affinity (K_{D} value) for BCMA than bivalent PR004433; moreover, PR005744 had higher maximum response signal (Response) than PR004433.

TABLE 13
Binding kinetic parameters of binding proteins to BCMA

| | Binding protein | | | | | |
|---|---|---|---|---|---|---|
| Protein | concentration | KD | kon | kdis | Full | Maximum |
| No. | (nM) | (M) | (1/Ms) | (1/s) | R\hat{ }2 | Response |
| PR004433 | 10-2.5 | 7.92E−11 | 9.04E+05 | 7.16E−05 | 0.9975 | 0.2172 |
| PR005744 | 10-2.5 | 2.79E−11 | 1.75E+06 | 4.87E−05 | 0.9989 | 0.5377 |

### Example 7.4 Internalization of Binding Proteins by the FACS Method

The foregoing examples have demonstrated that tetravalent binding protein (PR005744) has similar or even higher binding ability to BCMA as compared to bivalent binding protein (PR004433). In this example, killing of cells expressing human BCMA mediated by internalization of BCMA-targeted antigen-binding proteins was investigated by the FACS method. Specifically, NCI-H929 (ATCC, CRL-9068) cells were seeded in a 96-well plate (Beyotime, #FT018) at 2×10⁵/well; then, 200 nM test antigen-binding proteins diluted in a FACS buffer was added; then, the plate was incubated at 4° C. for 1 h; thereafter, samples were taken and incubated at 37° C. for various periods of time (e.g., 30 min, 1 h, 2 h, and 4 h); then, the cells were centrifuged and resuspended, and incubated at 4° C. for 30 min after a fluorescent secondary antibody (Jackson ImmunoResearch, #109-545-098) was added. Finally, fluorescence signal values were read using a flow cytometer, and the data were processed and analyzed using FlowJo v10 (FlowJo, LLC) software. The MFI of the fluorescence signal at 0 min (TO) of incubation at 37° C. was taken as a baseline, and the MFIs of samples at different incubation times were subtracted from the baseline of TO and the relative reduction was calculated to reflect the efficiency of internalization of the antigen-binding proteins. The data were processed and analyzed by plotting using GraphPad Prism 8 software.

As shown in FIG. 12, PR005744 had significantly superior internalization effect in NCI-H929 cells to PR004433; it allowed more than 60% of BCMA to be internalized within 30 min.

### Example 8. HER2×CTLA4 Bispecific Binding Proteins

In this example, we constructed multiple HER2×CTLA4 bispecific binding proteins with the Fab-HCAb structure targeting HER2 and CTLA4. HER2×CTLA4 can be enriched in tumor tissue highly expressing HER2 and can specifically relieve CTLA4 inhibition signals in a tumor microenvironment to activate T cells, so that toxic and side effects caused by non-specific activation by CTLA4 monoclonal antibodies in a peripheral system are reduced. In this example, multiple molecules with the Fab-HCAb structure containing different linker peptide were constructed to investigate the effect of linker peptides on the Fab-HCAb molecular structure.

### Example 8.1 Acquisition of Anti-HER2 IgG Antibodies and Anti-CTLA4 HCAb Antibodies

### Example 8.1.1 Acquisition of Anti-HER2 IgG Antibodies

For the anti-HER2 IgG antibody trastuzumab (protein No. PR000210) used in this example, the corresponding amino acid sequence was derived from the IMGT database, and the sequences were shown in Table 6.

### Example 8.1.2 Acquisition of Fully Human Anti-CTLA4 HCAb Antibodies

Harbour HCAb mice were subjected to multiple rounds of immunization with a soluble recombinant human CTLA4 protein (ACRO Biosystems, #CT4-H5229). Screening was performed in a manner similar to that described in Example 4.1.3, and fully human anti-CTLA4 HCAb antibodies were obtained.

The sequences of the recombinant fully human anti-CTLA4 HCAb antibody PR000184 used in this example are shown in Table 6.

### Example 8.2 Construction of HER2×CTLA4 Bispecific Binding Proteins

In this example, anti-HER2×CTLA4 bispecific binding proteins with the Fab-HCAb structure described in Example 1.1.1, PR000305, PR000653, PR000654, PR000655 and PR000706, were constructed using Fab of the anti-HER2 IgG antibody PR000210 (trastuzumab analog) and VH of the anti-CTLA4 HCAb antibody PR000184. The molecular designs are shown in Table 4, with the corresponding sequence numbers shown in Table 7. The molecules were prepared and analyzed by the method described in Example 2, with the results summarized in Table 9. Those bispecific binding protein molecules had similar structures with the identical antigen-binding domains Fab and VH, with minor differences in the sequences of the different first (between Fab and VH) and second (between VH and CH2) linker peptides.

In this example, those molecules were used to investigate the effect of different linker peptides on the Fab-HCAb molecular structure.

### Example 8.3 Binding to HER2

In this example, the binding ability of the binding proteins to a tumor cell line SK-BR-3 (ATCC, HTB-30) highly expressing human HER2 was determined by flow cytometry FACS. Specifically, the SK-BR-3 cells were digested and resuspended in a complete medium, and the cell density was adjusted to 1×10⁶ cells/mL. Then, the cells were seeded in a 96-well V-bottom plate (Corning, #3894) at 100 μL/well and centrifuged at 4° C. for 5 min, and the supernatant was discarded. Then, the binding proteins at a total of 8 concentrations obtained by a 5-fold gradient dilution with the highest final concentration of 100 nM were added at 100 μL/well. The mixture was mixed well. hIgG1 iso (CrownBio, #C0001) was used as an isotype control. The cells were incubated at 4° C. for 1 h away from light. Thereafter, the mixture was centrifuged at 4° C. for 5 min, and the supernatant was discarded. Then, the cells in each well were rinsed twice with 200 μL of pre-cooled FACS buffer (PBS buffer containing 0.5% BSA), and centrifuged at 500 g at 4° C. for 5 min, and then the supernatant was discarded. Thereafter, a fluorescent secondary antibody (Goat human IgG (H+L) Alexa Fluor 488 conjugation, Thermo, #A11013, diluted in a 1:1000 ratio) was added at 100 μL/well, and the plate was incubated at 4° C. for 1 h away from light. Then, the cells in each well were then rinsed twice with 200 μL of pre-cooled FACS buffer and centrifuged at 500 g at 4° C. for 5 min, and then the supernatant was discarded. Finally, a pre-cooled FACS buffer was added at 200 μL/well to resuspend the cells. Fluorescence signal values were read using a BD FACS CANTOII flow cytometer, and the data were processed and analyzed using FlowJo v10 (FlowJo, LLC) software.

The data were processed and analyzed by plotting using GraphPad Prism 8 software, and binding curves of the antibodies to target cells, EC₅₀ values and other parameters were obtained through four-parameter nonlinear fitting.

In this example, the anti-HER2 monoclonal antibody PR000210 (trastuzumab analog) was used as a positive control molecule, and was also the parent monoclonal antibody of the HER2 end of HER2×CTLA4.

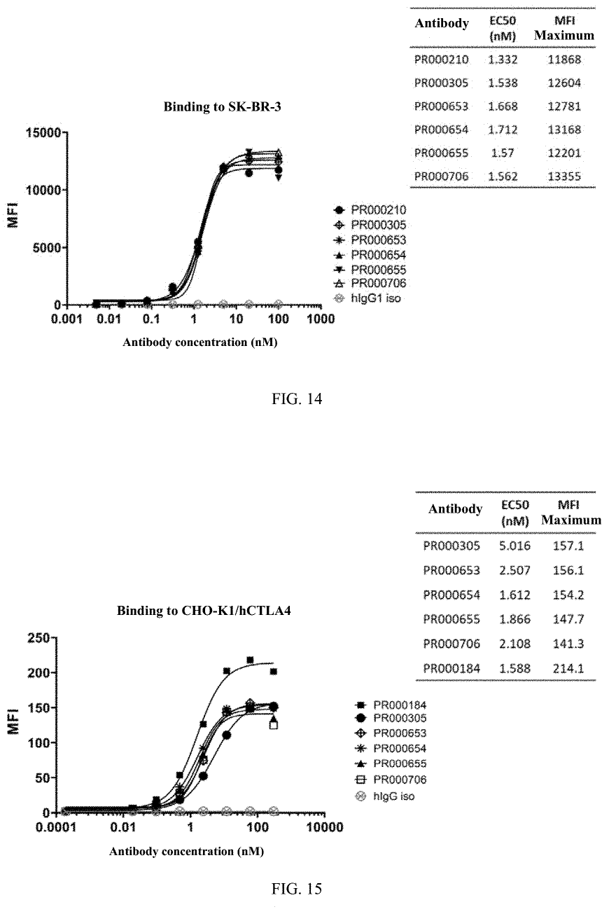

As shown in FIG. 14, the bispecific binding proteins with the Fab-HCAb structure (PR000305, PR000653, PR000654, PR000655 and PR000706) had comparable binding ability to HER2 to the parental monoclonal antibody PR000210, which was reflected in nearly identical EC₅₀ values and maximum MFI values. This indicates that the Fab end of the Fab-HCAb structure can well retain its corresponding target-binding ability.

### Example 8.4 Binding to CTLA4

In this example, the binding ability of the binding proteins to a CHO-K1 cell strain CHO-K1/hCTLA4 cells (ChemPartner) and other cells highly expressing human CTLA4 was determined by flow cytometry FACS. Specifically, CHO-K1/hCTLA4 cells were digested and resuspended in an F12K medium, with the cell density adjusted to 2×10⁶ cells/mL. Thereafter, the CHO-K1/hCTLA4 cells were seeded in a 96-well V-bottom plate (Corning, #3894) at 100 μL/well and centrifuged at 4° C. for 5 min, and the supernatant was discarded. Then, the binding proteins at a total of 8 concentrations obtained by a 5-fold gradient dilution with the highest final concentration of 300 nM were added at 100 μL/well. The mixture was mixed well. hIgG1 iso (CrownBio, #C0001) was used as an isotype control. The cells were incubated at 4° C. for 1 h away from light. Then, the cells in each well were rinsed twice with 100 μL of pre-cooled FACS buffer (PBS buffer containing 0.5% BSA) and centrifuged at 500 g at 4° C. for 5 min, and the supernatant was discarded. Thereafter, a fluorescent secondary antibody (Goat human IgG (H+L) Alexa Fluor 488 conjugation, Thermo, #A11013, diluted in a 1:1000 ratio) was added at 100 μL/well, and the plate was incubated at 4° C. for 1 h away from light. Then, the cells in each well were then rinsed twice with 200 μL of pre-cooled FACS buffer and centrifuged at 500 g at 4° C. for 5 min, and then the supernatant was discarded. Finally, a pre-cooled FACS buffer was added at 200 μL/well to resuspend the cells. Fluorescence signal values were read using a BD FACS CANTOII flow cytometer, and the data were processed and analyzed using FlowJo v10 (FlowJo, LLC) software.

The data were processed and analyzed by plotting using GraphPad Prism 8 software, and binding curves of the antibodies to target cells, EC₅₀ values and other parameters were obtained through four-parameter nonlinear fitting.

In this example, the anti-CTLA4 HCAb monoclonal antibody PR000184 was used as a positive control molecule, and was also the parent monoclonal antibody of the CTLA4 end of HER2×CTLA4.

As shown in FIG. 15, the bispecific binding proteins with the Fab-HCAb structure (PR000305, PR000653, PR000654, PR000655 and PR000706) were all able to bind to CTLA4. Those molecules had similar structures with the identical VH sequence at the end of CTLA4, with minor differences in the different first linker peptides and hinge regions linking the Fc. Therefore, those molecules had very similar binding ability to CTLA4. This indicates that the length or sequence of different linker peptides has little effect on the binding domain VH in the Fab-HCAb structure.

In another aspect, those molecules had EC₅₀ values for binding to CTLA4 that were similar to or 1.5-3 times poorer than the parent monoclonal antibody PR000184, but had lower maximum binding signals (maximum MFIs) on FACS than the parent monoclonal antibody PR000184. This may suggest that in some application scenarios for the Fab-HCAb structure, the Fab domain may have a “masking” effect on the VH domain of the HCAb, so that the Fab-HCAb molecule may preferentially bind to the target recognized by the Fab domain before causing binding of the VH domain. The binding in sequence and the difference in the binding force of different targets can be suitable for the requirements of some special application scenarios. For example, the recommended initial dose of the anti-HER2 monoclonal antibody trastuzumab is 4 mg/kg for the treatment of breast cancer and 8 mg/kg for the treatment of gastric cancer; while the recommended initial dose of the anti-CTLA4 monoclonal antibody ipilimumab was 3 mg/kg for the treatment of melanoma and lower in combination therapy. For the HER2×CTLA4 with the Fab-HCAb structure, the activity of the HER2 end is almost comparable to that of their parent monoclonal antibody, but the activity of the CTLA4 end is relatively weakened. Therefore, this structure can be used to achieve the clinical requirements for moderate or low doses of CTLA4 inhibitors. In addition, HER2×CTLA4 can preferentially bind to HER2 and enriched in tumor tissues highly expressing HER2, so that toxic and side effects caused by non-specific activation of T cells by CTLA4 antibodies in a peripheral system are reduced.

### Example 9. Pharmacokinetic Study of Molecules with Fab-HCAb Structure

In this example, the pharmacokinetic properties of a bispecific binding protein molecule with the Fab-HCAb structure, PR004270 (with sequences shown in Table 7), in mice were investigated.

Administration and blood collection: for each test antibody molecule, 6 female BALB/C or C57BL/6 mice weighing 18-22 g were selected and administered with the test antibody molecule intravenously at a dose of 5 mg/kg. The whole blood of 3 mice in one group was collected before the administration and 15 min, 24 h (1 day), 4 days and 10 days after the administration, and the whole blood of 3 mice in the other group was collected before the administration and 5 h, 2 days, 7 days and 14 days after the administration. The whole blood was left to stand for 30 min for coagulation, and then centrifuged, and the isolated serum sample was cryopreserved at −80° C. until it was taken for analysis.

Analysis method: the drug concentration in the serum of mice was quantitatively determined by two ELISA methods. ELISA method I, namely the Fc end detection method, was performed by capturing human Fc-containing antibodies in the serum of mice using a goat anti-human Fc polyclonal antibody coating a 96-well plate, and then adding an HRP-labeled goat anti-human Fc secondary antibody. ELISA method II, namely the functional domain detection method, was performed by capturing the antibodies specifically recognizing the antigens in the serum of mice using a PD-L1 protein coating a 96-well plate, and then adding an HRP-labeled goat anti-human Fc secondary antibody. Finally, pharmacokinetic parameters were analyzed using a non-compartmental analysis (NCA) model of Phoenix WinNonlin software (version 8.2).

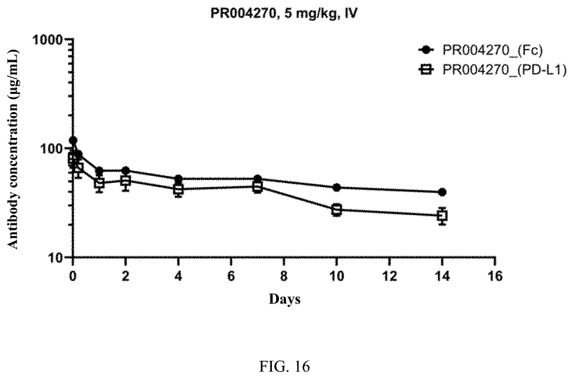

As shown in FIG. 16 and Table 14, the molecule with the Fab-HCAb structure (PR004270) had a similar serum half-life t_{1/2} value to a conventional IgG antibody, and the t_{1/2} value was more than 10 days as measured by the PD-L1 end detection method.

TABLE 14
Pharmacokinetic parameters in mice

| Bispecific antibody molecule | PR004270 | |
|---|---|---|
| Animals (number) | BALB/c mice (n = 6) | |
| Antibody dosage | 5 mg/kg, I.V. | |
| PK parameters | Fc end detection | PD-L1 end detection |
| T_{1/2} (hour) | 465.6 | 256.5 |
| V_{d} (mL/kg) | 75.7 | 83.9 |
| AUC(μg*hour/mL) | 17,536 | 13,126 |
| Cl (mL/hour/kg) | 0.11 | 0.23 |
| C₀ (μg/mL) | 119.7 | 81.7 |

This example demonstrates that molecules with the Fab-HCAb structure have excellent pharmacokinetic properties.

Although specific embodiments of the present invention have been described above, it will be appreciated by those skilled in the art that these embodiments are merely illustrative and that many changes or modifications can be made to these embodiments without departing from the principles and spirit of the present invention. The scope of protection of the present invention is therefore defined by the appended claims.

What is claimed is:

1. A binding protein comprising at least two protein functional regions, wherein the binding protein comprises a protein functional region A and a protein functional region B; the protein functional region A and the protein functional region B target different antigens or different epitopes on the same antigen;
wherein the protein functional region A is of a Fab structure; the protein functional region B is of a VH structure, and the binding protein further comprises an Fc homodimer;
the number of the protein functional region A is two, and the number of the protein functional region B is two; the binding protein is of a left-right symmetric structure;
the binding protein comprises a protein functional region A, a protein functional region B and an Fc homodimer sequentially from the N-terminus to the C-terminus, wherein the protein functional region A is linked to the protein functional region B via a first linker peptide (L1), and the protein functional region B is linked to the Fc via a second linker peptide (L2).

2. The binding protein according to claim 1, wherein the binding protein has four polypeptide chains, including two identical short chains and two identical long chains, wherein (1) the short chain comprises VH_A-CH1 sequentially from the N-terminus to the C-terminus, and the long chain comprises VL_A-CL-L1-VH_B-L2-CH2-CH3 sequentially from the N-terminus to the C-terminus; or (2) the short chain comprises VL_A-CL sequentially from the N-terminus to the C-terminus, and the long chain comprises VH_A-CH1-L1-VH_B-L2-CH2-CH3 sequentially from the N-terminus to the C-terminus;
wherein the VL_A and the VH_A are VL and VH of the protein functional region A, respectively, and the VH_B is VH of the protein functional region B; the CL is a domain of a light chain constant region; the CH1, CH2 and CH3 are a first domain, a second domain and a third domain of a heavy chain constant region, respectively; and the L1 or L2 is a linker peptide;
preferably, the L1 and L2 are independently, e.g., “-” or GS or have amino acid sequences as shown in SEQ ID NOs: 161-182, respectively.

3. The binding protein according to claim 1, wherein the antigen is selected from one or more of PD-L1, HER2, B7H4, CTLA4, OX40, 4-1BB and BCMA;
preferably,
the protein functional region A is Fab derived from a PD-L1 antibody or an antigen-binding fragment thereof, an HER2 antibody or an antigen-binding fragment thereof, a B7H4 antibody or an antigen-binding fragment thereof, or a BCMA antibody or an antigen-binding fragment thereof, and, the protein functional region B is VH derived from a CTLA4 antibody or an antigen-binding fragment thereof, a 4-1BB antibody or an antigen-binding fragment thereof, an OX40 antibody or an antigen-binding fragment thereof, or a BCMA antibody or an antigen-binding fragment thereof.

4. The binding protein according to claim 3, wherein
the PD-L1 antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 75, 85 and 97, respectively; and the VH comprises HCDR1, HCDR2 and HCDR3 with amino acid sequences as set forth in SEQ ID NOs: 13, 32 and 54, respectively;
the B7H4 antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 78, 83 and 100, respectively; and the VH comprises HCDR1, HCDR2 and HCDR3 with amino acid sequences as set forth in SEQ ID NOs: 15, 37 and 59, respectively;
the 4-1BB antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 73, 83 and 95, respectively; and the VH comprises HCDR1, HCDR2 and HCDR3 with amino acid sequences as set forth in SEQ ID NOs: 11, 30 and 52, respectively; or, the 4-1BB antibody or the antigen-binding fragment thereof comprises a heavy chain variable region (VH), wherein the VH comprises HCDR1, HCDR2 and HCDR3 with amino acid sequences as set forth in SEQ ID NOs: 14, 35 and 57, respectively;
the OX40 antibody or the antigen-binding fragment thereof comprises a heavy chain variable region (VH), wherein the VH comprises HCDR1, HCDR2 and HCDR3 with amino acid sequences as set forth in SEQ ID NOs: 13, 36 and 58, respectively;
the BCMA antibody or the antigen-binding fragment thereof comprises a heavy chain variable region (VH), wherein the VH comprises HCDR1, HCDR2 and HCDR3 with amino acid sequences as set forth in SEQ ID NOs: 17, 39 and 61, respectively; or, the BCMA antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 77, 87 and 99, respectively; and the VH comprises HCDR1, HCDR2 and HCDR3 with amino acid sequences as set forth in SEQ ID NOs: 13, 34 and 56, respectively;
the CTLA4 antibody or the antigen-binding fragment thereof comprises a heavy chain variable region (VH), wherein the VH comprises HCDR1, HCDR2 and HCDR3 with amino acid sequences as set forth in SEQ ID NOs: 10, 29 and 51, respectively; or
the HER2 antibody or the antigen-binding fragment thereof comprises a light chain variable region (VL) and a heavy chain variable region (VH), wherein the VL comprises LCDR1, LCDR2 and LCDR3 with amino acid sequences as set forth in SEQ ID NOs: 74, 84 and 96, respectively; and the VH comprises HCDR1, HCDR2 and HCDR3 with amino acid sequences as set forth in SEQ ID NOs: 12, 31 and 53, respectively.

5. The binding protein according to claim 3, wherein
the PD-L1 antibody or the antigen-binding fragment thereof comprises a light chain variable region (VL) and a heavy chain variable region (VH), wherein the VL comprises an amino acid sequence as set forth in SEQ ID NO: 118, and the VH comprises an amino acid sequence as set forth in SEQ ID NO: 108;
the B7H4 antibody or the antigen-binding fragment thereof comprises a light chain variable region (VL) and a heavy chain variable region (VH), wherein the VL comprises an amino acid sequence as set forth in SEQ ID NO: 121, and the VH comprises an amino acid sequence as set forth in SEQ ID NO: 113;
the 4-1BB antibody or the antigen-binding fragment thereof comprises a light chain variable region (VL) and a heavy chain variable region (VH), wherein the VL comprises an amino acid sequence as set forth in SEQ ID NO: 116, and the VH comprises an amino acid sequence as set forth in SEQ ID NO: 106; or, the 4-1BB antibody or the antigen-binding fragment thereof comprises a heavy chain variable region (VH), wherein the VH comprises an amino acid sequence as set forth in SEQ ID NO: 111;
the OX40 antibody or the antigen-binding fragment thereof comprises a heavy chain variable region (VH), wherein the VH comprises an amino acid sequence as set forth in SEQ ID NO: 112;
the BCMA antibody or the antigen-binding fragment thereof comprises a heavy chain variable region (VH), wherein the VH comprises an amino acid sequence as set forth in SEQ ID NO: 115; or, the BCMA antibody or the antigen-binding fragment thereof comprises a light chain variable region (VL) and a heavy chain variable region (VH), wherein the VL comprises an amino acid sequence as set forth in SEQ ID NO: 120, and the VH comprises an amino acid sequence as set forth in SEQ ID NO: 110;
the CTLA4 antibody or the antigen-binding fragment thereof comprises a heavy chain variable region (VH), wherein the VH comprises an amino acid sequence as set forth in SEQ ID NO: 105; and
the HER2 antibody or the antigen-binding fragment thereof comprises a light chain variable region (VL) and a heavy chain variable region (VH), wherein the VL comprises an amino acid sequence as set forth in SEQ ID NO: 117, and the VH comprises an amino acid sequence as set forth in SEQ ID NO: 107.

6. The binding protein according to claim 1, wherein the binding protein comprises a protein functional region A and a protein functional region B, wherein:
the protein functional region A comprises a light chain variable region (VL) and a heavy chain variable region (VH), wherein the VL comprises LCDR1, LCDR2 and LCDR3 with amino acid sequences as set forth in SEQ ID NOs: 75, 85 and 97, respectively; and the VH comprises HCDR1, HCDR2 and HCDR3 with amino acid sequences as set forth in SEQ ID NOs: 13, 32 and 54, respectively; and, the protein functional region B comprises a heavy chain variable region (VH), wherein the VH comprises HCDR1, HCDR2 and HCDR3 with amino acid sequences as set forth in SEQ ID NOs: 14, 35 and 57, respectively; or,
the protein functional region A comprises a light chain variable region (VL) and a heavy chain variable region (VH), wherein the VL comprises LCDR1, LCDR2 and LCDR3 with amino acid sequences as set forth in SEQ ID NOs: 78, 83 and 100, respectively; and the VH comprises HCDR1, HCDR2 and HCDR3 with amino acid sequences as set forth in SEQ ID NOs: 15, 37 and 59, respectively; and, the protein functional region B comprises a heavy chain variable region (VH), wherein the VH comprises HCDR1, HCDR2 and HCDR3 with amino acid sequences as set forth in SEQ ID NOs: 14, 35 and 57, respectively; or,
the protein functional region A comprises a light chain variable region (VL) and a heavy chain variable region (VH), wherein the VL comprises LCDR1, LCDR2 and LCDR3 with amino acid sequences as set forth in SEQ ID NOs: 78, 83 and 100, respectively; and the VH comprises HCDR1, HCDR2 and HCDR3 with amino acid sequences as set forth in SEQ ID NOs: 15, 37 and 59, respectively; and, the protein functional region B comprises a heavy chain variable region (VH), wherein the VH comprises HCDR1, HCDR2 and HCDR3 with amino acid sequences as set forth in SEQ ID NOs: 13, 36 and 58, respectively; or,
the protein functional region A comprises a light chain variable region (VL) and a heavy chain variable region (VH), wherein the VL comprises LCDR1, LCDR2 and LCDR3 with amino acid sequences as set forth in SEQ ID NOs: 77, 87 and 99, respectively; and the VH comprises HCDR1, HCDR2 and HCDR3 with amino acid sequences as set forth in SEQ ID NOs: 13, 34 and 56, respectively; and, the protein functional region B comprises a heavy chain variable region (VH), wherein the VH comprises HCDR1, HCDR2 and HCDR3 with amino acid sequences as set forth in SEQ ID NOs: 17, 39 and 61, respectively; or,
the protein functional region A comprises a light chain variable region (VL) and a heavy chain variable region (VH), wherein the VL comprises LCDR1, LCDR2 and LCDR3 with amino acid sequences as set forth in SEQ ID NOs: 74, 84 and 96, respectively; and the VH comprises HCDR1, HCDR2 and HCDR3 with amino acid sequences as set forth in SEQ ID NOs: 12, 31 and 53, respectively; and, the protein functional region B comprises a heavy chain variable region (VH), wherein the VH comprises HCDR1, HCDR2 and HCDR3 with amino acid sequences as set forth in SEQ ID NOs: 10, 29 and 51, respectively.

7. The binding protein according to claim 1, wherein the binding protein comprises a protein functional region A and a protein functional region B, wherein:
the protein functional region A comprises a light chain variable region with an amino acid sequence as set forth in SEQ ID NO: 118 and a heavy chain variable region with an amino acid sequence as set forth in SEQ ID NO: 108; and the protein functional region B comprises a heavy chain variable region with an amino acid sequence as set forth in SEQ ID NO: 111; or,
the protein functional region A comprises a light chain variable region with an amino acid sequence as set forth in SEQ ID NO: 121 and a heavy chain variable region with an amino acid sequence as set forth in SEQ ID NO: 113; and the protein functional region B comprises a heavy chain variable region with an amino acid sequence as set forth in SEQ ID NO: 111; or,
the protein functional region A comprises a light chain variable region with an amino acid sequence as set forth in SEQ ID NO: 121 and a heavy chain variable region with an amino acid sequence as set forth in SEQ ID NO: 113; and the protein functional region B comprises a heavy chain variable region with an amino acid sequence as set forth in SEQ ID NO: 112; or,
the protein functional region A comprises a light chain variable region with an amino acid sequence as set forth in SEQ ID NO: 120 and a heavy chain variable region with an amino acid sequence as set forth in SEQ ID NO: 110; and the protein functional region B comprises a heavy chain variable region with an amino acid sequence as set forth in SEQ ID NO: 115; or,
the protein functional region A comprises a light chain variable region with an amino acid sequence as set forth in SEQ ID NO: 117 and a heavy chain variable region with an amino acid sequence as set forth in SEQ ID NO: 107; and the protein functional region B comprises a heavy chain variable region with an amino acid sequence as set forth in SEQ ID NO: 105.

8. The binding protein according to claim 1, comprising two different polypeptide chains: a first polypeptide chain and a second polypeptide chain, wherein
the first polypeptide chain comprises an amino acid sequence as set forth in SEQ ID NO: 147; and the second polypeptide chain comprises an amino acid sequence as set forth in SEQ ID NO: 153; or,
the first polypeptide chain comprises an amino acid sequence as set forth in SEQ ID NO: 136; and the second polypeptide chain comprises an amino acid sequence as set forth in SEQ ID NO: 183; or,
the first polypeptide chain comprises an amino acid sequence as set forth in SEQ ID NO: 147; and the second polypeptide chain comprises an amino acid sequence as set forth in SEQ ID NO: 184; or,
the first polypeptide chain comprises an amino acid sequence as set forth in SEQ ID NO: 155; and the second polypeptide chain comprises an amino acid sequence as set forth in SEQ ID NO: 158; or,
the first polypeptide chain comprises an amino acid sequence as set forth in SEQ ID NO: 155; and the second polypeptide chain comprises an amino acid sequence as set forth in SEQ ID NO: 156; or,
the first polypeptide chain comprises an amino acid sequence as set forth in SEQ ID NO: 159; and the second polypeptide chain comprises an amino acid sequence as set forth in SEQ ID NO: 160; or,
the first polypeptide chain comprises an amino acid sequence as set forth in SEQ ID NO: 141; and the second polypeptide chain comprises an amino acid sequence as set forth in SEQ ID NO: 142; or,
the first polypeptide chain comprises an amino acid sequence as set forth in SEQ ID NO: 141; and the second polypeptide chain comprises an amino acid sequence as set forth in SEQ ID NO: 143; or,
the first polypeptide chain comprises an amino acid sequence as set forth in SEQ ID NO: 141; and the second polypeptide chain comprises an amino acid sequence as set forth in SEQ ID NO: 144; or,
the first polypeptide chain comprises an amino acid sequence as set forth in SEQ ID NO: 141; and the second polypeptide chain comprises an amino acid sequence as set forth in SEQ ID NO: 145; or,
the first polypeptide chain comprises an amino acid sequence as set forth in SEQ ID NO: 141; and the second polypeptide chain comprises an amino acid sequence as set forth in SEQ ID NO: 149.

9. The binding protein according to claim 1, wherein in the binding protein, the light chain constant region (CL) of the Fab structure is a human light chain constant region, preferably a human light chain constant region Cκ or Cλ;
and, in the binding protein, the heavy chain constant region (CH) is a human heavy chain constant region, preferably a human IgG1, IgG2, IgG3 or IgG4 heavy chain constant region or a mutation thereof, wherein the mutation is preferably one or more mutations selected from C220S, N297A, L234A, L235A, G237A and P329G, and sites of the mutations are numbered according to the EU numbering scheme.

10. An isolated nucleic acid encoding the binding protein according to claim 1.

11. A recombinant expression vector comprising the isolated nucleic acid according to claim 10; preferably, the expression vector comprises a eukaryotic cell expression vector or a prokaryotic cell expression vector.

12. A transformant comprising the isolated nucleic acid according to claim 10;
preferably, the transformant has a host being a prokaryotic cell or a eukaryotic cell, wherein the prokaryotic cell is preferably an E. coli cell such as TG1 and BL21, and the eukaryotic cell is preferably an HEK293 cell or a CHO cell.

13. A method for preparing a binding protein comprising culturing the transformant according to claim 12, and obtaining the binding protein from a culture.

14. A pharmaceutical composition comprising the binding protein according to claim 1, and a pharmaceutically acceptable carrier;
preferably, the pharmaceutical composition further comprises an additional anti-tumor antibody as an active ingredient.

15. A kit comprising the binding protein according to claim 1;
preferably, the kit further comprises (i) a device for administering the binding protein; or (ii) instructions for use.

16. A combination of kits comprising a kit I and a kit II, wherein the kit I comprises the binding protein according to claim 1, and the kit II comprises an additional antibody or pharmaceutical composition.

17. An administration device comprising the binding protein according to claim 1;
preferably, the administration device further comprises a component, such as a syringe, an infusion device or an implantable administration device, for containing or administering the binding protein to a subject.

18. (canceled)

19. A method for detecting a specific antigen in vitro or in vivo comprising using the binding protein according to claim 1 for detection.

20. (canceled)

21. A method for diagnosing, preventing or treating cancer or other diseases comprising the step of administering to a patient in need thereof the binding protein according to claim 1;
preferably, the cancer is selected from one or more of breast cancer, ovarian cancer, endometrial cancer, renal cancer, melanoma, lung cancer, gastric cancer, liver cancer, esophageal cancer, cervical cancer, head and neck neoplasm, cholangiocarcinoma, gallbladder cancer, bladder cancer, sarcoma, colorectal cancer, lymphoma and multiple myeloma.

22. The binding protein according to claim 3, wherein the protein functional region A is Fab derived from a HER2 antibody or an antigen-binding fragment thereof, and the protein functional region B is VH derived from a CTLA4 antibody or an antigen-binding fragment thereof; or, the protein functional region A is Fab derived from a PD-L1 antibody or an antigen-binding fragment thereof, and the protein functional region B is VH derived from a 4-1BB antibody or an antigen-binding fragment thereof; or, the protein functional region A is Fab derived from a B7H4 antibody or an antigen-binding fragment thereof, and the protein functional region B is VH derived from a 4-1BB antibody or an antigen-binding fragment thereof; or, the protein functional region A is Fab derived from a B7H4 antibody or an antigen-binding fragment thereof, and the protein functional region B is VH derived from an OX40 antibody or an antigen-binding fragment thereof; or, the protein functional region A is Fab derived from a BCMA antibody or an antigen-binding fragment thereof, and the protein functional region B is VH derived from a BCMA antibody or an antigen-binding fragment thereof.
